(19) United States
(12) Patent Application Publication
(10) Pub. No.: US 2023/0175010 A1
(43) Pub. Date: Jun. 8, 2023

(54) COMPOSITIONS AND METHODS FOR BASE SPECIFIC MITOCHONDRIAL GENE EDITING

(71) Applicant: City of Hope, Duarte, CA (US)

(72) Inventors: Shasha Li, Arcadia, CA (US); John Cory Burnett, Chatsworth, CA (US)

(21) Appl. No.: 17/998,469

(22) PCT Filed: May 12, 2021
(86) PCT No.: PCT/US2021/032035

§ 371 Date: Nov. 10, 2022

Related U.S. Application Data
(60) Provisional application No. 63/023,841, filed on May 12, 2020.

(51) Int. Cl. C12N 15/85; C12N 9/22

(52) U.S. Cl. CPC C12N 15/85; C12N 9/22; C12Y 115/01001; C12Y 305/04004; C12Y 305/04005; C07K 2319/07; C07K 2319/09

(57) ABSTRACT

Provided herein are compositions that include proteins that include a mitochondrial localization amino acid sequence attached to a base editor fusion protein including a nucleotide base-modifying enzyme and an RNA-guided DNA endonuclease enzyme with modified endonuclease activity. Provided herein are nucleic acids encoding the proteins and vectors including the nucleic acids. Provided herein are pharmaceutical compositions including the compositions, proteins, nucleic acid and vectors. Provided herein are related methods for modifying mitochondrial DNA and treating mitochondrial disorders.

### CROSS-REFERENCES TO RELATED APPLICATIONS

This application claims priority benefit to U.S. provisional 63/023,841 filed May 12, 2020, which is incorporated herein in its entirety.

### REFERENCE TO A “SEQUENCE LISTING,” A TABLE, OR A COMPUTER PROGRAM LISTING APPENDIX SUBMITTED ON A COMPACT DISK

This application incorporates by reference a Computer Readable Form (CRF) of a Sequence Listing in ASCII text format submitted with this application, entitled 048440-767001WO_SequenceListing_ST25.TXT, was created on May 10, 2021, and is 73,728 bytes in size.

### BACKGROUND OF THE INVENTION

Mitochondria are essential organelles that generate the bulk of cellular energy in the form of ATP from the oxidation of carbohydrates and fats. To carry out this central role in bioenergetics, mitochondria require their own genome, a 16.6 kilobase (kb) circular double-stranded molecule that encodes 37 genes. Each human cell carries hundreds to thousands of copies of mitochondria DNA (mtDNA). In mitochondrial encephalopmyopathies, cells typically contain a mixture of both pathogenic and normal mtDNA molecules, a state termed heteroplasmy. There are over 600 known mtDNA mutations associated with such mtDNA diseases, which have diverse clinical features, including maternal inheritance, defects in the central and peripheral nervous systems, muscle defects, and exercise intolerance. Due to the inability to transform mitochondrial DNA, there are no approved clinical therapies for the treatment of mitochondrial diseases. Provided herein are solutions to these and other problems in the art.

### BRIEF SUMMARY OF THE INVENTION

In a first aspect is provided a composition including a delivery vehicle and a protein, said protein including a mitochondrial localization amino acid sequence covalently attached to a base editor fusion protein including a nucleotide base-modifying enzyme and an RNA-guided DNA endonuclease enzyme with modified endonuclease activity, and wherein said protein is bound to said delivery vehicle.

In another aspect is provided a composition including a delivery vehicle and a nucleic acid encoding said protein as disclosed herein.

In another aspect is provided a protein including a mitochondrial localization amino acid sequence attached to a base editor fusion protein including a nucleotide base-modifying enzyme and an RNA-guided DNA endonuclease enzyme with modified endonuclease activity.

In another aspect is provided a nucleic acid encoding a protein as disclosed herein.

In another aspect is provided a vector including a nucleic acid as disclosed herein.

In another aspect is provided a composition including a delivery vehicle complexed with a nucleic acid, said nucleic acid encoding a mitochondrial localization amino acid sequence covalently attached to a base editor fusion protein including a nucleotide base-modifying enzyme and an RNA-guided DNA endonuclease enzyme with modified endonuclease activity

In another aspect is provided a pharmaceutical composition including a composition as disclosed herein, a protein as disclosed herein, a nucleic acid as disclosed herein, or a vector of as disclosed herein, and a pharmaceutically acceptable excipient.

In another aspect is provided a method of altering expression of at least one mitochondrial nucleic acid sequence, the method including introducing into an eukaryotic cell the composition as disclosed herein, a protein as disclosed herein, a nucleic acid as disclosed herein, or a vector as disclosed herein, and a single-guide RNA (sgRNA).

In another aspect is provided a method of treating a mitochondrial disorder in a subject in need thereof, the method including administering to said subject an effective amount of a composition as disclosed herein, a protein as disclosed herein, a nucleic acid as disclosed herein, or a vector as disclosed herein, and a single-guide RNA (sgRNA).

In another aspect is provided a kit, including a composition as disclosed herein, a protein as disclosed herein, a nucleic acid as disclosed herein, a vector as disclosed herein, or a pharmaceutical composition as disclosed herein.

### BRIEF DESCRIPTION OF THE DRAWINGS

FIGS. 1A-1B present MitoCRISPR Base Editor BE4COX8. FIG. 1A shows the tructure of original base editor vector BE4 (upper drawing) and modified mito-base editor: BE4COX8 (lower drawing). FIG. 1B shows confocal microscopy pictures of HEK 293T cells transfected with the BE4 construct (top row), 293T cells transfected with the BE4COX8 construct (middle row), and 143B cells transfected with the BE4COX8 construct (bottom row). The cells were stained with anti-Flag (BE4COX8 construct) or anti-His tag (BE4 construct) antibody and the mitochondria are indicated by the staining of Tom20 protein. DAPI indicates the nucleus. Mitochondrial localization was observed for the BE4COX8 construct by overlap of anti-Flag and anti-Tom20 signals, whereas overlap of anti-His tag and DAPI signals indicated nuclear localization for the BE4 construct.

FIG. 2A-2B present MitoCRISPR Base Editor BE4COX8-NES. FIG. 2A shows the structure of modified mito-base editor: BE4-COX8-NES. FIG. 2B shows confocal microscopy pictures of a HEK 293T cell transfected by BE4-COX8-NES stained for the presence of Tom20 (mitochondrial marker), a Flag tag, and further stained with DAPI. Co-localization of mito-base editor protein and mitochondrial Tom20 protein was observed by overlap of anti-Flag and anti-Tom20 signals.

FIG. 3A-3D present MitoCRISPR Base Editor BE4-SOD2^{MTS3′UTR}. FIG. 3A shows the structure of MitoCRISPR Base Editor BE4-SOD2^{MTS3′UTR}. FIGS. 3B-3D show confocal microscopy pictures of HEK 293T cells transfected by BE4-SOD2^{MTS3′UTR} showing co-localization of mito-base editor protein and mitochondrial Tom20 protein. The BE4-SOD2^{MTS3′UTR} was stained with anti-Flag antibody and the mitochondria is indicated by the staining of Tom20 protein. DAPI indicates the nucleus. The anti-Flag and anti-Tom2 signals show very high correlated signal, indicating efficient mitochondrial localization.

FIGS. 4A-4B present MitoCRISPR Base Editor BE4-ATP5F1β-Flag-NES. FIG. 4A shows the structure of modified mito-base editor: BE4-ATP5F1β-Flag-NES. FIG. 4B shows confocal microscopy pictures of HEK 293T cells transfected by BE4-ATP5F1β-Flag-NES showing co-localization of mito-base editor protein and mitochondrial Tom20 protein by overlap of anti-Flag and anti-Tom20 signals.

### DETAILED DESCRIPTION OF THE INVENTION

Provided herein is a base editing technology that localizes to the mitochondrial matrix and targets mitochondrial DNA for site-specific base conversion. Provided herein are proteins (or the encoding nucleic acids) that include a mitochondrial localization amino acid sequence covalently attached to a base editor fusion protein including a nucleotide base-modifying enzyme and an RNA-guided DNA endonuclease enzyme with modified endonuclease activity. These can be incorporated into delivery vehicles or vectors. All the above can be formulated with a pharmaceutically acceptable excipient. All of the above can be included in kits. All of the above disclosed compositions can be used in methods for altering expression in mitochondria and for the treatment of mitochondrial disorders.

### I. DEFINITIONS

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the invention may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogues or modified backbone residues or linkages, which are synthetic, naturally occurring, and non-naturally occurring, which have similar binding properties as the reference nucleic acid, and which are metabolized in a manner similar to the reference nucleotides. In other words, the term “nucleotide analog” as used herein generally refers to a purine or pyrimidine nucleotide that differs structurally from A, T, G, C, or U, but is sufficiently similar to substitute for such “normal” nucleotides in a nucleic acid molecule. As used herein, the term “nucleotide analog” encompasses altered bases, different (or unusual) sugars, altered phosphate backbones, or any combination of these alterations. Examples of such analogues include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analogue nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Nucleotide analogues used herein also include nucleotides having modified 2′ position of the ribose ring. For example, the 2′ position of the ribose ring is substituted by O-methyl, O-alkyl, O-allyl, S-alkyl, S-allyl, or halo group. Mixtures of naturally occurring nucleic acids and analogues can be made; alternatively, mixtures of different nucleic acid analogues, and mixtures of naturally occurring nucleic acids and analogues may be made. In embodiments, the internucleotide linkages in DNA are phosphodiester, phosphodiester derivatives, or a combination of both.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful reactive functional groups (e.g., reactive groups such as bioconjugate or bioconjugate reactive groups) used for conjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc.;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.

The reactive functional groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

A “labeled nucleic acid or oligonucleotide” is one that is bound, either covalently, through a linker or a chemical bond, or noncovalently, through ionic, van der Waals, electrostatic, or hydrogen bonds to a label such that the presence of the nucleic acid may be detected by detecting the presence of the detectable label bound to the nucleic acid. Alternatively, a method using high affinity interactions may achieve the same results where one of a pair of binding partners binds to the other, e.g., biotin, streptavidin. In embodiments, the phosphorothioate nucleic acid or phosphorothioate polymer backbone includes a detectable label, as disclosed herein and generally known in the art.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self 17 hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88. In illustrative embodiments, sgRNA can be detected by a specificalized stem-loop primers. The multiple stem loop structures in SpCas9 RNA provide significant thermostability during reverse transcription (RT) and PCR.

A “nanoparticle,” as used herein, is a particle wherein the longest diameter is less than or equal to 1000 nanometers. The longest dimension of the nanoparticle may be referred to herein as the length of the nanoparticle. The shortest dimension of the nanoparticle may be referred to herein refer as the width of the nanoparticle. Nanoparticles may be composed of any appropriate material. For example, nanoparticle cores may include appropriate metals and metal oxides thereof (e.g., a metal nanoparticle core), carbon (e.g., an organic nanoparticle core) silicon and oxides thereof (e.g., a silicon nanoparticle core) or boron and oxides thereof (e.g., a boron nanoparticle core), or mixtures thereof. In embodiments, the nanoparticle has the shape of a sphere, rod, cube, triangular, hexagonal, cylinder, spherocylinder, or ellipsoid.

An “inorganic nanoparticle” refers to a nanoparticle without carbon. For example, an inorganic nanoparticle may refer to a metal or metal oxide thereof (e.g., gold nanoparticle, iron nanoparticle) silicon and oxides thereof (e.g., a silica nanoparticle), or titanium and oxides thereof (e.g., titanium dioxide nanoparticle). In embodiments, the inorganic nanoparticle is a silica nanoparticle. The inorganic nanoparticle may be a metal nanoparticle. When the nanoparticle is a metal, the metal may be titanium, zirconium, gold, silver, platinum, cerium, arsenic, iron, aluminum or silicon. The metal nanoparticle may be titanium, zirconium, gold, silver, or platinum and appropriate metal oxides thereof. In embodiments, the nanoparticle is titanium oxide, zirconium oxide, cerium oxide, arsenic oxide, iron oxide, aluminum oxide, or silicon oxide. The metal oxide nanoparticle may be titanium oxide or zirconium oxide. The nanoparticle may be titanium. The nanoparticle may be gold. In embodiments, the metal nanoparticle is a gold nanoparticle. In embodiments, the inorganic nanoparticle may further include a moiety which contains carbon.

The term “silica” is used according to its plain and ordinary meaning and refers to a composition (e.g. a solid composition such as a crystal, nanoparticle, or nanocrystal) containing oxides of silicon such as Si atoms (e.g., in a tetrahedral coordination) with 4 oxygen atoms surrounding a central Si atom. Nanoparticles may be composed of at least two distinct materials, one material (e.g., insoluble drug) forms the core and the other material forms the shell (e.g., silica) surrounding the core; when the shell includes Si atoms, the nanoparticle may be referred to as a silica nanoparticle. A silica nanoparticle may refer to a particle including a matrix of silicon-oxygen bonds wherein the longest diameter is typically less than or equal to 1000 nanometers.

A functionalized silica nanoparticle, as used herein, may refer to the post hoc conjugation (i.e. conjugation after the formation of the silica nanoparticle) of a moiety to the hydroxyl surface of a nanoparticle. For example, a silica nanoparticle may be further functionalized to include additional atoms (e.g., nitrogen) or chemical entities (e.g., polymeric moieties or bioconjugate group). For example, when the silica nanoparticle is further functionalized with a nitrogen containing compound, one of the surface oxygen atoms surrounding the Si atom may be replaced with a nitrogen containing moiety.

The term “mitochondrial localization sequence” or “mitochondria targeting signal” and the like refer, in the usual and customary sense, to a short peptide sequence (about 3-70 amino acids long) that directs a newly synthesized proteins to the mitochondria within a cell. It is usually found at the N-terminus and consists of an alternating pattern of hydrophobic and positively charged amino acids to form what is called an amphipathic helix. Mitochondrial localization sequences can contain additional signals that subsequently target the protein to different regions of the mitochondria, such as the mitochondrial matrix. One exemplary mitochondrial localization sequence is the mitochondrial localization sequence derived from Cox8. Another exemplary mitochondrial localization sequence is the mitochondrial localization sequence derived from SOD2.

The term “Cox8” and the like refer, in the usual and customary sense, to cytochrome c oxidase subunit VIII. As used herein, the term refers to both biomolecules having the sequence of Cox8 and truncated and substituted versions thereof, including proteins and nucleic acids encoding the proteins, and to a sequence that performs the function of the cytochrome c oxidase subunit VIII, which function, as known in the art, is coupling of the transfer of electrons from cytochrome c to molecule oxygen, as the terminal step of the respiratory chain. The sequence of Cox8 (GenBank J04823.1) follows:

(SEQ ID NO: 3)
GGCTACGGCTGACCGTTTTTTGTGGTGTACTCCGT
GCCATCATGTCCGTCCTGACGCCGCTGCTGCTGCG
GGGCTTGACAGGCTCGGCCCGGCGGCTCCCAGTGC
CGCGCGCCAAGATCCATTCGTTGCCGCCGGAGGGG
AAGCTTGGGATCATGGAATTGGCCGTTGGGCTTAC
CTCCTGCTTCGTGACCTTCCTCCTGCCAGCGGGCT
GGATCCTGTCACACCTGGAGACCTACAGGAGGCCA
GAGTGAAGGGGTCCGTTCTGTCCCTCACACTGTGA
CCTGACCAGCCCCACCGGCCCATCCTGGTCATGTT
ACTGCATTTGTGGCCGGCCTCCCCTGGATCATGTC
ATTCAATTCCAGTCACCTCTTCTGCAATCATGACC
TCTTGATGTCTCCATGGTGACCTCCTTGGGGGTCA
CTGACCCTGCTTGGTGGGGTCCCCCTTGTAACAAT
AAATCTATTTAAACTTT

In embodiments, a mitochondrial localization sequence derived from Cox8 includes of the amino acid sequence: MSVLTPLLLRGLTGSARRLPVPRAKIHSL (SEQ ID NO: 1). In the embodiments, the mitochondrial localization sequence derived from Cox8 includes an amino acid sequence that is about 50%, 55%, 60%, 65%, 70%, 75%, 80%, 85%, 90% or 95% identical to SEQ ID NO:1. In embodiments, the mitochondrial localization sequence derived from Cox8 is at least 80% identical to SEQ ID NO: 1. In embodiments, the mitochondrial localization sequence derived from Cox8 is at least 90% identical to SEQ ID NO: 1. In embodiments, the mitochondrial localization sequence derived from Cox8 is at least 95% identical to SEQ ID NO: 1.

In embodiments, a mitochondrial localization sequence derived from Cox8 includes the nucleic acid sequence: ATGTCCGTCCTGACGCCGCTGCTGCTGCGGGGCTTGACAGGCTCGGCCCGGCGGCTCCC AGTGCCGCGCGCCAAGATCCATTCGTTG (SEQ ID NO: 2). In the embodiments, the mitochondrial localization sequence derived from Cox8 includes a nucleic acid sequence that is about 50%, 55%, 60%, 65%, 70%, 75%, 80%, 85%, 90% or 95% identical to SEQ ID NO: 2. In embodiments, the mitochondrial localization sequence derived from Cox8 is at least 80% identical to SEQ ID NO: 2. In embodiments, the mitochondrial localization sequence derived from Cox8 is at least 90% identical to SEQ ID NO: 2. In embodiments, the mitochondrial localization sequence derived from Cox8 is at least 95% identical to SEQ ID NO: 2.

The term “SOD2” and the like refer, in the usual and customary sense, to superoxide dismutase 2. As used herein, the term refers to both biomolecules having the sequence of SOD2 and truncated and substituted versions thereof, including proteins and nucleic acids encoding the proteins, and to a sequence that performs the function of binding to the superoxide byproducts of oxidative phosphorylation and to convert them to hydrogen peroxide and diatomic oxygen, which function, as known in the art, is to clear mitochondrial reactive oxygen species (ROS) and confer protection against cell death. The gene sequence of SOD2 (Genbank NG_008729) follows:

(SEQ ID NO: 4)
TCAAGTGTTTTTTTTGTTTTGTTTTTTGTTTGTTT
GTTTGTTTGTTTTTGACACTGAGTCCCACTCTGTC
GCCCAGGCTGGAGTGCAGTGGCGAGATCTCAGCTC
ACTGCAGCTGCCGCCTCCCGGGTTCAAGCGATTCT
TCTGCCTCAGCCTCCCAAGTAGCTGGGACTACAGG
TGCGTGCCACCACGCCTAGCTAATTTTTGTATTTT
TAGTAAAGACAGAGTTTCACCATATTGGCCAGGCT
GGTCTCCAATTCCTGACCTCATGATCCACCTGCCT
CAGCCTCCCAAAGTCCTGGGATTATAGGCGTGAGC
CACTGGGCCCTGCCAATTTTTGTATTTTTAGTAGA
GATGGGGTTTCACCATGTTGGCCAGGCTGATCTCG
ATTTCCCGACCTCAGATGATCCGCCCACCTCGGCC
TCCCAAAGTGTTGGGATTACAGGCGTGAGCCATGG
TGCTGGGCATATACGCAGATTTCTGACTGTGAAGG
GACCAGCACCCCTGACACTGCATCATTCAGGGGTC
AACTGTACATTCATATCCCCCTTACTACCTACTAG
CTCTATGTGTGACCTTGGTCAAGTCACTGTCTTTG
CTTACAGCAGGAATAAAGCATTTGACTTAGTAGCT
AGCAACATGGAGGATCTGCTCAATGATGCCGACTC
CCTTTGCCCTTTATGGACGTAATCAACTCATGATT
ACCTGCGACACATGCCCACTTTATCCCTTTGTTTA
ACAGAACACTTTTGTTCTGAACCAAGCTCCTCCTT
TCCTTTCCAGTAGTTAATGTGTTCTTTGAGGATGC
AATCTAATCGTAATTTTGCCCGAAGTGGGGAAAAA
AATTAAGCTCACATTGAAACCTTAATATAGCTCTA
TAATCATTTTCTCTTTTAGCCTAGATCACTTCATT
TTGGCAATTTAAGTACTACTTTTGGTCCTATTAAC
AATACCACTCTTGCAAAGGACAGGTTACAACCAAT
TCTCAGATTCCTGCCTGGATAAGATTTGTGACCCC
AAATTTTAAAAAAGCAGAAAAAAACCCCACAAACC
ACAGGCAGGAAGAAAGAAAAACAAGAGAATGTAGC
TAAGGTAATTCCATTAATGATCAGTTTATGTCAAA
GCATGTGGAAATAATGCCTTATAGGAGGATATTTA
GCAACAACGATTTTTTTAAACTCATTTTCTCCTGT
AGGTACAGAATTGCAAGTTTTTTCTGACTTCAAAT
GTGTATAGCTGACTGCGTGTGTGTGTGTGTGTGTG
TGTGTGTGTGTGTGTGTGTGTGTGTGTCAGGGTCT
CCTTCTCTTGCCCAGACTGGAGTAAGGTGACATAA
TCATAGCTCATTGCAGCCATGAACTCTTGGGCTCA
AGTGATCCTCCTGCCTTCAGCCTCCTGGCGTAGCT
AGGACTGCAGGCACATGCTACCACACCCAGCTACT
GTTTTTTGGTAGAGACAGAGTCTTGCTATGTTGCC
TAAGCTGGTCTCAAACTCCTGGCCTCAAGCAATCT
TCCCACCTCAGCCTCTCAAAGAGCTGGGATTACAA
GCATGAACCACTGCACCTGGCCCCATACCTGACTA
CATTTCTCAGCTTCCTTTGTGGCTAGGCTGTGCTA
GGTAGCCTTGTACCTAAATTTTGACCAGTGAGCAG
CAAGTGGAATTTGTTGGATGAATCTACCACAAAGG
ATCCTTACAATGGAGATAGTGGGGCCAGGCATGGT
GGCTCATGCCTGTAATCCCAGCACTTTGGGAGGCT
GAGGCAGGCAGATCACTTCAGTCTAGGAGTTCGAG
ACCAGCCTGGCCAACATGGTGAAACCCCATCTCCA
CTAAAAATACAAAAATTAGCCAGGCATGGTGGCAC
GCACCTGTAATCCCAGCTACTCAGGAGGCTAAGGC
AGGAGAATCACTTGAACTCAGGAGGTGGAGGTTGC
AGTGAGCCGAGATCGCACCACTGCACTCCAGCCCA
GCAACAAAGCAAGACTCTTGATTCGGAAAGAAAAA
ATAAAAAAAGGTTGGGGGAGACAGTGGGAGCCCAG
ACTTTTGTCCTTCCCCTTGCCTAGAAGGGAGATGA
GGTTGCTGGTGCTGTGGAAGCTATTATGGACCATG
AGGCAGCTTTGAAGACAGAAAGCCTGCATCCTTGA
TGACTCAATGGAATTTCATCCCAACCCAGGACTGA
CTGCCCCAGATCTTTGTTACATGAGGGAATACACT
CTTCTGTGTTTAAGAAACTGTGGTTGGATCTGTTA
CTGGAATATGAATGCAGTTAATAACTGGCTCAAAA
TGACTTCATCTCAACCCACATTGGTTGCTTCTTCC
AAACAATATATAGGTCTGTACTGTGGTGGGTCTCA
GGATGGCTGTGATGTAGCCTTAGGAAGTTTATCTA
TGGGAAATCCATATTCATGGTGTCCTGATGTTGCA
GAGGACATCCTGAGCTGGCTGGAGTAACTTGGGAC
ACAGGTCAATCGACTGTAATCTAACTTCTGAGGCC
ATTCAGTACCCTCTACAGTGGCCACCTAAAAAAAA
GGCAGCCAGGTGTGGTGGCTCAAGCCTATATAGAT
CCCAGAACTTTGGAAGGCTGAGGTGGGAGGATCAC
TTGAGCCAAGGAGTTTGAGGCCAGCCTGGGCAACA
TGAAGAAACTCTGTTTCTACAAAAAATAAAAAAAA
TTAGCCAGGCATGGTGGTATGCACCTGTAGTTCCA
GCTTCTTGGGAGGCTGAGGTGGAAGAATGACATGA
GCCCAGCAAGTCGCTGCAATCAGCCGTGATCACGC
CGCTGCACTCCAGCCTGGGCGACAAAAAGAAAAAG
AAAACGGAGCCTGTTCACTGGGTGTGGTAGACAAG
GTAAACTTTTCTTTACCTCCCATATCCCACAACCT
TGGATGTGCTCACAGTCATGGTAGTGTTTTGTAAT
GATGTAGCTGATGACAGGTGTGATGTTGGAGATTC
TTCTACCTGACTGCTGCTATCAGTCCTACCAGCCC
CCAACGTTTGGTGCTTGTTCTAAAGGGCATGTCCT
AGGAGTCGCTTTAAACTCTCAAAGTATCACTCTCT
ATACAAACAAGAAGTGCAAGTAAGTAGCCTGAGCT
CAGCCTCCCAATAGGAATATTTCATTATCACTAGA
TCAAGTCTTTCCATTACAATGACTGATCTGTCTCT
GAATCCTGTGGATTCATCCTTCAAAATGCCCTTTT
CTTCCAGTTCTCATAGCTAGTGCCCTAAAAGTGAC
CTGCAGTACCTCCTGCTGAGACGAATGTACCAGCT
TCCTAACTAGCCTGCACTCCCTTCATCCCCCCAAG
TCAGTGCCAGACCACCTTGCCTGAAAAACCACTTT
CAGTGTGTCTCACCTCAGCAGAAATGTTTCTCAGC
TTCCAATTAACAATCACATCAAACCCCTGCTCTTG
TCTGCGTTTTAAGGGTATCTATAGGCCGGGCGCCG
TGGCTCCTACCTGTAATCCCAGCACTTTGGAAGGC
CGAGGCGGGCAGATCACTTGAGGTCAGGCGTTCGA
GACCATCCTGACCAACATAGTGAAACCCCGTCTCT
ACCAAAAATACAAAAAAAAAAAAAATAGTGGGGCG
TGGAGGTGCACGCCTGTAATTCCAGCTACTCGGGA
GGCTGAGGCAGGAGAATCGCTTGAACCCGGGAGGC
AGAGGTTCCAGTGAGCCGACATCGCGACACAGTAC
TCCAGCCTGAGCGACAGAGCGAGGCTGTGTCTCAA
AAATAAATAAATAATAAATTAAAAAAATAAGAGTA
TCTATAACCTGGTCCCAGCCTGAATTTCCTTTTTC
ACCCCAACACGTAGCCCTAGTTACATTCTTCTGAC
GTCTGTAAACAAGCCCAGCCCTTCCTGTTGTGAAG
CCAAGTTCAGGTGGTTCCTCTTCGCCTGACTGTTT
TCCCATTCCACTTACCGGAAGCCTAGTCATCCTTC
GGAGGGCTGTACGGGGGTTGCAAGAAGCAACGGAA
ACGGTTCAGCACCTGCTACCTTCCATCATATTCTT
TTCAATAAAGGGGCAACTCCCGCCAATGGCAGTGT
AGATTTCCTAACCTCTACACATGGAAGATTCACAC
CATTCAGGATTGTTGTTTAACTGTTGAGAGAGCAC
TTGATACTTAACAGCTTACTAGGCTACAAGACAGC
GCAGGAAAGAATCCTCTGTTGTCCTTTTATGTTAT
CCTGAACAGTTGGTTCACAGAGTTACTGTAAACAC
ACAAAACATGACTGCCAGGGCTTAGTAGTGAGGAA
GGTGGGAACTAGTCCTGACTCAGTTAACTGTGCCC
AGGAGAAGCTGCTTAACCTCAAAGGATTTCACTAT
TACTAGAATCAATAATACCAACCCTAGGGGTAAAA
ATAAAGATAAATGTGTGCAAATCCTGCCTGCAGTC
TCGGGCACGTCGTGGGTGTCCAAGAACTGTTCTTA
GGCAGCCGGTGGGGACAAAGTCTGTGTGCCTCCTG
TCCTGGAATAGGTCCCAAGGTCGGCTTACTTGCAA
AGCAAGGGTACGGCGCAAGAGTACTGAATACGGGT
TGGAAGGGCGCTGGCTCTACCCTCAGCTCATAGGC
CGGCTGGGCGGCGCTGACCAGCAGCTAGGCCCCGT
CTTCCCTAGGAACGGCCACGGGGGCCCTGGGAGGG
TATGAATGTCTTTTTGCAGTGAGGCCTCTGGACCC
CGCGGCCCCCCGGCAGCGCAACCAAAACTCAGGGG
CAGGCGCCGCAGCCGCCTAGTGCAGCCAGATCCCC
GCCGGCACCCTCAGGGGCGGAGCCGGAGGCAGGGC
CTTCGGGCCGTACCAACTCCACGGGGGCAGGGGCC
GCCTCCCTTCGGCCGCGCGCCACTCAAGTACGGCA
GACAGGCAGCGAGGTTGCCGAGGCCGAGGCTAGCC
TGCAGCCTCCTTTCTCCCGTGCCCTGGGCGCGGGG
TGTACGGCAAGCGCGGGCGGGCGGGACAGGCACGC
AGGGCACCCCCGGGGTTGGGCGCGGCGGGCGCGGG
GCGGGGCCCGCGGGGGGGGGGGGCGGGGCGGCGGT
GCCCTTGCGGCGCAGCTGGGGTCGCGGCCCTGCTC
CCCGCGCTTTCTTAAGGCCCGCGGGCGGCGCAGGA
GCGGCACTCGTGGCTGTGGTGGCTTCGGCAGCGGC
TTCAGCAGATCGGCGGCATCAGCGGTAGCACCAGC
ACTAGCAGCATGTTGAGCCGGGCAGTGTGCGGGTG
AGAAGAAAGGGGACCCGGTCACGGCCCCAAGGGCG
AAGGGGCTCGCGGCGGGCAGGGCCTCCGCGGCAAT
GGCGACAGTGGCCGCACCGGGCCTGGCGGGACCGG
GGCACCTGCAGGCGGTTCTCCCGGGAGTGCCCGGC
GCGGCGGCTGGAGCGGGGATCCGCAGGGAGGGGAC
GCGGGGACTCGGGGGACGCCGCGCGCTGCCGTTCC
TCGGCAGCCCAGCCTGCGTAGACGGTCCCGCGGCG
CTGACTGACCGGGCTGTGCTTTCTCGTCTTCAGCA
CCAGCAGGCAGCTGGCTCCGGTTTTGGGGTATCTG
GGCTCCAGGCAGAAGCACAGCCTCCCCGACCTGCC
CTACGACTACGGCGCCCTGGAACCTCACATCAACG
CGCAGATCATGCAGCTGCACCACAGCAAGCACCAC
GCGGCCTACGTGAACAACCTGAACGTCACCGAGGA
GAAGTACCAGGAGGCGTTGGCCAAGGGTAGGTTCC
AGGCTGAGCGGCGGGAGGCAGTCCCCGGCAGAGGC
GACCCCAGGGAGCCAGGCCCCATACGGACGGGCCT
CTCCGTGGAGGAGAACTCGCTTCGTATTTGTACCG
GTTCCGAGTTTTCCAGGCACGATAGTCTCTCTTTT
AAACACATGGTCTACCTCATTGTAGAAGGAGTGCC
TCGATGGGTTTGAACACACTTCTGTCATCTCAGGG
AACTTGGGGTCCTGCGAAGGAGCTTGCCTTACTGT
TGTGAGCCACATTCCGTTACACATATTGCCAGCAC
TGGTGAATTGTAGGGCCTGAAAAGAAAGCTCTACT
GTGTCACTCGTTTTTTTTGCAAATTGAAATTGTTC
TTGTTGTATAATGTGCTTTGGGGAAATGTTTGGTC
TCTCAGGTAGGTGTGCCAGCCGTTTGCAGGAGGGC
TGAGAGCGCCTGTCCACTGGTGGCCAGACATCATC
GGGTCCGCAGGTGTCTCTGAGTGTCAGGGTCACCT
CCTGATAGAAGTGGGAGTGGTGTCTTACTGCCAGG
TCACACTGAAGGTGGGAGACAGGAGGACACTACTC
CGTGCTAGGAACCATGGTCCTTGTCATCTTCCTGA
GAGCAAACGGGGTCGGGACTCCAGCCTAGGACTTG
GAGACTCCCTGCATCCCCAGCTACCCTGCAGGTGA
AACTTCACTGAGCCCACTTGACTTCAGGTGGGAGA
GGAGGAGGCACACCGTTGTGGTGTGCGTTACCAAA
AAAGCAGTAACCTAACTGTGGAACAGTTCAGGATC
TTTAGACTTGATACTTTTCTTGATTTCCCTTAGGG
GTCAAAGTTCACAAGGAAAATAGCCCTTTAGTGGG
AAACTGAAACAGGCTTGTTTTTATTTACTATTTAC
TCATATTTGTAATGCAATTAAAATATATGTTGTTA
ACTTTTTTTTTTTTTCTCAAGAGCTCGGTTGATAA
AACCAGGGGTATGTGGACTTTTTGAGTCTGTGCCT
TTTGGGGGTGTGTATGGGAATGTTATGTTTTAACG
TTTTTTACAAAATAGTATTGTCTTTTTAATTTATT
CGTTAGTGGTTTGCACAAGGAAGATAATCGATAGT
CATGTTTTTTAGACTCTCTGTATTGCTTGGTAAGC
TACGTAGTAAAAAATGTTTACTTTTCCTTAAATGT
TTTGAATTTCGGGGTTATGAAATTTGTTGAGTAAT
TTTTAGACAGTCACATCTTGTTGACTGGAGGCATC
TAGTGGAAAAATGCAGTATTTCAGCCTGATTGTGT
TTGAAGTAAATGATTAAAAGAGGAGGAAGTTACCA
CATTCTGGAAGATTTACTTGAGACAGACGAACCTT
GAATTACGGGAAAAGGCCCCGTGATTTAGGAAATA
ACAAATTTGGGAAACATGTAATGGGGAGAGACTGG
GGAATACCCCAGTTGTGAAAGTACTTCCTGTAAGG
CAACATCTGACACCAGGAACCTTTCTCTTCAGTAT
TTTAAAAACAACTTAATTTCAGTCCTTTACTTGTG
GAATCAGAGCCTTACTTATGTAATACAACCCACTG
GAAAAAAGCTTTTTATTGTATTGTACTATATTGTT
TATAAGTGATTGAGTACCTGCAGAGCTTTCTTTTA
CTTAAACATATTTTAAAAATTATTAAAAAGATTTT
CATGTTTGAAAACTTGGGGAAAAAAGATAAAACAA
TGTCATAATCTCATCACATTTAACTGACTAATATG
AATATTTTTCAACAAAAAGCATTTGACTTTTATTA
TTATTTGAGGCAAAAACTAAACTGAAACCAAATAA
AATGTTGAATTTCAGAAAAGTGTTCTTAAAAGCAG
ATATTAAGCCCTCAATTTTTATTAATTTTAAAATG
AGTAAGCAAACTGTAAGATGTTAAGATTATTAAAT
TATCTCTCATTAAGCATGTATCTTTCCCATTTTAA
AAATTCAGTTACTATTGATTTATTAAAAAACAATA
GAAAAGTTACATCTATTTTTAGAGTGACTCTCAAG
AAAATTTAATAAGGGAAACACAGATCTATAAAAAA
CTGTTTGTAAACCTAACAAAGGAAAAAAAAAGTTG
GTGTCTTTAAAAAACCCTTTGAGATTAGCTGTGTA
TTCAGATATCAGTTGGTTCTCTCCTTTCTGAATCG
CTAAACAACTGGAGCTTGTTTCAGAAAGCAGCATT
GTCTACGTATGTTCCTTTTAAAAATTTTTTTTTTA
ATTCTGTCTAAAAAAAACAGACGGGGTCACACTAT
GTTGCCCAGGCTGCTCTTGAATTCCTGGGCTCCCC
TATCCACGCACCTCGGCCTTCCAGAGTGCTGGGAT
TACAGGTGTGAGCCACCGTGCCTAGTCAGACATAC
TTTTTTTTTTTTTTTTTTTTTTGACGGAATCTCAG
TCTGTCCCGCAGGTTGGAGTGCAGTGATGTGATCT
CGGCTCACTACAACCTCCGTCTCCTGGGTTCAAGC
AATTCTCCTGCCTCAGCCTCCTGAGTAGCTGGGAT
TACAGGCGTACGCCACCACGCCTGGCTACTTTTTG
TGGGTTTTTTAGTAGAGACAGGGTTTTTCCATGTC
GTCCAGGCTGGTCTGGAACTCCTGACCTCAAGTGA
TCCGCCCATCTCAGCCTCCCAAAGTGCTGGGATTA
CAGGCGTGAGCCACCTCACCTGGCCTTGTATGTAT
GTTTTTTTCTTTCTCTTTCTTTTTTTTTTTCTGTG
ACGGAGTATTGCCCTGTCGCCCAGGCTGGAGTGCA
GTGGCGTGTTCTTGGTTCACTGCAACCTCTGCCTC
CTGGGTTCAAGCCATTCTCCTGCCTCATGCTCCTG
AGTAGCTGGGATTATAGGCACGCGCCACCATACCT
GGCTAATTTCTGTATTTTTAGTGAAGACAGGGTTT
CACCATGTTGGCCAGGCCGGTCTTGAAATCCTGAC
CTGAAGAGAGCTGCCCGCCTCGGCTTCCCAAAGTG
CTGAGATTACAGGCGTGAGCCACCGCACCCGGCCC
AGACATATGTTCTTAACGTACACTTTCAGAACAGC
AGTTGAGAACTTTTTCCAAAATGTAAGCTCCTTGA
GAGCAAAGCTCTCAGTGGGTCTCTGTGTCCTTTAC
TTTGCTTCTCTTGGTATCTTGTGGATAATATGCAC
AAAATATTTATTAAACAAGAGGATTTCCATTTTTT
TCTAATATTAATAGTTCATTTAGTCTGTAGCTTCT
TGAAGGCATAGAAAAGACTATTTTATTTTGCCTAC
TTATTTGGCAAGGTACCCATGCAGTATGCCAGGAG
TTACAGGGCCTATTGAGAAGGAGTAAAGGGTCTGG
GAGGGGTGGCAGGGAGTTGCTAATTCATGTATGGT
GGCTAGAGAAGGCTTCACTGATGGGGTTGATGTTT
CAGTGGAGACTTGAAGGAGGAACAGGAACGCAGCA
TGAGACTGTCTTGGGGAAGAGTGTTCAGCAGAGGC
AGCAGTGCTCAGCTAGCCTTGAGGGGAACATCCAA
GTTGGTCATGTGAGTGCAAAAAGGCCAGTGAGGCT
GCAGCTGCGTGAGCACGTGCTCAATGACGGGGTCA
AGTGCAGCGCCTCTTGGTAGTAAGAGTAGCTGAGT
GTAAACACCTAGGGTTCGCTCCAGTGAGAAGGGGA
GCCTTTGGAGGGGGCAAGGCCCACCCCCACCCCCC
AAAACTGAGGGGATAGTGGCACCAGTGCAGTGAGA
AATACTGGTCCTCTGTCCCAATGAGGTGGCAGTAG
AGTTAGGGAGAAGGGATTAGGTTCTGCTTGTGTTT
TATTTTTTTCTTTTATTTTTCTCTGTGTGCTTTTA
TATAGATTGGATGTATTTTAAAGGTAGAGCTGGCT
TTGCTAATGGATATGCTGGTGGATTGGATGTGGGG
TGAAGGGAGAGGAGTCAAAAATGACATCAGGTTCC
CAAGGATGTCCTGAGGGACAGAGCTGGCATTTACT
GAGACGTGGAGGACAATAGGAGGAGTAAGTTTGGG
GTAGAACATCAGGAGTTCAATTTTGAGTATGTTGA
GTTTCTTGTTAATGTGACTTGGAGATATTTCCCAA
TGAATCGTCGTCTGTCTTGAAAGTGAGAGTTGAAG
GTTTTCTGTACAGTCTGTATGTTGAGCCATACATT
TTAGCATTCTATTATTTTGATTGTCCTCTTTGGTG
TTTCATTTCCTAAAATCTCTTCTATCTAAGAGATC
CCTGAGCCTAGCAGCCAGGAGTGAATTTTCTAGCT
TTTCCACCTCTGCTAGGTCCTTCATCAGTTGTTCA
GCATGTCTGGGACAAATAATTTTGGGGCGGGGGGG
TGCTGAATTTTAGGACAAACGGATGTTATAGATAA
GCTGGTCCCATTATCTAATAGCTTACAAAGAAAAA
AATATAATGTATACAGTGGTTGAAAAAGTAGGAGT
TACAAAAAAATGTGTTTGCATTTTAACTTTTCAGG
AGATGTTACAGCCCAGATAGCTCTTCAGCCTGCAC
TGAAGTTCAATGGTGGTGGTCATATCAATCATAGC
ATTTTCTGGACAAACCTCAGCCCTAACGGTGGTGG
AGAACCCAAAGGTTGGTATATATTGGTGCACCCTT
ATCTACATTTTGTGCACAGTAGGAATCGATTGTTG
ATTTACTGAAGTTACCTTATACTTGTTTTTTGTTT
TTTTTGTTGTTGTTTTTTTGAGACAAGAGTTTTGC
TCTTGTTGCCCAGGCTGGAGTACAGTGGCGCAGTC
TTGGCTCACTGCAACCTCCGCTTCCTGGGTTCAAG
TGATTCTCCTGCCTCAGCCTCCCAAGTAGCTGGGA
TTACAGGCGCCCGCCACCACGCCTGGCTAATTTTT
GCATTTTTAGTAGAGATGAGGTTTCATTCACCATG
TTGGCCAGGCTGGTCTTGAACTCCTGACCTCATGA
TCTGCCCACCTTGGCCTCCCAAAGTGCTGGGATTA
CAGGCATGAGCCACCGTGCCTGGCCTAAGTTACCT
TATTCTTACCAGAGGTATCATAGTGGCATATTTTT
CATAACAACGAGTATGATAAAAGTTGTAACTTTCA
AAAGGACACAAGTAGGAATGGAGAATGCTTTCATG
GGGTTATGTCATAAGATTTTATGTTCACGCCAACA
TAGAATATCACATCAACTCCACCTTCTGGTTTCTG
GATGGTACTTCATATTAGGGAAAGACTGAGAGACT
TCATTTTATGAACTTTTTTTTTTTGAGACGGAGTC
TTACAGTCGCCCAGGCTGGAGCGCAGTGGTGCAAT
GTCGGCTCATTGCAACCTCCGCCTTTCAGGTTCAT
GCAATTCTTGTGTCTCAGCCACCCAAGTAGCTGGG
ATTACAGGCATGTGGCATCACTCCCGGCTAATTTT
TATATTTTTGGTAGAGACAGGGTTTCACCATGTTG
GCGGGGCTGGTCTCGAACTCCTGGCCTCAAGTGAT
CCACCCACCTTGGCCTCCCAAAGTGCTGTGGTATG
GATGTGAGCCACCGCGCCAGTCTAGAAAAGATGAA
CTTTCTGTCTACTAGTTTCTAGGGCCTGGATTAGA
TAGTTAACCGTCTGTACCTTTTTTCATCTATGTGA
TACAGGGATAACAATGGTGTTGTTTTGAGAATTAA
ATTATAGTGTCATTTGCTATTTTTCACTCTTTCCT
AACTGATAAAAAGTCAGTTGTTCCAGGAAATTAGG
TTTTCTGGGTATTTTTAAAAAGTGTTCAGGGAATG
AGGAGAGTACAGGGCTAGATGGTCTTGTTCGCTAT
CACTGTTTCCTTGACTTCCTGTGTAGCTTGTGGAG
GCCCAGATGACTTTAATTATCTTCTTCACCCAGGG
AGGGCATGAGGCGTAAATGTAGTTCTTGTCTCTTG
TTTCTGGGCTCCGGATATGCACCCTAAGTTTCTGT
GGTTTTATTGTTCATTTTTAATCAGTGTTATGGAA
GGCAGCCTATCTTATGCACTTGAAATAAGCAATTT
CATTTTAAGGTAATGATGAATGAGGAGAGTAAGCT
TGGGAAACTGGGCAACTTTTTCTTTATTTTTGTTT
TTATTTATTATTATTTTGGAGATGGAGTTTAGCTC
TTTCGCCCAGGCTACAGTGAAGTGGCTTGATCTTG
GCTCACTGCAGCCTCTGCCCATCCCGGGTTCAAGT
GATTCTCTGCCTCAGCCTCCCGAGTAGCTGGGATT
ATAGGTGCCTGCCACCATGCCCGGCTAATTTTTTT
GTATTTTTAGTAGAGAGGAGTTTCTCCATGTTGGT
CAGGCTGGTTTTGAACTCCTGACCTCAGGTGATCC
ACCTGTCTCGGCCTCCCAAAGTACTAAGATTACAG
GTGTGAGCCACCGTGCCTGGCCAACTTATTTTTTT
TCTGAGTTCAGTCTTCTAGACTATCGGTTAATACT
TTTGAAGTTTTGTCAGTTACCAGAATATCAGATAT
ATTCATATGCAACCAGTGGTTTTGGTATGCTGCCA
TTTTTGTTTAATCTGTACCACATTCCATCATTTGT
GCTTATAAAGATGATTACTTTTAAACCCGTAGAAT
AAGAGAAGTGAGATTTTGACCCAGTTGATTTAGTT
GATGTGACTAATCTAGAATTATATTCTGTATTACT
ACAGAATAAAAGGATTACAGCTCTTAAAGTATACC
CTTAGTTCAGTCGTAGAGAAAACTGCCCATCAAGA
CAGGAAGAGCTGGGCTGCCTTGGAGAAGAACAATG
ACTTTATTCCCAAATGGAGGCGTAGAAGAAGGAGG
GTAGAAGTCAAAGGAAAATTTTCTATTCAGGGAAA
AGAAAAAGCTAACAGAAAGTAGTATTTCTTTCCTA
ACAAAGATAGTACAGTGAGTGGGGAAATCCCTCTA
ACACGATTCATTAAAAAATGTGAACTACGCAGCTG
GAGGCCATTATCCTAAGCGAATTAACCCAGGAACA
GAAAACCAAATACCACATGTCTCACTTGCAAGTGG
AAGCTAAACATGGAGTACTTATGGATATAAAAATG
GCAGCAACAGACACTGAGGACAGAGCAGGGAGGGA
GAAAGAAGGGGACGGGGGGTGAAAACTGTTGTATA
CTAAGCTCGCTACCTGGGTGATGGCATCAATTGCA
CCCCAGACCTCAGCATCATATGATACCCGTGTAAC
AAACCTGTGCGTGTACCCTCTGAATTTAAAATAAA
ATTTGAACCGGGCACGGTGGCTCACGCCTGTATTC
CCAGCACTTTGGGAGGCCAAGGCGGGTGGATCACC
TGAGGTTGGGAGTTTGAGACCAGCCTGACCAACAT
GGAGAAACCCTGTCTCTACTAAAAACACAAAATTA
GCCGGGCGTGATGGCACGTGCCTGTAATCGCAGCT
GCTCGGGAGGCTAAGGCAGGAGAATTGCTTGAACC
TGGGAGGCAGAGGTTGCAGTGACCCGAGATCATGG
CATTGCACTCCAGCCTGGGCAACAAGAGTGAAACT
CCGTCTCAAAAGGTAAAAAATGAAAAAAAAAAAAA
AAAAAAAAAAAAGTTGAAGTTATAAAAAACAAAAA
CGTGAATTAAAATTGTGAAACTTAGATCCAGGTGT
CGCATTCTGATGTTGTCTAATTTCTTGGGCCCTAT
GACAAAAATATTTTAATACATGTAATATAACATTT
TACTGTAATTATTGAAATCTGTTCATTTGTGGGTG
GTTTTGGATTTTTTTTTTAATAGGGGAGTTGCTGG
AAGCCATCAAACGTGACTTTGGTTCCTTTGACAAG
TTTAAGGAGAAGCTGACGGCTGCATCTGTTGGTGT
CCAAGGCTCAGGTTGGGGTTGGCTTGGTTTCAATA
AGGAACGGGGACACTTACAAATTGCTGCTTGTCCA
AATCAGGATCCACTGCAAGGAACAACAGGTTAGAT
TTAAAAATTGTGATTTCATTTGGGAGAGATGCTCT
ACTGTAAAGCATTCAACTAGAAATAAGGAAAACTA
ACAGTGTTTTAAGAACATGTAATAATTTGCAAATC
TTAACAGATAACACCCAGAGTCTTGTGTAAGTAAA
AATGTTTTAGAAGTCCTGATATTTCATAAAATTAA
GTACACGTAATTTTATAGGTACATTAACAAACATG
TTTTATTTTTATTTATTTTTTTTGAGACAGATCTC
ACTGTTGCCCAGGCTGGAGTGCAGTGGTGCGATCT
CGGTTCACTGCAACCTCCGCCTCCTGGGTTCAAGT
GATTCTCCTGCCTCAGCCTCCCAAATAGCTGGGAT
TACAGGCACATACCACCATGCCCAGCTAATTTTTG
TGTTTTTAGTACAGACAGGGTTTCACCATGTTGGC
CAGGCTGGTCTTGAACTCCTGACCTCAGGTATCTG
CCTGCCTCGGCCTCCTGAAGTGCTGGGATTACAGG
CATGAGCCACCATGCCCAGCCAAATGTTTTACTTA
TTAACCCTGCTTCATTCTACAGAGAATTAATATCA
GCTTAGATAATTATATGTACTAAAATAAAAAAAAA
ATTAGCTATAGAACAAGAGTGAGGAAAAGAAATAC
AAGCAGAGACTGATTTCAGAGCTTGTACTGTTACC
TCCTCTGCTGAGCATTTGAGGTTAAACTGGGACTA
GATCTCAGGTTTCCAGACCTTCTGCCTTTCTGGGG
CTCTTTGGTAGAGTATATGAAAATATTCGGCTTGG
AAGTGCCAGGCTGGGCTCACCAAATACTTTGCTGT
TGAGCCTAATTGGATTTAAAAAATGTCACTGAAGA
GTATGGGAAATAGAGCCATGAGTTGTCTGGCAGAG
GGTTGGGACTTTGAACTTAAAGTGATACCTTCACA
TCAACAACAGTCATTCTCTGCCCCTTCTTCTACCC
CTGACATTGGAATACTCTTGATCTGTGGCTCACTT
CACAGGATAGACCCAGATTTTGGCAGAATCTGAAC
AGCTCTTTTGGGACAGTGACATGGCATAACAATTT
GTAAAAGCCACAAAAAATTTGTAAGCACCGATCTG
TAGAGTCAGGTTATTTGTAGAGCTGCATGACCAGA
AAGGCTGATATTTGTAAGTGAGTCCCAGCTACACT
CCTTCCTCTCCCTGTGGAAAGCACATTAGAAGGAA
AATGAGGGAATGTCATGGCCTTTTGTTTATAAAAA
TCATTAATAAATTTTGCTACTTGAAATTATTAATA
ATAAATTACTAGTGACCTCCCTCACAACTGATCAA
GACATACTGGTTAAGAATTGTTTTTGTAGAGAGAG
CTATAATTTATTTTGAGTGTTTTTGTGTGTGCGTG
ATGGAGTCTCACTCTGTTGCCCAGGCTGGAGTGCA
GTGGCGCAATCTTGGCTCACTACAACTTCCGCCTC
CCAGGTTCAAGCAATTCTCCTGCGTCAGCTTCCTG
AGTAGCTGGGATTACAGGCGCCTGCCACCTCGCCA
GGCTAATTTTTTGTATTTTTAGTAGAGGCGGGGTT
TCACCATGTTGGCCAGGCTGGTCTCGAACGCCTGA
CCTCAAGAGATCTGCCTGCCTCGGCCTCCCAAAGT
GCTGGGATTACAGGCCTGAGCCACTGCGCCCAGAC
CAAAAGTTTTTTTAAATGGATGTTTTACAGATAGA
ACTAAATTCTTCATTTACCTCACTTAATTTCTGAA
AACACTGTGTCCTCCAACTGCTCTGTATTCCCAAA
GATTAAAATTAGCCTTGGGGAGCAGAGCTGGTTCC
TCGTAAACCCCTGTGAGTGCCCAGACCCTTTGGAG
GAGGTTCTCAGTGGGCTTTTGCCTGCTCCCACAGA
TGTAACCCACCCTTCCTGCTGCTCCTAAGACCACC
ATGAGGAAGGCTGGGAATTGGCTCTTGAATTAATT
TGTTGGTATTTTTAAGAATAGCTATTAAAGTACTG
TTGTTAATTTTCCCAAGTCTATTTTATTGTTAGTA
TTTGTCACATTTTCTTTGTAAACAGTGAATTAAAA
ATATTTTATAGGAATATTCATTCTTATCTAGATTT
CCTACTTATATAAAAAAACAAGTGAATGAGATTGT
TACAAAGGGTAATTTTGTGTGAGTAGAATAATAAA
AGTTGAAATTGAGAAGATGCAATGTTTTAGACTGA
AACTGATGGTTGGTTTGTTTTCCCCTTCTTTCTAA
CAGGCCTTATTCCACTGCTGGGGATTGATGTGTGG
GAGCACGCTTACTACCTTCAGTATAAAAATGTCAG
GCCTGATTATCTAAAAGCTATTTGGAATGTAATCA
ACTGGGAGAATGTAACTGAAAGATACATGGCTTGC
AAAAAGTAAACCACGATCGTTATGCTGAGTATGTT
AAGCTCTTTATGACTGTTTTTGTAGTGGTATAGAG
TACTGCAGAATACAGTAAGCTGCTCTATTGTAGCA
TTTCTTGATGTTGCTTAGTCACTTATTTCATAAAC
AACTTAATGTTCTGAATAATTTCTTACTAAACATT
TTGTTATTGGGCAAGTGATTGAAAATAGTAAATGC
TTTGTGTGATTGAATCTGATTGGACATTTTCTTCA
GAGAGCTAAATTACAATTGTCATTTATAAAACCAT
CAAAAATATTCCATCCATATACTTTGGGGACTTGT
AGGGATGCCTTTCTAGTCCTATTCTATTGCAGTTA
TAGAAAATCTAGTCTTTTGCCCCAGTTACTTAAAA
ATAAAATATTAACACTTTCCCAAGGGAAACACTCG
GCTTTCTATAGAAAATTGCACTTTTTGTCGAGTAA
TCCTCTGCAGTGATACTTCTGGTAGATGTCACCCA
GTGGTTTTTGTTAGGTCAAATGTTCCTGTATAGTT
TTTGCAAATAGAGCTGTATACTGTTTAAATGTAGC
AGGTGAACTGAACTGGGGTTTGCTCACCTGCACAG
TAAAGGCAAACTTCAACAGCAAAACTGCAAAAAGG
TGGTTTTTGCAGTAGGAGAAAGGAGGATGTTTATT
TGCAGGGCGCCAAGCAAGGAGAATTGGGCAGCTCA
TGCTTGAGACCCAATCTCCATGATGACCTACAAGC
TAGAGTATTTAAAGGCAGTGGTAAATTTCAGGAAA
GCAGAAGTTAAAGGCAAAATTGTAAATCAGTCGAG
ATCGGGTGCCTTCAGGGTGGTATGGCTGTATACCA
AAATTGTAAATCACTACATGAAGCTTATATATTGG
TTTGGCCTGAAAGGTGAAGTGGGGTAGGCAGGGGG
CGGGCTTACAGGTTATGGTGGATTCAAAGACTCCC
TGATTTGTGATTGGTTAAGGAAGCAAAGCTTTGTC
TAAAAACTTGGGGTCCGCAGAAAGGAACATTAAGG
TCTGGCCAGGCCCCTCAGGAAGAAACTGAGAGCAA
AGAATGGAGGTCAGAGTTTAGTCCCTGGTGTTCCC
CCTTATCTGACGTCTGTGTGAATCCATTTGGTGGG
GGTCTGGGTTTCTGAAAAGTAGCTCAGGGGCACGT
GTTAAGGATGTCTCTAGGTGACTCTAACTTCCCTG
GCTATTGTTTGAAACTGTTATGACCTTCTTGCTTA
TCAGCTTGCTGGTTTCCTTCTCGGGGCGAGCTGGG
TGCCTGGAGTTTTCGGTGAAGGAAACTCAAGATTC
TCCTTTATTTCTGTGCTTGTGGGAATCCCCCTGGC
ACACCCCAAAGAGGGGTCCCTGCTCCGTCTCACAG
GGATCTTTTTGTATATTTGGCTTAGCATCATACAT
TTGCCATGTTGTTTCATCATCTGCCTAATTTACTG
TTTTTGAATATTTCATTTGTTTCTAATTGTTACTA
CAGATAATGCTGGGGTGAGCAACTCTGTGTACATA
GGTTTATCTCCTATTGGAATATTTTCTTTATATAG
GCGTTTTTTTTTTTTCTTTTTTTTTGGAGACAGAG
TCTTGCTCTGTTGCCCAGGCTGGAGTGCAGTGGCG
CGACCGGAGCTCACTGCAACCTCCACTTCCCGGGT
TCAAGTGATTGTCCCACCTCAGCCTCCTGAATAGC
TGGGATTACAGGTGCATGCTACCATGCCTGGCTAC
TTTTTGTATTTTTAGCAGAGACAGGGTTTCACCAT
GTTGGCCAGGGTGGTCTCGAACTCCTGACCTCAAG
TGATCCGTCTGGCTCAGCCTCCCAAAGTGCTGGGA
TTACAGGTGTGAGCCACTGCACCTGGCCTATATAG
GCTTTTTTCTTAAACCTATTTAGTAATGTTTTCCC
AAGTTTATTTTTTATTTTTAATTTTTTCCCCAAGT
TTATTTTTCTATTTTTTTTTCATGGAAAAATGGGG
TAACTTAGCAGTTTCAATATTGAAGACTGAAGTTT
AAAAAAAATTTAAATTCAAGGTACTTTTAAAATTC
AGTTAGAAAAGTAGGCTTTAAAAATTATTAGAGAC
AAGAGTACCAAAGCGGTGTGTGTATGTGTGTGTGT
GTATGCATGCTTGTGGATTGGAAAAACTTTGGAGA
CTGATTACTTTTCATTATATATGTGTCACAGTGAA
ACAGCTTTTATGTGTCATGTAAGATTACTGCTTGC
CTCTCTAAGGAAGGTCGTGACTGTTTAAATAGACG
GGCAAGGTGGAACCTTTTGAAAGATGAGCTTTTGA
ATATAAGTTGTCTGCTAGATCATGGTTTGTATTGA
ACTAACAAGGTTTGCAGATCTGCTGACTTATATAA
AGCTTTTTGATTCCTACTAAGCTTTAAGATTTAAA
AAATGTTCAATGTTGAAATTTCTGTGGGGCTCTAT
TTTTGCTTTGGCTTTCTGGTGAGAGAGTGAGGAAG
CATTCTTTCCTTCACTAAGTTTGTCTTTCTTGTCT
TCTGGATAGATTGATTTTAAGAGACTAAGGGAATT
TACAAACTAAAGATTTTAGTCATCTGGTGGAAAAG
GAGACTTTAAGATTGTTTAGGGCTGGGCGGGGTGA
CTCACATCTGTAATCCCAGCACTTTGGGAGGCCAA
GGCAGGCAGAACACTTGAAGGAGTTCAAGACCAGC
GTGGCCAACGTGGTGAAACCCTGTCTCTACTAAAA
ATACAAAAATTGTTTAGCTCTGTTTTTCATAATAG
AAATAGAAAAGGTAAAATTGCTTTTCTTCTGAAAA
GAACAAGTATTGTTCATCCAAGAAGGGTTTTTGTG
ACTGAATCAGCAGTGCCTGCCCTAGTCATAGCTGT
GCTTCAAAAACCTCAGCATGATTAGTGTTGGAGCA
AAACAAGGAAGCAAAGCAAATACTGTTTTTGAAAT
TCTATCTGTTGCTTGAACTATTTTGTAATAATTAA
ACTTTGATGTTGAGAAATCACAACTTTATTGTACA
CTTCATTGCAACTTGAAATTCATGGTCTTAAAGTG
AGATTTGAATTTCTATTGAGCGCCTTTAAAAAAGT
AATACCAAACCATAAAGTTAAAATCTATGTATATT
GAGTCATATCTAAAACCACGTATAAACATAAATTG
TATTTCCTGTTTTAATTCCAGGGGAAGTACTGTTT
GGGAAAGCTATTATTAGGTAAATGTTTTACAAATT
ACTGTTTCTCACTTTCAGTCATACCCTAATGATCC
CAGCAAGATAATGTCCTGTCTTCTAAGATGTGCAT
CAAGCCTGGTACATACTGAAAACCCTATAAGGTCC
TGGATAATTTTTGTTTGATTATTCATTGAAGAAAC
ATTTATTTTCCAATTGTGTGAAGTTTTTGACTGTT
AATAAAAGAATCTGTCAACCATCAAAGAGGTCTGC
ATTATGCTTGCATGTCAAAAACTTTAAAAATCCTA
TAATCTTCTGTCATTTTCACTGAGTTTCCATGGGA
AAGGAATAGTAAATAATGGGTAGTTGAAATATTAC
TCTTAAGACCAAGACCTGTATCTCCAGTCATATCT
GTAATAACATCATCTGATAACCTAAAAGCATAGTA
TTAGGGATATACGACAAAACCAAAGTGTTTTTGCT
GTTGTCACATACCACTCAATACTTTTACACCAGGT
TGTCCAGTGGACACCAGCTGGATGTCCTATAATTC
AATTCTGACACTGTCTACCTGGAATTGGAGTCAGA
TCCCACAGGTGGAGGGCTCAGCCCACAAGACTGCC
CCCACTCCAGATACCAATCGCAAGTCCCTATACTT
CTGACTGGCTATAAAGTGGGGTTCCCGTGACCCTT
TCCTTGGTTTTGATTTATTTGCTAGAGCAGCTCCC
AGAACACAGAGAAACACTCTACTTATGTTTACCCA
TTTTTAATTTTATTACTTTATTAAAACTACATATT
GGTTACAATACTAATTTATTGTAAAGGATATTTCA
TTTATTTATTTTTTTATTTAGAGATGGAGTCTCAC
TGTGTCACCCAGGCTGGAGTGCGGTGGCACAGTCT
CAACTCACTGCAACCTCCACCTCCTGAGTTCAAGC
GATTCTCATGCCTCAGCCTCCCAAGTAGCTGGGAT
TACAAGTGTGCACCACCATGCCCAGCTAATTTTTG
TATTTTTAGTAGAGATGGGGTTTCACTCTGTTGGC
CAGGCTGGTCTCAAACTCCTGACCTCAAGTCATCC
ACCCACCTCAGCCTCCCAAAGTGCTGAGATTACAG
GTGTGAGCCATTGCCTCTGGCTTGTAAAGGATATT
TCAAAGAATACAGATGAACAGCCAGATGGAAGAGA
TGCACAGGGCAAGGCATGTGGGAAGGGGTATGGAG
CTTTCATGCTCTCTCCACGTCATCAGCTACCCAGA
AGCTCATCTGGACCCCGACCCTTTGGGGTTTTACG
GAGGCTTCATTATGTCAGCATTACCATTGGCAATT
GGCGATTAACTCAACCTTCAGCCTCTCTCCCCTTT
CTGGAGGTTGGGGGTTGAGGCTGGGAGTCCAAACC
CTCCAATCTTGCCTTGGTCTTTCTGGTAAGCAGCC
CCCCGTCCTGAAGCTACCTAGAGGCTCCCAGCCAC
CAATCATTTCATTGGCAAACCAAAGATACTTCAAG
GGGTTTAGGAGCCAAGAGACCAAGCAAGGATTGAG
ACAATATATATATTTCTTATAAATAACATGATCAC
AAATAGTATTTTATATATTTCTCATAATAGCTGGT
TGTGGTTACTCACACCTATCATCCCAGCTACTAAG
AAGCTAAGGCAGGAGGATCACAGGCCCAGGAGTTT
GCAGCTGTAGTGAGCTATGATTGTGCTACTGCACT
CCAGCCTGGGTGACAGTGTGAGACCCTGTCTCTTA
AAGTTCTTGTAACATCTTAGAACATTAACCAACCA
CAGATTATTGCCTTAACTCTGAAAAATAAAGTAAT
CATACTTTTACAAGATGTATTTTACATAAAGTTCT
GGTGATATTTTACTCTGGTCATTGGAATATAGGAG
CTTACCACCTGGTACCACTCTTCCTAAAGTGGTAT
ACTATTTGGTATAGTACTGGCATACTATTTGGAAA
GGGATGTGAAGTTGGCATGACCTGTCTAGGGTGAA
GTCATGCTGTCTGCTATTCATTGTGGGCTTTACAG
TAATGAGAGTGCTTCTGTTGTCAAGATGCTTCTGG
GACACGTTAAAAAAAATTTAAGATTTCTCAGTTTG
TGAAAAGGAAGGAGCACGAATGCTTTCAAGTTCCT
ATGACATGCTAGTCATCTTTCAGTGTGTCCCATTG
TTGCACCCAGTAGTCTCGATGCCCAGCGTCACGGT
GATTCCAAGTTTGCCTCTCTGCCCACTATAATTTG
TGCTCCATGAAGTCCCAGGACATGAGGTTCT.

The above SOD2 DNA sequence is transcribed into the SOD2 mRNA transcript variant 1 (NM_000636).

The amino acid sequence of SOD2 (superoxide dismutase [Mn], mitochondrial isoform A precursor) as encoded by the SOD2 sequence above (NG_008729) follows: MLSRAVCGTSRQLAPVLGYLGSRQKHSLPDLPYDYGALEPHINAQIMQLHHSKHHAAYVN NLNVTEEKYQEALAKGDVTAQIALQPALKFNGGGHINHSIFWTNLSPNGGGEPKGELLEAIK RDFGSFDKFKEKLTAASVGVQGSGWGWLGFNKERGHLQIAACPNQDPLQGTTGLIPLLGID VWEHAYYLQYKNVRPDYLKAIWNVINWENVTERYMACKK (NP_001019636) (SEQ ID NO: 5). (The mitochondrial localization sequence of SOD2 is

MLSRAVCGTSRQLAPVLGYLGSRQKHSLPD)

In embodiments, a mitochondrial localization sequence derived from SOD2 includes of the amino acid sequence: MLSRAVCGTSRQLAPVLGYLGSRQKHSLPD (SEQ ID NO: 6). In the embodiments, the mitochondrial localization sequence derived from SOD2 includes an amino acid sequence that is about 50%, 55%, 60%, 65%, 70%, 75%, 80%, 85%, 90% or 95% identity to SEQ ID NO: 6. In embodiments, the mitochondrial localization sequence derived from SOD2 is at least 80% identical to SEQ ID NO: 6. In embodiments, the mitochondrial localization sequence derived from SOD2 is at least 90% identical to SEQ ID NO: 6. In embodiments, the mitochondrial localization sequence derived from SOD2 is at least 95% identical to SEQ ID NO: 6.

In embodiments, a mitochondrial localization sequence derived from SOD2 includes the nucleic acid sequence: ATGTTGAGCCGGGCAGTGTGCGGCACCAGCAGGCAGCTGGCTCCGGTTTTGGGGTATCT GGGCTCCAGGCAGAAGCACAGCCTCCCCGAC (SEQ ID NO: 7). In the embodiments, the mitochondrial localization sequence derived from SOD2 includes a nucleic acid sequence that is about 50%, 55%, 60%, 65%, 70%, 75%, 80%, 85%, 90% or 95% identity to SEQ ID NO: 7. In embodiments, the mitochondrial localization sequence derived from SOD2 is at least 80% identical to SEQ ID NO: 7. In embodiments, the mitochondrial localization sequence derived from SOD2 is at least 90% identical to SEQ ID NO: 7. In embodiments, the mitochondrial localization sequence derived from SOD2 is at least 95% identical to SEQ ID NO: 7.

In embodiments, the SOD2 3′UTR nucleotide sequence includes: ACCACGATCGTTATGCTGAGTATGTTAAGCTCTTTATGACTGTTTTTGTAGTGGTATAGA GTACTGCAGAATACAGTAAGCTGCTCTATTGTAGCATTTCTTGATGTTGCTTAGTCACTT ATTTCATAAACAACTTAATGTTCTGAATAATTTCTTACTAAACATTTTGTTATTGGGCAA GTGATTGAAAATAGTAAATGCTTTGTGTGATTGA (SEQ ID NO: 8). In embodiments, the SOD2 3′UTR nucleotide sequence derived from SOD2 includes a nucleic acid sequence that is about 50%, 55%, 60%, 65%, 70%, 75%, 80%, 85%, 90% or 95% identical to SEQ ID NO: 8. In embodiments, the 3′ UTR is at least 80% identical to SEQ ID NO: 8. In embodiments, the 3′ UTR is at least 90% identical to SEQ ID NO: 8. In embodiments, the 3′ UTR is at least 95% identical to SEQ ID NO: 8.

A “nuclear localization sequence” or “nuclear localization signal (NLS)” is a peptide that directs proteins to the nucleus. In embodiments, the NLS includes five basic, positively charged amino acids. The NLS may be located anywhere on the peptide chain.

A “nuclear export signal” or “NES” refers to a short target peptide containing 4 hydrophobic residues in a protein that targets it for export from the cell nucleus to the cytoplasm through the nuclear pore complex using nuclear transport. It has the opposite effect of a nuclear localization signal, which targets a protein located in the cytoplasm for import to the nucleus. The NES is recognized and bound by exportins. NESs are peptides that are 8-15 residues long and conform loosely to the widely used traditional consensus of Φ1-X_{2,3}-Φ2-X_{2,3}-Φ3-X-Φ4, where don represents Leu, Val, Ile, Phe, or Met and X can be any amino acid. Extensive databases of nuclear export signals are available (see e.g. Xu et al, Mol Biol Cell. 2012 Sep. 15; 23(18): 3677-3693).

A “mitochondrial import sequence” or “mitochondrial RNA import sequence” refers to an RNA sequence (e.g. small RNAs) capable of directing importation of exogenous RNA to mitochondria. The mitochondrial import sequence may be isolated from or derived from the mitochondrial transcriptome. Exemplary mitochondrial import sequences include, but are not limited to, the 5S ribosomal RNA, RNaseP (“RNP”) and MRP RNA components and a modified γ domain of 5S rRNA, including D loop, F loop, MRP loop, RNP loop, and γ 5 s loop. Detailed description of small RNAs as mitochondrial import sequences can be found in Schneider et al., Annu Rev Biochem., 2011, 80:1033-53; Wang et al., Cell, 2010, 142(3):456-67; Comte et al., Nucelic Acids Res, 2013, 41(1):418-33; Tonin et al., J Biol Chem, 2014, 289(19): 13323-34; Smironov et al., RNA, 2008, 14(4):749-59; and Zelenka et al., J Bioenerg Biomembr, 2014, 46(2):147-56, contents of each of which are incorporated herein as entireties.

For specific proteins described herein (e.g., dCas9 and the like), the named protein includes any of the protein's naturally occurring forms, or variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference or functional fragment or homolog thereof.

Thus, a “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In embodiments, the Cas9 protein is substantially identical to the protein identified by the UniProt reference number Q99ZW2 or a variant or homolog having substantial identity thereto. Cas9 refers to the protein also known in the art as “nickase”. In embodiments, Cas9 is an RNA-guided DNA endonuclease enzyme that binds a CRISPR (clustered regularly interspaced short palindromic repeats) nucleic acid sequence. In embodiments, the CRISPR nucleic acid sequence is a prokaryotic nucleic acid sequence. In embodiments, the Cas9 nuclease from Streptococcus pyogenes is targeted to genomic DNA by a synthetic guide RNA consisting of a 20-nt guide sequence and a scaffold. The guide sequence base-pairs with the DNA target, directly upstream of a requisite 5′-NGG protospacer adjacent motif (PAM), and Cas9 mediates a double-stranded break (DSB) about 3-base pair upstream of the PAM. In embodiments, the CRISPR nuclease from Streptococcus aureus is targeted to genomic DNA by a synthetic guide RNA consisting of a 21-23-nt guide sequence and a scaffold. The guide sequence base-pairs with the DNA target, directly upstream of a requisite 5′-NNGRRT protospacer adjacent motif (PAM), and Cas9 mediates a double-stranded break (DSB) about 3-base pair upstream of the PAM.

The term “Cas9 variant” refers to proteins that have at least 80%, 85%, 90%, 95%, 96%, 97%, 98%, 99% or 100% amino acid sequence identity across the whole sequence or a functional portion of the sequence (e.g. a 50, 100, 150 or 200 continuous amino acid portion) compared to wild-type Cas9 protein and have one or more mutations that increase its binding specificity to PAM compared to wild-type Cas9 protein. Exemplary Cas9 variants are listed in the Table 2 below.

TABLE 2

| Cas9 Variants | PAM domains | References |
|---|---|---|
| Strep pyogenes (Sp) | NGG | Hsu et al. |
| Cas9 | | 2014 Cell |
| Staph aureus (Sa) | NNGRRT or NNGRR | Ran et al. |
| Cas9 | NNGGGT, NNGAAT, NNGAGT (Zetsche) | 2015 Nature |
| SpCas9 VQR mutant | NGAG > NGAT = NGAA > NGAC | Kleinstiver et al. |
| (D1135V, R1335Q, | NGCG | 2015 Nature |
| T1337R) | | |
| SpCas9 VRER | NGCG | Kleinstiver et al. |
| mutant | | 2015 Nature |
| (D1135V/G1218R/ | | |
| R1335E/T1337R) | | |
| SpCas9 D1135E | NGG, greater fidelity, less | Kleinstiver et al. |
| | cutting at NAG and NGA sites | 2015 Nature |
| eSpCas9 1.1 mutant | NGG | Slaymaker et al. |
| (K848A/K1003A/ | | Science 2015 |
| R1060A) | | |
| SpCas9 HF1 | NGG | Kleinstiver et al. |
| (Q695A, Q926A, | | 2016 Nature |
| N497A, R661A) | | |
| AsCpf1 | TTTN (5′ of sgRNA) | Zetsche et al. |
| | | 2015 Cell |

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “RNA-guided DNA endonuclease enzyme with modified endonuclease activity” and the like refer, in the usual and customary sense, to a RNA-guided DNA endonuclease enzyme that has been modified to render the endonuclease unable to create a double-stranded break by inactivating one or both of the catalytic domains that cleave the target or non-target strands of DNA. For example, Cas9 mediates a double-stranded break (DSB) about 3-base pair upstream of the PAM. Examples of an RNA-guided DNA endonuclease enzyme lacking endonuclease activity include, but are not limited to, variants of CAS9 such as dead CAS9 (dCAS9) and nCAS9. “Nickase” mutants of the RNA-guided DNA endonuclease enzyme with modified endonuclease activity, which cleave only one strand. Examples include SpCas9 nickases such as the D10A mutation, which inactivates the RuvC domain, so this nickase cleaves only the target strand. Conversely, the H840A mutation in the HNH domain creates a non-target strand-cleaving nickase.

The term “3-UTR” and the like refer, in the usual and customary sense, to the three prime untranslated region (3′-UTR) is the section of messenger RNA (mRNA) that immediately follows the translation termination codon. The 3′-UTR often contains regulatory regions that post-transcriptionally influence gene expression.

The term “SOD2 3′UTR” and the like refer, in the usual and customary sense, to the three prime untranslated region (3′-UTR) of the SOD2 mRNA that immediately follows the translation of the translation termination codon, and variants thereof.

The term “nucleotide base-modifying enzyme” and the like refer, in the usual and customary sense, to enzymes capable of modifying specific nucleotides. Examples of a nucleotide base-modifying enzymes include, but are not limited to cytidine deaminase or adenosine deaminase, which are capable of the deamination (removal of an amino group) from a cytidine or adenosine, respectively.

The term “a base editor fusion protein comprising a nucleotide base-modifying enzyme and an RNA-guided DNA endonuclease enzyme with modified endonuclease activity” and the like refer, in the usual and customary sense, to fusion proteins of enzymes capable of modifying specific nucleotides to RNA-guided DNA endonuclease enzyme with modified endonuclease activity as defined above. Examples of such fusion proteins include, but are not limited to, those listed in the Table below.

| Name of Original Base | Nuclease (nCas9, | Nucleotide | |
|---|---|---|---|
| Editor construct | dCas9, dCpf1, etc.) | conversion | Citation |
| pCMV_ABEmax_P2A_GFP | nCas9 | A:T-to-G:C base editing | Koblan et al Nat Biotechnol. 2018 May |
| | | | 29. pii: nbt.4172. doi: |
| | | | 10.1038/nbt.4172. |
| pCMV- | nCas9 | A:T-to-G:C base editing | Gaudelli et al Nature. 2017 Nov. 23; |
| ABE7.10 | | | 551(7681): 464-471. doi: |
| | | | 10.1038/nature24644. |
| | | | Epub 2017 Oct. 25. |
| pCMV-SaBE4- | nCas9 | C:G-to-T:A base editing | Komor et al Sci Adv. 2017 Aug. 30; |
| Gam | | | 3(8): eaao4774. doi: |
| | | | 10.1126/sciadv.aao4774. |
| | | | eCollection 2017 August |
| pCMV-BE4- | nCas9 | C:G-to-T:A base editing | Komor et al Sci Adv. 2017 Aug. 30; |
| Gam | | | 3(8): eaao4774. doi: |
| | | | 10.1126/sciadv.aao4774. |
| | | | eCollection 2017 August |
| pCMV-BE4 | nCas9 | C:G-to-T:A base editing | Komor et al Sci Adv. 2017 Aug. 30; |
| | | | 3(8): eaao4774. doi: |
| | | | 10.1126/sciadv.aao4774. |
| | | | eCollection 2017 August |
| YEE-BE3 | nCas9 | C:G-to-T:A base editing | Kim et al Nat Biotechnol. |
| | | | 2017 Feb. 13. doi: |
| | | | 10.1038/nbt.3803. |
| pCMV-BE3 | nCas9 | C:G-to-T:A base editing | Komor et al Nature. |
| | | | 2016 Apr. 20. doi: |
| | | | 10.1038/nature17946. |
| pCMV-BE2 | dCas9 | C:G-to-T:A base editing | Komor et al Nature. |
| | | | 2016 Apr. 20. doi: |
| | | | 10.1038/nature17946 |
| pCMV-BE1 | dCas9 | C:G-to-T:A base editing | Komor et al Nature. |
| | | | 2016 Apr. 20. doi: |
| | | | 10.1038/nature17946 |
| hyeA3A- | nCas9 | C:G-to-T:A base editing | Zhang, X. et al. Nat. Cell Biol. |
| BE4max | | | https://doi.org/10.1038/s41556- |
| | | | 020-0518-8 (2020). |

The terms “single guide RNA,” “single guide RNA sequence,” “chimeric RNA,” “chimeric guide RNA,” “guide RNA”, and “synthetic guide RNA” are used interchangeably and refer to the polynucleotide sequence including the crRNA sequence and optionally the tracrRNA sequence. The crRNA sequence includes a guide sequence (i.e., “guide” or “spacer”) and a tracr mate sequence (i.e., direct repeat(s)”). The term “guide sequence” refers to the sequence that specifies the target site.

In general, a guide sequence is any polynucleotide sequence having sufficient complementarity with a target polynucleotide sequence to hybridize with the target sequence (i.e., a mitochondrial DNA target sequence) and direct sequence-specific binding of a CRISPR complex to the target sequence (i.e., the mitochondrial DNA target sequence). In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any mitochondrial DNA (mtDNA) target sequence. The term “mitochondrial DNA (mtDNA) target sequence” refers, in the usual and customary sense, to a nucleic acid sequence within the mitochondrial genome to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A guide sequence (spacer) may comprise any polynucleotide, such as DNA or RNA polynucleotides. A target sequence may comprise any polynucleotide, such as DNA or RNA polynucleotides.

In general, a tracr mate sequence includes any sequence that has sufficient complementarity with a tracr sequence (i.e., a tracrRNA sequence) to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin.

Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. Where the tracrRNA sequence is less than 100 (99 or less) nucleotides in length the sequence is one of 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length.

The term “transfecting,” “transfection” and the like refer, in the usual and customary sense, to the process of introducing nucleic acids into cells.

The compositions described herein can be purified. Purified compositions are at least about 60% by weight (dry weight) the compound of interest. Preferably, the preparation is at least about 75%, more preferably at least about 90%, and most preferably at least about 99% or higher by weight the compound of interest. Purity is measured by any appropriate standard method, for example, by High-performance liquid chromatography, polyacrylamide gel electrophoresis.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

A “pharmaceutical composition” is a formulation containing the nucleic acids described herein in a form suitable for administration to a subject. In embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed nucleic acid) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In embodiments, the active nucleic acid is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient. A thorough discussion of pharmaceutically acceptable excipients is available in REMINGTON'S PHARMACEUTICAL SCIENCES (Mack Pub. Co., N.J. 1991). Pharmaceutically acceptable excipients in therapeutic compositions may contain liquids such as water, saline, glycerol and ethanol. Additionally, auxiliary substances, such as wetting or emulsifying agents, pH buffering substances, and the like, may be present in such vehicles.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The terms “subject,” “individual,” and “patient” are used interchangeably herein to refer to a vertebrate, preferably a mammal, more preferably a human. The mammal can be e.g., a human or appropriate non-human mammal, such as primate, mouse, rat, dog, cat, cow, horse, goat, camel, sheep or a pig. The subject can also be a bird or fowl. Tissues, cells and their progeny of a biological entity obtained in vivo or cultured in vitro are also encompassed. As used herein, a “subject in need thereof” or “a patient” may be a subject having a mitochondria disease.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal).

As used herein, “mitochondrial disorders” related to disorders which are due to abnormal mitochondria such as for example, a mitochondrial genetic mutation, enzyme pathways etc. Examples of disorders include and are not limited to: loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection. The mitochondrial abnormalities give rise to “mitochondrial diseases” which include, but not limited to: AD: Alzheimer's Disease; ADPD: Alzheimer's Disease and Parkinsons's Disease; AMDF: Ataxia, Myoclonus and Deafness CIPO: Chronic Intestinal Pseudoobstruction with myopathy and Opthalmoplegia; CPEO: Chronic Progressive External Opthalmoplegia; DEAF: Maternally inherited DEAFness or aminoglycoside-induced DEAFness; DEMCHO: Dementia and Chorea; DMDF: Diabetes Mellitus & DeaFness; Exercise Intolerance; ESOC: Epilepsy, Strokes, Optic atrophy, & Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS Kearns Sayre Syndrome LDYT: Leber's hereditary optic neuropathy and DYsTonia; LHON: Leber Hereditary Optic Neuropathy; LIMM: Lethal Infantile Mitochondrial Myopathy; MDM: Myopathy and Diabetes Mellitus; MELAS: Mitochondrial Encephalomyopathy, Lactic Acidosis, and Stroke-like episodes; MEPR: Myoclonic Epilepsy and Psychomotor Regression; MERME: MERRF/MELAS overlap disease; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; RTT: Rett Syndrome; SIDS: Sudden Infant Death Syndrome.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

### II. COMPOSITIONS

In a first aspect, there is provided a composition including a protein including a mitochondrial localization amino acid sequence covalently attached to a base editor fusion protein that includes a nucleotide base-modifying enzyme and an RNA-guided DNA endonuclease enzyme with modified endonuclease activity.

In embodiments, the mitochondrial localization amino acid sequence is covalently attached to a base editor fusion protein. In embodiments, the first moiety (e.g., a protein comprising a mitochondrial localization amino acid sequence attached to a base editor fusion protein comprising a nucleotide base-modifying enzyme and an RNA-guided DNA endonuclease enzyme with modified endonuclease activity) is non-covalently attached to the second moiety on the nanoparticle through a non-covalent chemical linker or covalent chemical linker formed by a reaction between a component of the first moiety (e.g., a protein comprising a mitochondrial localization amino acid sequence attached to a base editor fusion protein comprising a nucleotide base-modifying enzyme and an RNA-guided DNA endonuclease enzyme with modified endonuclease activity) and a component of the second moiety on the delivery vehicle (e.g., nanoparticle or lipid particle). In embodiments, the first moiety (e.g., a protein comprising a mitochondrial localization amino acid sequence attached to a base editor fusion protein comprising a nucleotide base-modifying enzyme and an RNA-guided DNA endonuclease enzyme with modified endonuclease activity) includes one or more reactive moieties, e.g., a covalent reactive moiety, as described herein (e.g., alkyne, azide, amine, ester, N-hydroxy-succinimide, maleimide or thiol reactive moiety). In embodiments, the first moiety (e.g., a protein comprising a mitochondrial localization amino acid sequence attached to a base editor fusion protein comprising a nucleotide base-modifying enzyme and an RNA-guided DNA endonuclease enzyme with modified endonuclease activity) includes a linker (e.g., first linker) with one or more reactive moieties, e.g., a covalent reactive moiety, as described herein (e.g., alkyne, azide, amine, ester, N-hydroxy-succinimide, maleimide or thiol reactive moiety). In embodiments, the delivery vehicle (e.g., nanoparticle or lipid particle) includes one or more reactive moieties, e.g., a covalent reactive moiety, as described herein (e.g., alkyne, azide, amine, ester, N-hydroxy-succinimide, maleimide or thiol reactive moiety). In embodiments, the delivery vehicle (e.g., nanoparticle or lipid particle) includes a linker with one or more reactive moieties, e.g., a covalent reactive moiety, as described herein (e.g., alkyne, azide, amine, ester, N-hydroxy-succinimide, maleimide or thiol reactive moiety). In embodiments, the first moiety is a peptide comprising a mitochondrial localization amino acid sequence that is non-covalently attached to the second moiety that is a base editor fusion protein comprising a nucleotide base-modifying enzyme and an RNA-guided DNA endonuclease enzyme with modified endonuclease activity through a non-covalent chemical linker or covalent chemical linker formed by a reaction between a component of the first moiety and a component of the second moiety. In embodiments, the peptide comprising a mitochondrial localization amino acid sequence and/or the base editor fusion protein comprising a nucleotide base-modifying enzyme and an RNA-guided DNA endonuclease enzyme with modified endonuclease activity includes one or more reactive moieties, e.g., a covalent reactive moiety, as described herein (e.g., alkyne, azide, amine, ester, N-hydroxy-succinimide, maleimide or thiol reactive moiety). In embodiments, the peptide comprising a mitochondrial localization amino acid sequence and/or the base editor fusion protein comprising a nucleotide base-modifying enzyme and an RNA-guided DNA endonuclease enzyme with modified endonuclease activity includes a linker (e.g., first linker) with one or more reactive moieties, e.g., a covalent reactive moiety, as described herein (e.g., alkyne, azide, amine, ester, N-hydroxy-succinimide, maleimide or thiol reactive moiety).

In embodiments, the mitochondrial localization amino acid sequence/base editor fusion protein is a fusion protein. In embodiments, the mitochondrial localization amino acid sequence is N-terminal to the base editor fusion protein including a nucleotide base-modifying enzyme and an RNA-guided DNA endonuclease enzyme with modified endonuclease activity. The fusion of the mitochondrial localization amino acid sequence/base editor fusion protein can be encoded by a nucleic acid and delivered using any appropriate nucleic acid deliver method including, but not limited to, the methods disclosed herein as well as ionophoresis, microspheres (e.g. bioadhesive microspheres), nanoparticles, dendritic polymers, liposomes, hydrogels, cyclodextrins, and proteinaceous vectors.

In embodiments, the mitochondrial localization amino acid sequence is the mitochondrial localization amino acid sequence of cytochrome c oxidase subunit VIII (Coxa) sequence. In embodiments, the mitochondrial localization amino acid sequence is the mitochondrial localization amino acid sequence of superoxide dismutase 2 (SOD2).

In embodiments, the RNA-guided DNA endonuclease enzyme with modified endonuclease activity is dCas9 or nCas9. In embodiments, the RNA-guided DNA endonuclease enzyme with modified endonuclease activity is a “nickase” mutant. In embodiments, the nickase is an SpCas9 nickase. In embodiments, the nickase is SpCas9D10A. In embodiments, the nickase is SpCas9H840A.

In embodiments, the RNA-guided DNA endonuclease enzyme has no nuclear localization sequence.

In another aspect, there is provided a protein including a mitochondrial localization amino acid sequence attached to a base editor fusion protein including a nucleotide base-modifying enzyme and an RNA-guided DNA endonuclease enzyme with modified endonuclease activity. In embodiments, the mitochondrial localization amino acid sequence is covalently attached to a base editor fusion protein including a nucleotide base-modifying enzyme and an RNA-guided DNA endonuclease enzyme with modified endonuclease activity.

In embodiments, the mitochondrial localization amino acid sequence is N-terminal to said RNA-guided DNA endonuclease enzyme.

In embodiments, the mitochondrial localization amino acid sequence is a cytochrome c oxidase subunit VIII (Cox8) sequence.

In embodiments, the mitochondrial localization amino acid sequence is a superoxide dismutase 2 (SOD2) sequence.

In embodiments, the RNA-guided DNA endonuclease enzyme with modified endonuclease activity is dCas9 or nCas9. In embodiments, the RNA-guided DNA endonuclease enzyme with modified endonuclease activity is a “nickase” mutant. In embodiments, the nickase is an SpCas9 nickase. In embodiments, the nickase is SpCas9D10A. In embodiments, the nickase is SpCas9H840A.

In embodiments, the nucleotide base-modifying enzyme is cytidine deaminase or adenosine deaminase.

In embodiments, the RNA-guided DNA endonuclease enzyme has no nuclear localization sequence.

In embodiments, the nucleotide base-modifying enzyme includes a nuclear localization sequence.

In embodiments, any of the proteins above further include a nuclear export signal (NES). In embodiments, the NES is the NS2 protein of Minute Virus of Mice. In embodiments, the NES is the HIV-1 Rev NES.

In another aspect, provided herein are nucleic acids that encode any of the proteins described above. In embodiments, the nucleic acid further includes a 3′UTR sequence that directs mRNA to mitochondria, such as the 3′UTR of SOD2.

While not wishing to be held by theory, Applicants found that in certain cases, such as with cytidine deaminase, a nuclear localization sequence was found within the coding region. As a consequence, compositions that included cytidine deaminase in the fusion sequence were not always sequestered to mitochondria. Applicants surprisingly found that the inclusion of a 3′UTR of SOD2 would correctly direct the compositions to mitochondria.

In embodiments, the protein or its encoding nucleic acid is bound to or otherwise complexed with the delivery vehicle. The binding may be covalent or non-covalent. The term “delivery vehicle” or “carrier” refers to any support structure that brings about the transfer of a component of genetic material or a protein. Genetic material includes but is not limited to DNA, RNA or fragments thereof and proteins or polypeptides comprise amino acids and include but are not limited to antigens, antibodies, ligands, receptors or fragments thereof. Delivery vehicles include but are not limited to vectors such as viruses (examples include but are not limited to retroviruses, adenoviruses, adeno-associated viruses, pseudotyped viruses, replication competent viruses, herpes simplex virus), virus capsids, liposomes or liposomal vesicles, lipoplexes, polyplexes, dendrimers, macrophages, artificial chromosomes, nanoparticles, polymers and also hybrid particles, examples of which include virosomes. Delivery vehicles may have multiple surfaces and compartments for attachment and storage of components. These include but are not limited to outer surfaces and inner compartments.

In embodiments, the delivery vehicle is a nanoparticle or a lipid particle or a viral vector. Any nanoparticles known for protein or nucleic acid delivery can be used for the invention described herein. Nanoparticles are particles between 1 and 100 nanometers in size. Recent dramatic advances in nanotechnology have led to the development of a variety of nanoparticles (NPs) that provide valuable tools. Numerous nanomaterials such as polymers, liposomes, protein based NPs and inorganic NPs have been developed and a variety of particles are currently being evaluated in clinical studies with promising initial results; and some liposomal NPs are approved by the FDA. One of the major advantages of using these NPs is that they offer targeted tissue/site delivery. Their small size allows NPs to escape through blood vessels at the target site through the leaky vascular structure (Enhanced permeability and retention effect). In addition to this passive mechanism, a variety of targeting moieties can be attached to NPs to confer active targeting capability. Exemplary nanoparticles that can be used for delivering compositions described herein include, but are not limited to, solid nanoparticles (e.g., metals such as silver, gold, iron, titanium), non-metal, lipid-based solids (e.g., liposome), polymers (e.g., polyethylenimene, dendrimer), suspensions of nanoparticles, or combinations thereof (e.g., polyethylenimene-liposome, dendrisome). Any compositions described herein (such as Mito-Cas9, mito-Cpf1, or other mito-RNA guided nucleases (mito-RGN)) may be delivered in nanopoarticle complexes in the form of protein, DNA, or mRNA. Additional information about nanoparticles that can be used by the compositions described herein can be found in Coelho et al., N Engl J Med 2013369:819-29, Tabernero et al., Cancer Discovery, April 2013, Vol. 3, No. 4, pages 363-470, Zhang et al., WO2015089419 A2, and Zuris J A et al., Nat Biotechnol. 2015; 33(1):73-80, each of which is incorporated herein by reference.

In embodiments, the vector is a replication-incompetent viral vector. For example, the replication-incompetent viral vector is a replication-incompetent DNA viral vector (including, but is not limited to, adenoviruses, adeno-associated viruses). For example, the replication-incompetent viral vector is a replication-incompetent RNA viral vector (including, but is not limited to, replication defective retroviruses, lentiviruses, and rabies viruses).

In embodiments, the delivery vehicle is a lipid particle-a particle having lipid as a component, such as liposomes or liposomal vesicles or lipoplexes. Liposomes, also known as vesicles, are generally composed of phospholipids and other lipid components such as cholesterol. They can function as carriers whose essential structural feature is a bipolar lipid membrane which envelops an aqueous core volume in which pharmacological agents are solubilized and therefore encapsulated. Various lipid formulations and methods for their preparation have been described for the delivery of pharmaceutically active agents to a host. For example, Geho and Lau in U.S. Pat. No. 4,603,044 describe a targeted liposomal delivery system for delivery of a drug to the hepatobiliary receptors of the liver. The system is composed of a drug or diagnostic agent encapsulated in or associated with lipid membrane structures in the form of vesicles or liposomes, and a molecule having a fatty substituent attached to the vesicle wall and a target substituent which is a biliary attracted chemical, such as a substituted iminodiacetate complex. Several cationic lipid reagents have become commercially available for transfecting eukaryotic cells. These examples include Lipofectin® (DOTMA:DOPE) (Invitrogen, Carlsbad, Calif.), LipofectAmine™ (DOSPA:DOPE)(Invitrogen), LipofectAmine2000™ (Invitrogen), LipofectAmine 3000™ (Invitrogen), Lipofectamine RNAiMax™ (Invitrogen), Lipofectamme LTX™ (Thermo Fisher Scientific), Fugene®, Transfectam® (DOGS), Effectene®, DC-Choi. US Patent Publication No. 20050019923 involves cationic dendrimers for delivering bioactive molecules, such as polynucleotide molecules, peptides and polypeptides and/or pharmaceutical agents, to a mammalian body, given the low toxicity and targeting specificity. Other derivatives of cationic dendrimer mentioned in Bioactive Polymers, US published application 20080267903, may also be suitable delivery vehicles.

Various polymeric formulations of biologically active agents and methods for their preparation have been described. U.S. Pat. Nos. 3,773,919, 3,991,776, 4,076,779, 4,093,709, 4,118,470, 4,131,648, 4,138,344, 4,293,539 and 4,675,189, inter alia, disclose the preparation and use of biocompatible, biodegradable polymers, such as poly (lactic acid), poly(glycolic acid), copolymers of glycolic and lactic acids, poly (o-hydroxycarboxy lie acid), polylactones, polyacetals, polyorthoesters and polyorthocarbonates, for the encapsulation of drugs and medicaments. These polymers mechanically entrap the active constituents and later provide controlled release of the active ingredient via polymer dissolution or degradation. Certain condensation polymers formed from divinyl ethers and polyols are described in Polymer Letters, 18,293 (1980). Polymers have proven to be successful controlled-release drug delivery devices.

More information about liposomal constructs or polymeric constructs that can be used for the present invention can be found at Schwendener R A et al., Ther Adv Vaccines. 2014 November; 2(6): 159-182; Li Y et al., J Gene 2011, Med 13: 60-72; Pichon C et al., Methods Mol Biol 2013 969: 247-274; McNamara M A et al., J Immunol Res. 2015; 2015: 794528; Sayour E. J. et al., Journal for Immunotherapy of Cancer. 2015; 3, article 13; Bettinger T. et al, Current Opinion in Molecular Therapeutics. 2001; 3(2):116-124; Lu D. et al., Cancer Gene Therapy. 1994; 1(4):245-252; Wasungu L. et al., Journal of Controlled Release. 2006; 116(2):255-264; Little S. et al., Proceedings of the National Academy of Sciences of the United States of America. 2004; 101(26):9534-9539; Phua K. et al., Journal of Controlled Release. 2013; 166(3):227-233; Su X et al., Molecular Pharmaceutics. 2011; 8(3):774-787; Phua K. K. L. et al., Nanoscale. 2014; 6(14):7715-7729; Phua K. K. L. et al., Scientific Reports. 2014; 4, article 5128.

In embodiments, the protein or its encoding nucleic acid is encapsulated within said delivery vehicle. Encapsulation can be carried out by any methods known in the art.

In another aspect, provided herein are vectors that include any of the nucleic acids disclosed above. In embodiments, the vector is a replication incompetent viral vector. In embodiments, the replication incompetent viral vector is a replication incompetent lentiviral, adeno-associated viral, or adenoviral vector.

In another aspect, there is provided a pharmaceutical composition including a composition disclosed herein, a protein disclosed herein, a nucleic acid disclosed herein, a vector disclosed herein, and a pharmaceutically acceptable excipient. In embodiments, the pharmaceutical compositions also include a single-guide RNA (sgRNA).

In embodiments the single-guide RNA (sgRNA) sequence can include a mitochondrial localization sequence (MLS). Various embodiments of MLS/sgRNA are disclosed in U.S. patent application Ser. No. 16/307,128, entitled “COMPOSITIONS AND METHODS FOR MITOCHONDRIAL GENOME EDITING”, filed Dec. 4, 2018, and International Patent Application No. PCT/US2019/049165, entitled “CATIONIC COMPOUNDS FOR DELIVERY OF NUCLEIC ACIDS”, filed Aug. 30, 2019, which are incorporated by reference in their entireties.

Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylase or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A pharmaceutical composition of the invention can be administered to a subject in many of the well-known methods currently used for chemotherapeutic treatment. For example, for treatment of cancers, a composition of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

### III. METHODS

In another aspect, there is provided methods of altering expression of at least one mitochondrial nucleic acid sequence, the method including introducing into an eukaryotic cell a composition as disclosed herein, a protein as disclosed herein, a nucleic acid as disclosed herein, or a vector as disclosed herein, and, optionally, a single-guide RNA (sgRNA) as disclosed herein.

In embodiments, the eukaryotic cell is an oocyte. In embodiments, the eukaryotic cell is part of a fertilized embryo. In embodiments, the cell is an in vitro cell.

Levels of expression may be quantitated for absolute comparison, or relative comparisons may be made.

In some embodiments upregulation of expression may be considered to be present when the level of expression in the test sample is at least 1.1 times that of a reference level. More preferably, the level of expression may be selected from one of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4 at least 2.5, at least 2.6, at least 2,7, at least 2.8, at least 2.9, at least 3.0, at least 3.5, at least 4.0, at least 5.0, at least 6.0, at least 7.0, at least 8.0, at least 9.0, or at least 10.0 times that of the reference level.

In some embodiments downregulation of expression may be considered to be present when the level of expression in the test sample is at least 1.1 times lower than that of a reference level. More preferably, the level of expression may be selected from one of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4 at least 2.5, at least 2.6, at least 2.7, at least 2.8, at least 2.9, at least 3.0, at least 3.5, at least 4.0, at least 5.0, at least 6.0, at least 7.0, at least 8.0, at least 9.0, or at least 10.0 times lower than that of the reference level.

Expression levels may be determined by one of a number of known in vitro assay techniques, such as PCR based assays, in situ hybridization assays, flow cytometry assays, immunological or immunohistochemical assays.

In embodiments, the method may further comprise a step of determining expression level (pre-treated level) of at least one mitochondrial nucleic acid sequence before introducing the nucleic acid into the eukaryotic cell. In embodiments, the method may further comprise another step of determining expression level (post-treated level) of the same at least one mitochondrial nucleic acid sequence after introducing the nucleic acid into the eukaryotic cell. In embodiments, the method may further comprise a step of comparing the difference of the pre-treated level and the post-treated level in order to determine if the expression of the at least one mitochondrial nucleic acid sequence of interest has been altered.

In another aspect, there is provided a method of treating a mitochondrial disorder in a subject in need thereof, the method including administering to the subject an effective amount of a pharmaceutical composition disclosed herein.

Further to aspect or embodiments disclosed herein providing a method of treating a mitochondrial disorder in a subject in need thereof, in embodiments the mitochondrial disorder is selected from the group consisting of Myoclonic Epilepsy with Ragged Red Fibers (MERRF); Mitochondrial Myopathy, Encephalopathy, Lactacidosis, and Stroke (MELAS); Maternally Inherited Diabetes and Deafness (MIDD); Leber's Hereditary Optic Neuropathy (LHON); chronic progressive external ophthalmoplegia (CPEO); Leigh Disease; Kearns-Sayre Syndrome (KSS); Friedreich's Ataxia (FRDA); Co-Enzyme Ql0 (CoQl0) Deficiency; Complex I Deficiency; Complex II Deficiency; Complex III Deficiency; Complex IV Deficiency; Complex V Deficiency; other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's Disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; and cancer.

### IV. KITS

In another aspect, there is provided a kit including a composition disclosed herein, a protein described herein, nucleic acid disclosed herein, a vector disclosed herein, or a pharmaceutical composition disclosed herein. The kit may include an sgRNA. The kit may include instructions for use.

### EMBODIMENTS

Embodiment 1. A composition comprising a delivery vehicle and a protein, said protein comprising a mitochondrial localization amino acid sequence covalently attached to a base editor fusion protein comprising a nucleotide base-modifying enzyme and an RNA-guided DNA endonuclease enzyme with modified endonuclease activity, and wherein said protein is bound to said delivery vehicle.

Embodiment 2. The composition of embodiment 1, wherein said mitochondrial localization amino acid sequence is covalently attached to said base editor fusion protein.

Embodiment 3. The composition of embodiment 1 or 2, wherein said mitochondrial localization amino acid sequence is N-terminal to said RNA-guided DNA endonuclease enzyme with modified endonuclease activity.

Embodiment 4. The composition of any of embodiments 1 to 3, wherein said delivery vehicle is a nanoparticle or a lipid particle or a viral vector.

Embodiment 5. The composition of any of embodiments 1 to 4, wherein said protein is encapsulated within said delivery vehicle.

Embodiment 6. The composition of any of embodiments 1 to 5, wherein said mitochondrial localization amino acid sequence is a cytochrome c oxidase subunit VIII (Cox8) sequence.

Embodiment 7. The composition of any of embodiments 1 to 5, wherein said mitochondrial localization amino acid sequence is a superoxide dismutase 2 (SOD2) sequence.

Embodiment 8. The composition of any of embodiments 1 to 7, wherein said RNA-guided DNA endonuclease enzyme with modified endonuclease activity is dCas9 or nCAS9.

Embodiment 9. The composition of any of embodiments 1 to 7, wherein said RNA-guided DNA endonuclease enzyme with modified endonuclease activity is a Cas9 nickase.

Embodiment 10. The composition of embodiment 9, wherein said Cas9 nickase is SpCas9D10A or SpCas9H840A

Embodiment 11. The composition of any of embodiments 1 to 10, wherein said nucleotide base-modifying enzyme is cytidine deaminase or adenosine deaminase.

Embodiment 12. The composition of any of embodiments 1 to 11, wherein said RNA-guided DNA endonuclease enzyme has no nuclear localization sequence.

Embodiment 13. The composition of any of embodiments 1 to 11, wherein said nucleotide base-modifying enzyme comprises a nuclear localization sequence.

Embodiment 14. A composition comprising a delivery vehicle complexes with a nucleic acid, said nucleic acid encoding a mitochondrial localization amino acid sequence covalently attached to a base editor fusion protein comprising a nucleotide base-modifying enzyme and an RNA-guided DNA endonuclease enzyme with modified endonuclease activity.

Embodiment 15. The composition of embodiment 14, wherein said mitochondrial localization amino acid sequence is N-terminal to said RNA-guided DNA endonuclease enzyme with modified endonuclease activity.

Embodiment 16. The composition of embodiment 14 or 15, wherein said delivery vehicle is a nanoparticle or a lipid particle or a viral vector.

Embodiment 17. The composition of any of embodiments 14 to 16, wherein said nucleic acid is encapsulated within said delivery vehicle.

Embodiment 18. The composition of any of embodiments 14 to 17, wherein said mitochondrial localization amino acid sequence is a cytochrome c oxidase subunit VIII (Cox8) sequence.

Embodiment 19. The composition of any of embodiments 14 to 17, wherein said mitochondrial localization amino acid sequence is a superoxide dismutase 2 (SOD2) sequence.

Embodiment 20. The composition of any of embodiments 14 to 19, wherein said RNA-guided DNA endonuclease enzyme with modified endonuclease activity is dCas9 or nCAS9.

Embodiment 21. The composition of any of embodiments 14 to 19, wherein said RNA-guided DNA endonuclease enzyme with modified endonuclease activity is a Cas9 nickase.

Embodiment 22. The composition of embodiment 21, wherein said Cas9 nickase is SpCas9D10A or SpCas9H840A

Embodiment 23. The composition of any of embodiments 14 to 22, wherein said nucleotide base-modifying enzyme is cytidine deaminase or adenosine deaminase.

Embodiment 24. The composition of any of embodiments 14 to 23, wherein said RNA-guided DNA endonuclease enzyme has no nuclear localization sequence.

Embodiment 25. The composition of any of embodiments 14 to 23, wherein said nucleotide base-modifying enzyme comprises a nuclear localization sequence.

Embodiment 26. A protein comprising a mitochondrial localization amino acid sequence covalently attached to a base editor fusion protein, said base editor fusion protein comprising a nucleotide base-modifying enzyme and an RNA-guided DNA endonuclease enzyme with modified endonuclease activity.

Embodiment 27. The protein of embodiment 26, wherein said mitochondrial localization amino acid sequence is N-terminal to said RNA-guided DNA endonuclease enzyme.

Embodiment 28. The protein of embodiment 26 or 27, wherein said mitochondrial localization amino acid sequence is a cytochrome c oxidase subunit VIII (Cox8) sequence.

Embodiment 29. The protein of embodiment 26 or 27, wherein said mitochondrial localization amino acid sequence is a superoxide dismutase 2 (SOD2) sequence.

Embodiment 30. The protein of any of embodiments 26 to 29, wherein said RNA-guided DNA endonuclease enzyme is dCas9 or nCAS9.

Embodiment 31. The protein of any of embodiments 26 to 29, wherein said RNA-guided DNA endonuclease enzyme with modified endonuclease activity is a Cas9 nickase.

Embodiment 32. The protein of embodiment 31, wherein said Cas9 nickase is SpCas9D10A or SpCas9H840A.

Embodiment 33. The protein of any of embodiments 26 to 32, wherein said nucleotide base-modifying enzyme is cytidine deaminase or adenosine deaminase.

Embodiment 34. The protein of any of embodiments 26 to 33, wherein said RNA-guided DNA endonuclease enzyme has no nuclear localization sequence.

Embodiment 35. The protein of any of embodiments 26 to 33, wherein said nucleotide base-modifying enzyme comprises a nuclear localization sequence.

Embodiment 36. A nucleic acid encoding said protein of any of embodiments 26-35.

Embodiment 37. The nucleic acid of embodiment 36, further comprising a 3′UTR of SOD2.

Embodiment 38. A vector comprising a nucleic acid of embodiment 36 or 37.

Embodiment 39. The vector of any of embodiments 36 to 38, wherein said vector is a replication incompetent viral vector.

Embodiment 40. The vector of embodiment 39, wherein said replication incompetent viral vector is a replication incompetent lentiviral, adeno-associated viral, or adenoviral vector.

Embodiment 41. A pharmaceutical composition comprising the composition of any one of embodiments 1 to 25, the protein of any one of claims 26 to 35, the nucleic acid of embodiment 36 or 37, or a vector of any one of claims 38 to 40, and a pharmaceutically acceptable excipient.

Embodiment 42. The pharmaceutical composition of embodiment 41, further comprising a single-guide RNA (sgRNA).

Embodiment 43. A method of altering expression of at least one mitochondrial nucleic acid sequence, the method comprising introducing into an eukaryotic cell the composition of any one of embodiments 1 to 25, the protein of any one of claims 26 to 35, the nucleic acid of embodiment 36 or 37, or a vector of any one of claims 38 to 40, and a single-guide RNA (sgRNA).

Embodiment 44. The method of embodiment 43, wherein said eukaryotic cell is an oocyte.

Embodiment 45. The method of embodiment 43, wherein said eukaryotic cell is part of a fertilized embryo.

Embodiment 46. The method of any of embodiments 43 to 45, wherein said mitochondrial nucleic acid sequence comprises at least one mutation or deletion.

Embodiment 47. A method of treating a mitochondrial disorder in a subject in need thereof, the method comprising administering to said subject an effective amount of the composition of any one of embodiments 1 to 25, the protein of any one of claims 26 to 35, the nucleic acid of embodiment 36 or 37, or a vector of any one of claims 38 to 40, or a pharmaceutical composition of embodiment 41, and single-guide RNA (sgRNA).

Embodiment 48. The method of embodiment 47, wherein said mitochondrial disorder is selected from the group consisting of Myoclonic Epilepsy with Ragged Red Fibers (MERRF); Mitochondrial Myopathy, Encephalopathy, Lactacidosis, and Stroke (MELAS); Maternally Inherited Diabetes and Deafness (MIDD); Leber's Hereditary Optic Neuropathy (LHON); chronic progressive external ophthalmoplegia (CPEO); Leigh Disease; Kearns-Sayre Syndrome (KSS); Friedreich's Ataxia (FRDA); Co-Enzyme Q10 (CoQ10) Deficiency; Complex I Deficiency; Complex II Deficiency; Complex III Deficiency; Complex IV Deficiency; Complex V Deficiency; other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's Disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; and cancer.

Embodiment 49. A kit, comprising a composition of any one of embodiments 1 to 25, the protein of any one of claims 26 to 35, the nucleic acid of embodiment 36 or 37, or a vector of any one of claims 38 to 40, or a pharmaceutical composition of embodiment 41.

Embodiment 50. The kit of embodiment 49, further comprising a single-guide RNA (sgRNA).

Embodiment 51. A method of altering expression of at least one mitochondrial nucleic acid sequence, the method comprising introducing into an eukaryotic cell the protein of any of embodiments 26 to 35, the nucleic acid of embodiments 36 or 37, the vector of any of embodiment 38 to 40, or the pharmaceutical composition of embodiment 41,

and a single-guide RNA (sgRNA).

Embodiment 52. The method of embodiment 51, wherein said eukaryotic cell is an oocyte.

Embodiment 53. The method of embodiment 51, wherein said eukaryotic cell is part of a fertilized embryo.

Embodiment 53. The method of any of embodiments 51 to 53, wherein said mitochondrial nucleic acid sequence comprises at least one mutation or deletion.

### P EMBODIMENTS

P Embodiment 1. A composition comprising a delivery vehicle and a protein, said protein comprising a mitochondrial localization amino acid sequence covalently attached to a base editor fusion protein comprising a nucleotide base-modifying enzyme and an RNA-guided DNA endonuclease enzyme with modified endonuclease activity, and wherein said protein is bound to said delivery vehicle.

P Embodiment 2. The composition of embodiment 1, wherein said delivery vehicle is a nanoparticle or a lipid particle or a viral vector.

P Embodiment 3. The composition of embodiment 1, wherein said protein is encapsulated within said delivery vehicle.

P Embodiment 4. The composition of embodiment 1, wherein said mitochondrial localization amino acid sequence is N-terminal to said RNA-guided DNA endonuclease enzyme with modified endonuclease activity.

P Embodiment 5. The composition of embodiment 1, wherein said mitochondrial localization amino acid sequence is a cytochrome c oxidase subunit VIII (Cox8) sequence.

P Embodiment 6. The composition of embodiment 1, wherein said mitochondrial localization amino acid sequence is a superoxide dismutase 2 (SOD2) sequence.

P Embodiment 7. The composition of embodiment 1, wherein said RNA-guided DNA endonuclease enzyme with modified endonuclease activity is dCas9 or nCAS9.

P Embodiment 8. The composition of embodiment 1, wherein said RNA-guided DNA endonuclease enzyme with modified endonuclease activity is a Cas9 nickase.

P Embodiment 9. The composition of embodiment 8, wherein said Cas9 nickase is SpCas9D10A or SpCas9H840A.

P Embodiment 10. The composition of embodiment 1, wherein said nucleotide base-modifying enzyme is cytidine deaminase or adenosine deaminase.

P Embodiment 11. The composition of embodiment 1, wherein said RNA-guided DNA endonuclease enzyme has no nuclear localization sequence.

P Embodiment 12. The composition of embodiment 1, wherein said nucleotide base-modifying enzyme comprises a nuclear localization sequence.

P Embodiment 13. A composition comprising a delivery vehicle and a nucleic acid encoding said protein of one of embodiments 1 to 12.

P Embodiment 14. A protein comprising a mitochondrial localization amino acid sequence covalently attached to a base editor fusion protein comprising a nucleotide base-modifying enzyme and an RNA-guided DNA endonuclease enzyme with modified endonuclease activity.

P Embodiment 15. The protein of embodiment 14, wherein said mitochondrial localization amino acid sequence is N-terminal to said RNA-guided DNA endonuclease enzyme.

P Embodiment 16. The protein of embodiment 14, wherein said mitochondrial localization amino acid sequence is a cytochrome c oxidase subunit VIII (Cox8) sequence.

P Embodiment 17. The protein of embodiment 14, wherein said mitochondrial localization amino acid sequence is a superoxide dismutase 2 (SOD2) sequence.

P Embodiment 18. The protein of embodiment 14, wherein said RNA-guided DNA endonuclease enzyme is dCas9 or nCAS9.

P Embodiment 19. The composition of embodiment 14, wherein said RNA-guided DNA endonuclease enzyme with modified endonuclease activity is a Cas9 nickase.

P Embodiment 20. The composition of embodiment 19, wherein said Cas9 nickase is SpCas9D10A or SpCas9H840A

P Embodiment 21. The protein of embodiment 14, wherein said nucleotide base-modifying enzyme is cytidine deaminase or adenosine deaminase.

P Embodiment 22. The protein of embodiment 14, wherein said RNA-guided DNA endonuclease enzyme has no nuclear localization sequence.

P Embodiment 23. The protein of embodiment 14, wherein said nucleotide base-modifying enzyme comprises a nuclear localization sequence.

P Embodiment 24. A nucleic acid encoding said protein of any of embodiments 14 to 23.

P Embodiment 25. The nucleic acid of embodiment 24, further comprising a 3′UTR of SOD2.

P Embodiment 26. A vector comprising a nucleic acid of any one of the embodiments 24

P Embodiment 27. The vector of embodiment 26, wherein said vector is a replication incompetent viral vector.

P Embodiment 28. The vector of embodiment 27, wherein said replication incompetent viral vector is a replication incompetent lentiviral, adeno-associated viral, or adenoviral vector.

P Embodiment 29. A pharmaceutical composition comprising the composition of one of claims 1 to 13, the protein of one of claims 14 to 19, the nucleic acid of one of claims 24 to 25, or a vector of one of claims 26 to 28, and a pharmaceutically acceptable excipient.

P Embodiment 30. The pharmaceutical composition of embodiment 29, further comprising a single-guide RNA (sgRNA).

P Embodiment 31. A method of altering expression of at least one mitochondrial nucleic acid sequence, the method comprising introducing into an eukaryotic cell the composition of one of embodiments 1 to 13, the protein of one of embodiments 14 to 19, the nucleic acid of one of embodiments 24 to 25, or a vector of one of embodiments 26 to 28, and a single-guide RNA (sgRNA).

P Embodiment 32. The method of embodiment 31, wherein said eukaryotic cell is an oocyte.

P Embodiment 33. The method of embodiment 31, wherein said eukaryotic cell is part of a fertilized embryo.

P Embodiment 34. The method of embodiment 31, wherein said mitochondrial nucleic acid sequence comprises at least one mutation or deletion.

P Embodiment 35. A method of treating a mitochondrial disorder in a subject in need thereof, the method comprising administering to said subject an effective amount of the composition of one of embodiments 1 to 13, the protein of one of embodiments 14 to 19, the nucleic acid of one of embodiments 24 to 25, a vector of one of embodiments 26 to 28, or a pharmaceutical composition of embodiment 29 or 30, and single-guide RNA (sgRNA).

P Embodiment 36. The method of embodiment 35, wherein said mitochondrial disorder is selected from the group consisting of Myoclonic Epilepsy with Ragged Red Fibers (MERRF); Mitochondrial Myopathy, Encephalopathy, Lactacidosis, and Stroke (MELAS); Maternally Inherited Diabetes and Deafness (MIDD); Leber's Hereditary Optic Neuropathy (LHON); chronic progressive external ophthalmoplegia (CPEO); Leigh Disease; Kearns-Sayre Syndrome (KSS); Friedreich's Ataxia (FRDA); Co-Enzyme Ql0 (CoQl0) Deficiency; Complex I Deficiency; Complex II Deficiency; Complex III Deficiency; Complex IV Deficiency; Complex V Deficiency; other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; hearing and balance impairments; or other neurological disorders; epilepsy; genetic diseases; Huntington's Disease; mood disorders; nucleoside reverse transcriptase inhibitors (NRTI) treatment; HIV-associated neuropathy; schizophrenia; bipolar disorder; age-associated diseases; cerebral vascular diseases; macular degeneration; diabetes; and cancer.

P Embodiment 37. A kit, comprising a composition of one of embodiments 1 to 13, the protein of one of embodiments 14 to 19, the nucleic acid of one of embodiments 24 to 25, a vector of one of embodiments 26 to 28, or a pharmaceutical composition of embodiment 29.

P Embodiment 38. The kit of embodiment 37, further comprising a single-guide RNA (sgRNA).

### V. EXAMPLES

We have created a base editing technology that localizes to the mitochondrial matrix and targets mitochondrial DNA for site-specific base conversion. This system can be used in therapeutic applications for correcting genetic mutations or defects in mitochondrial DNA (mtDNA) as well as in research applications by introducing genetic variations in the mtDNA of cells and research animals for creating new disease models. The base editor technology incorporates an RNA-guided DNA endonuclease enzyme with modified endonuclease activity such as a catalytically dead base editor (e.g. dCas9 or nCas9) with a nucleotide base-modifying enzyme (e.g. cytidine deaminase or adenosine deaminase protein) that can alter DNA bases without inducing a DNA break. The mtDNA base editor is able to achieve introduction or correction of a point mutation at a target locus rather than stochastic disruption of the entire gene by non-homologous end joining (NHEJ). Embodiments disclosed herein provides base editing technology that specifically targets mtDNA.

The CRISPR base editor plasmid BE4 (Addgene Plasmid #100802) (Komor, A. C., Zhao, K. T., Packer, M. S., Gaudelli, N. M., Waterbury, A. L., Koblan, L. W., Kim, Y. B., Badran, A. H., and Liu, D. R. (2017). Improved base excision repair inhibition and bacteriophage Mu Gam protein yields C:G-to-T:A base editors with higher efficiency and product purity. Sci Adv 3, eaao4774) was modified for mitochondrial localization. Then, we used two distinct strategies to add mitochondrial localized signals. One is by adding SOD2 mitochondrial targeting sequence (MTS) to the 5′ of the BE4 and SOD2 3′UTR to the 3′ of BE4 vector. The other strategy by adding Cox8 MTS to the 5′ of BE4. For both designs, the MTS serves as a peptide signal to direct the MTS-APOBEC1-Cas9n(D10A)-2×UGI protein through the outer and inner mitochondrial membranes. However, in the Cox8 approach, we occasionally observed a localization of the MTS-APOBEC1-Cas9n(D10A)-2×UGI protein in both mitochondria and nucleus. This is because the APOBEC1 protein, which serves as the cytidine deaminase enzymatic function, contains an NLS that cannot easily be removed without potentially disrupting the enzymatic function APOBEC1. Thus, the SOD2 approach also includes a 3′UTR sequence that directs the mRNA to the outer mitochondrial membrane, where it is translated and then immediately imported as a nascent protein into the mitochondria. This approach led to mitochondrial localization of MTS-APOBEC1-Cas9n(D10A)-2×UGI without any discernable nuclear localization.

### Example 1

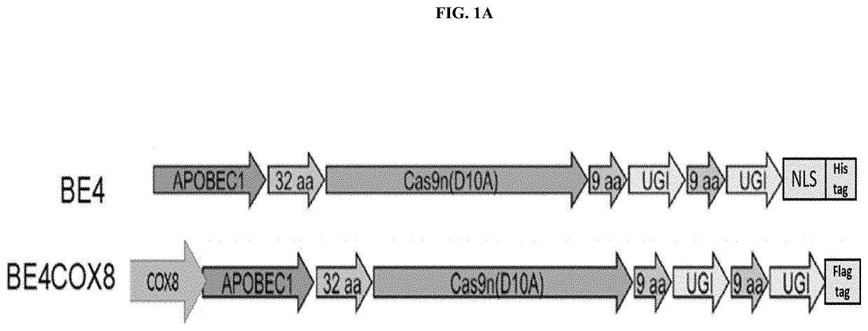

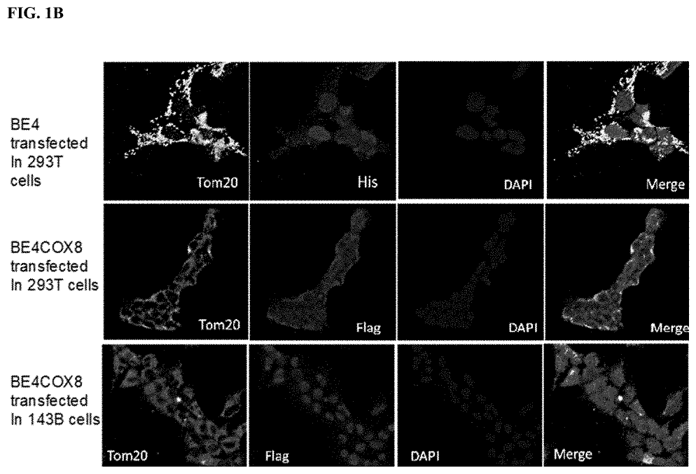

We created a mitochondrial CRISPR BE4COX8 by removing the nuclear localization signals (NLS) from the C-terminus of BE4 and adding mitochondrial targeting sequence (MTS) COX8 peptide at the N-terminus (FIG. 1A). The BE4 base editor is a fusion of the human APOBEC1 cytidine deaminase protein to the N-terminus of the Cas9 D10A nickase with two uracil glycosylase inhibitors (UGI) to the C-terminus, a nuclear localization signal (NLS) and a polyhistidine (His) tag to the C-terminus (Komor et al., 2017). As shown in FIG. 1B, HEK 293T cells transfected by BE4 showed strong nuclear localization of the construct (FIG. 1B, top row); HEK 293T cells transfected by BE4COX8 start to show co-localization of mito-base editor protein and mitochondrial Tom20 protein (FIG. 1B, middle row). However, 143B cells transfected by BE4COX8 still showed nuclear localization of base editor protein, suggesting that the mitochondrial localization of the BE4COX8 construct was not consistent across different cell lines or cell types. The BE4 and BE4COX8 proteins were stained with anti-Flag antibody. Mitochondria are indicated by the staining of Tom20 protein. DAPI indicates the nucleus. The overlap of anti-Flag and anti-Tom2 signals indicated mitochondrial localization, whereas overlap of anti-Flag and DAPI signals indicated nuclear localization.

### Example 2

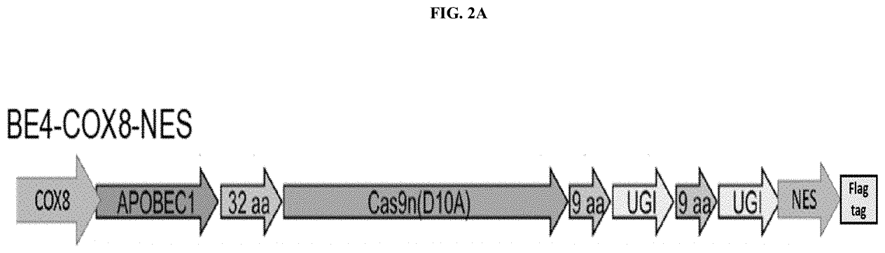

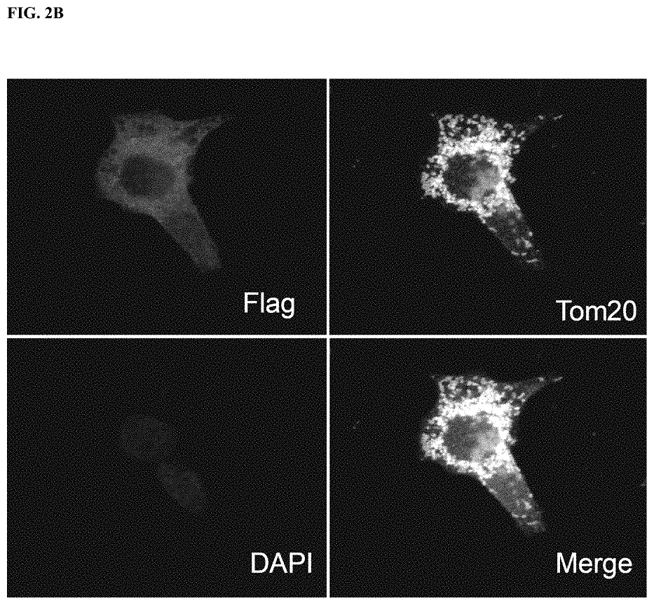

Because the BE4COX8 construct was retained in the nucleus in 143B cells, we hypothesized that the addition of a nuclear export signal (NES) would improve mitochondrial localization. We created a mitochondrial CRISPR BE4-COX8-NES (FIG. 2A) by adding a C-terminal NES (derived from the HIV-1 Rev protein) to the BE4COX8 construct from FIG. 1A. This eliminated nuclear localization and dramatically improved mitochondrial localization in 143B cells (FIG. 2B).

### Example 3

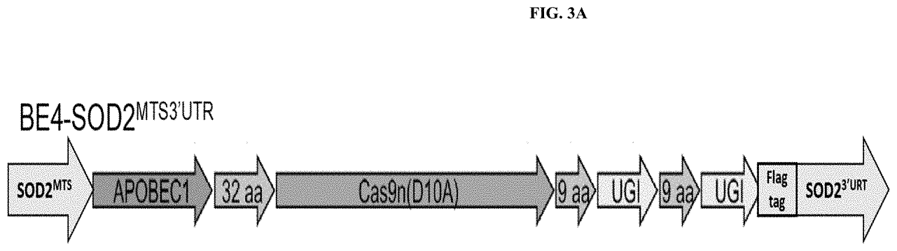

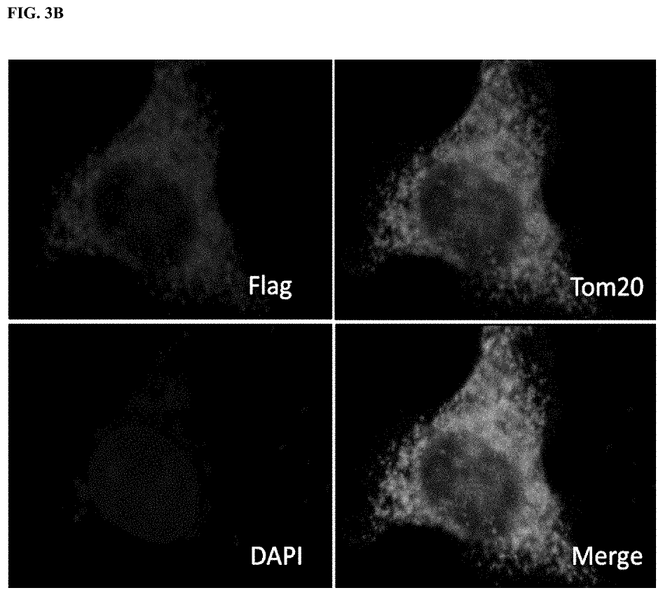

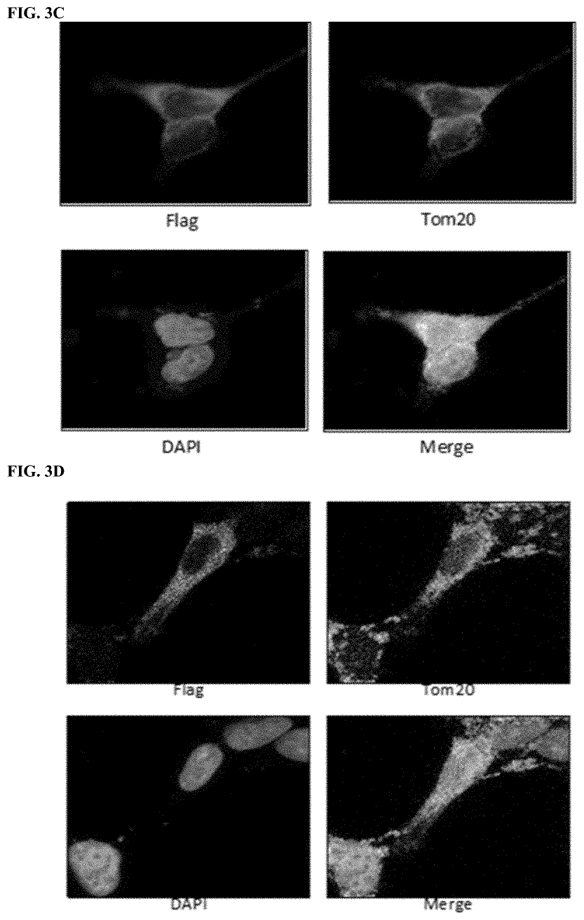

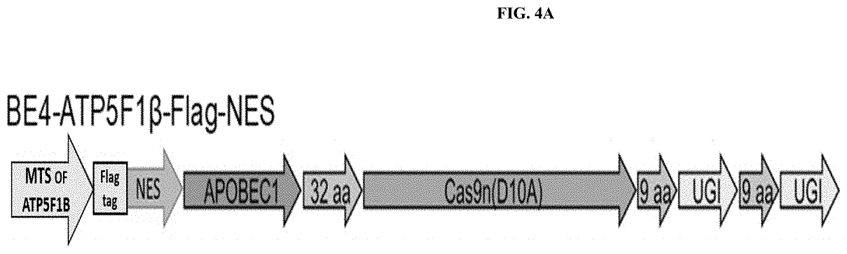

We created a mitochondrial CRISPR BE4-SOD2MTS3′UTR by removing the nuclear localization signals (NLS) from the C-terminus of the BE4 and adding the SOD2 mitochondrial targeting sequence (MTS) peptide at the N-terminus and the 3′UTR of the SOD2 gene to the 3′-end of the transcript, immediately after the STOP codon of the FLAG tag (FIG. 3A). The combination of the SOD2 MTS and SOD2 3′-UTR appeared necessary for the natural mitochondrial localization of the human SOD2 protein (FIGS. 3B-3D).

### Example 4

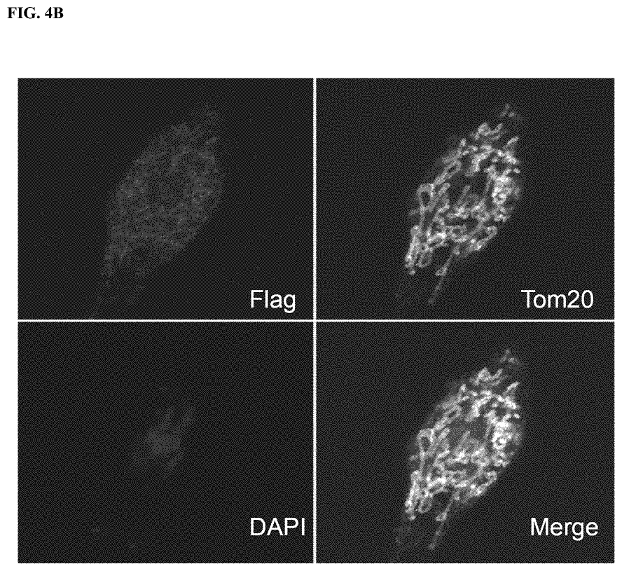

We created a mitochondrial CRISPR BE4-ATP5F1β-Flag-NES by removing the nuclear localization signals (NLS) from the C-terminus of the BE4 and adding the ATP5F113 mitochondrial targeting sequence (MTS) peptide at the N-terminus and the nuclear export sequence (NES) from the NS2 protein of Minute Virus of Mice immediately before the APOBEC1 gene (FIG. 4A). Co-localization of mito-base editor protein and mitochondrial Tom20 protein was observed (FIG. 4B).

### Example 5

METHODS: BE4 or mitoBE4 plasmid DNAs were transfected by using Lipofectmine 3000 in HEK 293T cells or 143B cells. After culturing for 3 days, cells were fixed and Immunofluorescence were performed. Mitochondrial protein Tom20 was blotted by Tom20 Antibody (FL-145) (Santa Cruz, sc-11415), nuclear APOBEC-nCas9 is blotted by anti-6×His tag antibody [HIS.H8] (Abcam, ab18184), and mito-APOBEC-nCas9 fusion protein was blotted by Flag antibody DYKDDDDK Tag (9A3), (Cell signaling technology, #8146) or Sino Biological Anti-DYKDDDDK (FLAG® epitope Tag) Antibody.

### VI. INFORMAL SEQUENCE LISTING

Cox8 amino acid sequence:
(SEQ ID NO: 1)
MSVLTPLLLRGLTGSARRLPVPRAKIHSL
Mitochondrial localization sequence derived
from Cox8:
(SEQ ID NO: 2)
ATGTCCGTCCTGACGCCGCTGCTGCTGCGGGGCTTGACAGGCTCGGCCC
GGCGGCTCCCAGTGCCGCGCGCCAAGATCCATTCGTTG
Cox8 nucleotide sequence (GenBank J04823.1):
(SEQ ID NO: 3)
GGCTACGGCTGACCGTTTTTTGTGGTGTACTCCGTGCCATCATGTCCGT
CCTGACGCCGCTGCTGCTGCGGGGCTTGACAGGCTCGGCCCGGCGGCTC
CCAGTGCCGCGCGCCAAGATCCATTCGTTGCCGCCGGAGGGGAAGCTTG
GGATCATGGAATTGGCCGTTGGGCTTACCTCCTGCTTCGTGACCTTCCT
CCTGCCAGCGGGCTGGATCCTGTCACACCTGGAGACCTACAGGAGGCCA
GAGTGAAGGGGTCCGTTCTGTCCCTCACACTGTGACCTGACCAGCCCCA
CCGGCCCATCCTGGTCATGTTACTGCATTTGTGGCCGGCCTCCCCTGGA
TCATGTCATTCAATTCCAGTCACCTCTTCTGCAATCATGACCTCTTGAT
GTCTCCATGGTGACCTCCTTGGGGGTCACTGACCCTGCTTGGTGGGGTC
CCCCTTGTAACAATAAATCTATTTAAACTTT
SOD2 nucleotide sequence (Genbank NG_008729):
(SEQ ID NO: 4)
TCAAGTGTTTTTTTTGTTTTGTTTTTTGTTTGTTTGTTTGTTTGTTTTT
GACACTGAGTCCCACTCTGTCGCCCAGGCTGGAGTGCAGTGGCGAGATC
TCAGCTCACTGCAGCTGCCGCCTCCCGGGTTCAAGCGATTCTTCTGCCT
CAGCCTCCCAAGTAGCTGGGACTACAGGTGCGTGCCACCACGCCTAGCT
AATTTTTGTATTTTTAGTAAAGACAGAGTTTCACCATATTGGCCAGGCT
GGTCTCCAATTCCTGACCTCATGATCCACCTGCCTCAGCCTCCCAAAGT
CCTGGGATTATAGGCGTGAGCCACTGGGCCCTGCCAATTTTTGTATTTT
TAGTAGAGATGGGGTTTCACCATGTTGGCCAGGCTGATCTCGATTTCCC
GACCTCAGATGATCCGCCCACCTCGGCCTCCCAAAGTGTTGGGATTACA
GGCGTGAGCCATGGTGCTGGGCATATACGCAGATTTCTGACTGTGAAGG
GACCAGCACCCCTGACACTGCATCATTCAGGGGTCAACTGTACATTCAT
ATCCCCCTTACTACCTACTAGCTCTATGTGTGACCTTGGTCAAGTCACT
GTCTTTGCTTACAGCAGGAATAAAGCATTTGACTTAGTAGCTAGCAACA
TGGAGGATCTGCTCAATGATGCCGACTCCCTTTGCCCTTTATGGACGTA
ATCAACTCATGATTACCTGCGACACATGCCCACTTTATCCCTTTGTTTA
ACAGAACACTTTTGTTCTGAACCAAGCTCCTCCTTTCCTTTCCAGTAGT
TAATGTGTTCTTTGAGGATGCAATCTAATCGTAATTTTGCCCGAAGTGG
GGAAAAAAATTAAGCTCACATTGAAACCTTAATATAGCTCTATAATCAT
TTTCTCTTTTAGCCTAGATCACTTCATTTTGGCAATTTAAGTACTACTT
TTGGTCCTATTAACAATACCACTCTTGCAAAGGACAGGTTACAACCAAT
TCTCAGATTCCTGCCTGGATAAGATTTGTGACCCCAAATTTTAAAAAAG
CAGAAAAAAACCCCACAAACCACAGGCAGGAAGAAAGAAAAACAAGAGA
ATGTAGCTAAGGTAATTCCATTAATGATCAGTTTATGTCAAAGCATGTG
GAAATAATGCCTTATAGGAGGATATTTAGCAACAACGATTTTTTTAAAC
TCATTTTCTCCTGTAGGTACAGAATTGCAAGTTTTTTCTGACTTCAAAT
GTGTATAGCTGACTGCGTGTGTGTGTGTGTGTGTGTGTGTGTGTGTGTG
TGTGTGTGTGTGTCAGGGTCTCCTTCTCTTGCCCAGACTGGAGTAAGGT
GACATAATCATAGCTCATTGCAGCCATGAACTCTTGGGCTCAAGTGATC
CTCCTGCCTTCAGCCTCCTGGCGTAGCTAGGACTGCAGGCACATGCTAC
CACACCCAGCTACTGTTTTTTGGTAGAGACAGAGTCTTGCTATGTTGCC
TAAGCTGGTCTCAAACTCCTGGCCTCAAGCAATCTTCCCACCTCAGCCT
CTCAAAGAGCTGGGATTACAAGCATGAACCACTGCACCTGGCCCCATAC
CTGACTACATTTCTCAGCTTCCTTTGTGGCTAGGCTGTGCTAGGTAGCC
TTGTACCTAAATTTTGACCAGTGAGCAGCAAGTGGAATTTGTTGGATGA
ATCTACCACAAAGGATCCTTACAATGGAGATAGTGGGGCCAGGCATGGT
GGCTCATGCCTGTAATCCCAGCACTTTGGGAGGCTGAGGCAGGCAGATC
ACTTCAGTCTAGGAGTTCGAGACCAGCCTGGCCAACATGGTGAAACCCC
ATCTCCACTAAAAATACAAAAATTAGCCAGGCATGGTGGCACGCACCTG
TAATCCCAGCTACTCAGGAGGCTAAGGCAGGAGAATCACTTGAACTCAG
GAGGTGGAGGTTGCAGTGAGCCGAGATCGCACCACTGCACTCCAGCCCA
GCAACAAAGCAAGACTCTTGATTCGGAAAGAAAAAATAAAAAAAGGTTG
GGGGAGACAGTGGGAGCCCAGACTTTTGTCCTTCCCCTTGCCTAGAAGG
GAGATGAGGTTGCTGGTGCTGTGGAAGCTATTATGGACCATGAGGCAGC
TTTGAAGACAGAAAGCCTGCATCCTTGATGACTCAATGGAATTTCATCC
CAACCCAGGACTGACTGCCCCAGATCTTTGTTACATGAGGGAATACACT
CTTCTGTGTTTAAGAAACTGTGGTTGGATCTGTTACTGGAATATGAATG
CAGTTAATAACTGGCTCAAAATGACTTCATCTCAACCCACATTGGTTGC
TTCTTCCAAACAATATATAGGTCTGTACTGTGGTGGGTCTCAGGATGGC
TGTGATGTAGCCTTAGGAAGTTTATCTATGGGAAATCCATATTCATGGT
GTCCTGATGTTGCAGAGGACATCCTGAGCTGGCTGGAGTAACTTGGGAC
ACAGGTCAATCGACTGTAATCTAACTTCTGAGGCCATTCAGTACCCTCT
ACAGTGGCCACCTAAAAAAAAGGCAGCCAGGTGTGGTGGCTCAAGCCTA
TATAGATCCCAGAACTTTGGAAGGCTGAGGTGGGAGGATCACTTGAGCC
AAGGAGTTTGAGGCCAGCCTGGGCAACATGAAGAAACTCTGTTTCTACA
AAAAATAAAAAAAATTAGCCAGGCATGGTGGTATGCACCTGTAGTTCCA
GCTTCTTGGGAGGCTGAGGTGGAAGAATGACATGAGCCCAGCAAGTCGC
TGCAATCAGCCGTGATCACGCCGCTGCACTCCAGCCTGGGCGACAAAAA
GAAAAAGAAAACGGAGCCTGTTCACTGGGTGTGGTAGACAAGGTAAACT
TTTCTTTACCTCCCATATCCCACAACCTTGGATGTGCTCACAGTCATGG
TAGTGTTTTGTAATGATGTAGCTGATGACAGGTGTGATGTTGGAGATTC
TTCTACCTGACTGCTGCTATCAGTCCTACCAGCCCCCAACGTTTGGTGC
TTGTTCTAAAGGGCATGTCCTAGGAGTCGCTTTAAACTCTCAAAGTATC
ACTCTCTATACAAACAAGAAGTGCAAGTAAGTAGCCTGAGCTCAGCCTC
CCAATAGGAATATTTCATTATCACTAGATCAAGTCTTTCCATTACAATG
ACTGATCTGTCTCTGAATCCTGTGGATTCATCCTTCAAAATGCCCTTTT
CTTCCAGTTCTCATAGCTAGTGCCCTAAAAGTGACCTGCAGTACCTCCT
GCTGAGACGAATGTACCAGCTTCCTAACTAGCCTGCACTCCCTTCATCC
CCCCAAGTCAGTGCCAGACCACCTTGCCTGAAAAACCACTTTCAGTGTG
TCTCACCTCAGCAGAAATGTTTCTCAGCTTCCAATTAACAATCACATCA
AACCCCTGCTCTTGTCTGCGTTTTAAGGGTATCTATAGGCCGGGCGCCG
TGGCTCCTACCTGTAATCCCAGCACTTTGGAAGGCCGAGGCGGGCAGAT
CACTTGAGGTCAGGCGTTCGAGACCATCCTGACCAACATAGTGAAACCC
CGTCTCTACCAAAAATACAAAAAAAAAAAAAATAGTGGGGCGTGGAGGT
GCACGCCTGTAATTCCAGCTACTCGGGAGGCTGAGGCAGGAGAATCGCT
TGAACCCGGGAGGCAGAGGTTCCAGTGAGCCGACATCGCGACACAGTAC
TCCAGCCTGAGCGACAGAGCGAGGCTGTGTCTCAAAAATAAATAAATAA
TAAATTAAAAAAATAAGAGTATCTATAACCTGGTCCCAGCCTGAATTTC
CTTTTTCACCCCAACACGTAGCCCTAGTTACATTCTTCTGACGTCTGTA
AACAAGCCCAGCCCTTCCTGTTGTGAAGCCAAGTTCAGGTGGTTCCTCT
TCGCCTGACTGTTTTCCCATTCCACTTACCGGAAGCCTAGTCATCCTTC
GGAGGGCTGTACGGGGGTTGCAAGAAGCAACGGAAACGGTTCAGCACCT
GCTACCTTCCATCATATTCTTTTCAATAAAGGGGCAACTCCCGCCAATG
GCAGTGTAGATTTCCTAACCTCTACACATGGAAGATTCACACCATTCAG
GATTGTTGTTTAACTGTTGAGAGAGCACTTGATACTTAACAGCTTACTA
GGCTACAAGACAGCGCAGGAAAGAATCCTCTGTTGTCCTTTTATGTTAT
CCTGAACAGTTGGTTCACAGAGTTACTGTAAACACACAAAACATGACTG
CCAGGGCTTAGTAGTGAGGAAGGTGGGAACTAGTCCTGACTCAGTTAAC
TGTGCCCAGGAGAAGCTGCTTAACCTCAAAGGATTTCACTATTACTAGA
ATCAATAATACCAACCCTAGGGGTAAAAATAAAGATAAATGTGTGCAAA
TCCTGCCTGCAGTCTCGGGCACGTCGTGGGTGTCCAAGAACTGTTCTTA
GGCAGCCGGTGGGGACAAAGTCTGTGTGCCTCCTGTCCTGGAATAGGTC
CCAAGGTCGGCTTACTTGCAAAGCAAGGGTACGGCGCAAGAGTACTGAA
TACGGGTTGGAAGGGCGCTGGCTCTACCCTCAGCTCATAGGCCGGCTGG
GCGGCGCTGACCAGCAGCTAGGCCCCGTCTTCCCTAGGAACGGCCACGG
GGGCCCTGGGAGGGTATGAATGTCTTTTTGCAGTGAGGCCTCTGGACCC
CGCGGCCCCCCGGCAGCGCAACCAAAACTCAGGGGCAGGCGCCGCAGCC
GCCTAGTGCAGCCAGATCCCCGCCGGCACCCTCAGGGGCGGAGCCGGAG
GCAGGGCCTTCGGGCCGTACCAACTCCACGGGGGCAGGGGCCGCCTCCC
TTCGGCCGCGCGCCACTCAAGTACGGCAGACAGGCAGCGAGGTTGCCGA
GGCCGAGGCTAGCCTGCAGCCTCCTTTCTCCCGTGCCCTGGGCGCGGGG
TGTACGGCAAGCGCGGGCGGGCGGGACAGGCACGCAGGGCACCCCCGGG
GTTGGGCGCGGCGGGCGCGGGGCGGGGCCCGCGGGGGGGGGGGGCGGGG
CGGCGGTGCCCTTGCGGCGCAGCTGGGGTCGCGGCCCTGCTCCCCGCGC
TTTCTTAAGGCCCGCGGGCGGCGCAGGAGCGGCACTCGTGGCTGTGGTG
GCTTCGGCAGCGGCTTCAGCAGATCGGCGGCATCAGCGGTAGCACCAGC
ACTAGCAGCATGTTGAGCCGGGCAGTGTGCGGGTGAGAAGAAAGGGGAC
CCGGTCACGGCCCCAAGGGCGAAGGGGCTCGCGGCGGGCAGGGCCTCCG
CGGCAATGGCGACAGTGGCCGCACCGGGCCTGGCGGGACCGGGGCACCT
GCAGGCGGTTCTCCCGGGAGTGCCCGGCGCGGCGGCTGGAGCGGGGATC
CGCAGGGAGGGGACGCGGGGACTCGGGGGACGCCGCGCGCTGCCGTTCC
TCGGCAGCCCAGCCTGCGTAGACGGTCCCGCGGCGCTGACTGACCGGGC
TGTGCTTTCTCGTCTTCAGCACCAGCAGGCAGCTGGCTCCGGTTTTGGG
GTATCTGGGCTCCAGGCAGAAGCACAGCCTCCCCGACCTGCCCTACGAC
TACGGCGCCCTGGAACCTCACATCAACGCGCAGATCATGCAGCTGCACC
ACAGCAAGCACCACGCGGCCTACGTGAACAACCTGAACGTCACCGAGGA
GAAGTACCAGGAGGCGTTGGCCAAGGGTAGGTTCCAGGCTGAGCGGCGG
GAGGCAGTCCCCGGCAGAGGCGACCCCAGGGAGCCAGGCCCCATACGGA
CGGGCCTCTCCGTGGAGGAGAACTCGCTTCGTATTTGTACCGGTTCCGA
GTTTTCCAGGCACGATAGTCTCTCTTTTAAACACATGGTCTACCTCATT
GTAGAAGGAGTGCCTCGATGGGTTTGAACACACTTCTGTCATCTCAGGG
AACTTGGGGTCCTGCGAAGGAGCTTGCCTTACTGTTGTGAGCCACATTC
CGTTACACATATTGCCAGCACTGGTGAATTGTAGGGCCTGAAAAGAAAG
CTCTACTGTGTCACTCGTTTTTTTTGCAAATTGAAATTGTTCTTGTTGT
ATAATGTGCTTTGGGGAAATGTTTGGTCTCTCAGGTAGGTGTGCCAGCC
GTTTGCAGGAGGGCTGAGAGCGCCTGTCCACTGGTGGCCAGACATCATC
GGGTCCGCAGGTGTCTCTGAGTGTCAGGGTCACCTCCTGATAGAAGTGG
GAGTGGTGTCTTACTGCCAGGTCACACTGAAGGTGGGAGACAGGAGGAC
ACTACTCCGTGCTAGGAACCATGGTCCTTGTCATCTTCCTGAGAGCAAA
CGGGGTCGGGACTCCAGCCTAGGACTTGGAGACTCCCTGCATCCCCAGC
TACCCTGCAGGTGAAACTTCACTGAGCCCACTTGACTTCAGGTGGGAGA
GGAGGAGGCACACCGTTGTGGTGTGCGTTACCAAAAAAGCAGTAACCTA
ACTGTGGAACAGTTCAGGATCTTTAGACTTGATACTTTTCTTGATTTCC
CTTAGGGGTCAAAGTTCACAAGGAAAATAGCCCTTTAGTGGGAAACTGA
AACAGGCTTGTTTTTATTTACTATTTACTCATATTTGTAATGCAATTAA
AATATATGTTGTTAACTTTTTTTTTTTTTCTCAAGAGCTCGGTTGATAA
AACCAGGGGTATGTGGACTTTTTGAGTCTGTGCCTTTTGGGGGTGTGTA
TGGGAATGTTATGTTTTAACGTTTTTTACAAAATAGTATTGTCTTTTTA
ATTTATTCGTTAGTGGTTTGCACAAGGAAGATAATCGATAGTCATGTTT
TTTAGACTCTCTGTATTGCTTGGTAAGCTACGTAGTAAAAAATGTTTAC
TTTTCCTTAAATGTTTTGAATTTCGGGGTTATGAAATTTGTTGAGTAAT
TTTTAGACAGTCACATCTTGTTGACTGGAGGCATCTAGTGGAAAAATGC
AGTATTTCAGCCTGATTGTGTTTGAAGTAAATGATTAAAAGAGGAGGAA
GTTACCACATTCTGGAAGATTTACTTGAGACAGACGAACCTTGAATTAC
GGGAAAAGGCCCCGTGATTTAGGAAATAACAAATTTGGGAAACATGTAA
TGGGGAGAGACTGGGGAATACCCCAGTTGTGAAAGTACTTCCTGTAAGG
CAACATCTGACACCAGGAACCTTTCTCTTCAGTATTTTAAAAACAACTT
AATTTCAGTCCTTTACTTGTGGAATCAGAGCCTTACTTATGTAATACAA
CCCACTGGAAAAAAGCTTTTTATTGTATTGTACTATATTGTTTATAAGT
GATTGAGTACCTGCAGAGCTTTCTTTTACTTAAACATATTTTAAAAATT
ATTAAAAAGATTTTCATGTTTGAAAACTTGGGGAAAAAAGATAAAACAA
TGTCATAATCTCATCACATTTAACTGACTAATATGAATATTTTTCAACA
AAAAGCATTTGACTTTTATTATTATTTGAGGCAAAAACTAAACTGAAAC
CAAATAAAATGTTGAATTTCAGAAAAGTGTTCTTAAAAGCAGATATTAA
GCCCTCAATTTTTATTAATTTTAAAATGAGTAAGCAAACTGTAAGATGT
TAAGATTATTAAATTATCTCTCATTAAGCATGTATCTTTCCCATTTTAA
AAATTCAGTTACTATTGATTTATTAAAAAACAATAGAAAAGTTACATCT
ATTTTTAGAGTGACTCTCAAGAAAATTTAATAAGGGAAACACAGATCTA
TAAAAAACTGTTTGTAAACCTAACAAAGGAAAAAAAAAGTTGGTGTCTT
TAAAAAACCCTTTGAGATTAGCTGTGTATTCAGATATCAGTTGGTTCTC
TCCTTTCTGAATCGCTAAACAACTGGAGCTTGTTTCAGAAAGCAGCATT
GTCTACGTATGTTCCTTTTAAAAATTTTTTTTTTAATTCTGTCTAAAAA
AAACAGACGGGGTCACACTATGTTGCCCAGGCTGCTCTTGAATTCCTGG
GCTCCCCTATCCACGCACCTCGGCCTTCCAGAGTGCTGGGATTACAGGT
GTGAGCCACCGTGCCTAGTCAGACATACTTTTTTTTTTTTTTTTTTTTT
TGACGGAATCTCAGTCTGTCCCGCAGGTTGGAGTGCAGTGATGTGATCT
CGGCTCACTACAACCTCCGTCTCCTGGGTTCAAGCAATTCTCCTGCCTC
AGCCTCCTGAGTAGCTGGGATTACAGGCGTACGCCACCACGCCTGGCTA
CTTTTTGTGGGTTTTTTAGTAGAGACAGGGTTTTTCCATGTCGTCCAGG
CTGGTCTGGAACTCCTGACCTCAAGTGATCCGCCCATCTCAGCCTCCCA
AAGTGCTGGGATTACAGGCGTGAGCCACCTCACCTGGCCTTGTATGTAT
GTTTTTTTCTTTCTCTTTCTTTTTTTTTTTCTGTGACGGAGTATTGCCC
TGTCGCCCAGGCTGGAGTGCAGTGGCGTGTTCTTGGTTCACTGCAACCT
CTGCCTCCTGGGTTCAAGCCATTCTCCTGCCTCATGCTCCTGAGTAGCT
GGGATTATAGGCACGCGCCACCATACCTGGCTAATTTCTGTATTTTTAG
TGAAGACAGGGTTTCACCATGTTGGCCAGGCCGGTCTTGAAATCCTGAC
CTGAAGAGAGCTGCCCGCCTCGGCTTCCCAAAGTGCTGAGATTACAGGC
GTGAGCCACCGCACCCGGCCCAGACATATGTTCTTAACGTACACTTTCA
GAACAGCAGTTGAGAACTTTTTCCAAAATGTAAGCTCCTTGAGAGCAAA
GCTCTCAGTGGGTCTCTGTGTCCTTTACTTTGCTTCTCTTGGTATCTTG
TGGATAATATGCACAAAATATTTATTAAACAAGAGGATTTCCATTTTTT
TCTAATATTAATAGTTCATTTAGTCTGTAGCTTCTTGAAGGCATAGAAA
AGACTATTTTATTTTGCCTACTTATTTGGCAAGGTACCCATGCAGTATG
CCAGGAGTTACAGGGCCTATTGAGAAGGAGTAAAGGGTCTGGGAGGGGT
GGCAGGGAGTTGCTAATTCATGTATGGTGGCTAGAGAAGGCTTCACTGA
TGGGGTTGATGTTTCAGTGGAGACTTGAAGGAGGAACAGGAACGCAGCA
TGAGACTGTCTTGGGGAAGAGTGTTCAGCAGAGGCAGCAGTGCTCAGCT
AGCCTTGAGGGGAACATCCAAGTTGGTCATGTGAGTGCAAAAAGGCCAG
TGAGGCTGCAGCTGCGTGAGCACGTGCTCAATGACGGGGTCAAGTGCAG
CGCCTCTTGGTAGTAAGAGTAGCTGAGTGTAAACACCTAGGGTTCGCTC
CAGTGAGAAGGGGAGCCTTTGGAGGGGGCAAGGCCCACCCCCACCCCCC
AAAACTGAGGGGATAGTGGCACCAGTGCAGTGAGAAATACTGGTCCTCT
GTCCCAATGAGGTGGCAGTAGAGTTAGGGAGAAGGGATTAGGTTCTGCT
TGTGTTTTATTTTTTTCTTTTATTTTTCTCTGTGTGCTTTTATATAGAT
TGGATGTATTTTAAAGGTAGAGCTGGCTTTGCTAATGGATATGCTGGTG
GATTGGATGTGGGGTGAAGGGAGAGGAGTCAAAAATGACATCAGGTTCC
CAAGGATGTCCTGAGGGACAGAGCTGGCATTTACTGAGACGTGGAGGAC
AATAGGAGGAGTAAGTTTGGGGTAGAACATCAGGAGTTCAATTTTGAGT
ATGTTGAGTTTCTTGTTAATGTGACTTGGAGATATTTCCCAATGAATCG
TCGTCTGTCTTGAAAGTGAGAGTTGAAGGTTTTCTGTACAGTCTGTATG
TTGAGCCATACATTTTAGCATTCTATTATTTTGATTGTCCTCTTTGGTG
TTTCATTTCCTAAAATCTCTTCTATCTAAGAGATCCCTGAGCCTAGCAG
CCAGGAGTGAATTTTCTAGCTTTTCCACCTCTGCTAGGTCCTTCATCAG
TTGTTCAGCATGTCTGGGACAAATAATTTTGGGGCGGGGGGGTGCTGAA
TTTTAGGACAAACGGATGTTATAGATAAGCTGGTCCCATTATCTAATAG
CTTACAAAGAAAAAAATATAATGTATACAGTGGTTGAAAAAGTAGGAGT
TACAAAAAAATGTGTTTGCATTTTAACTTTTCAGGAGATGTTACAGCCC
AGATAGCTCTTCAGCCTGCACTGAAGTTCAATGGTGGTGGTCATATCAA
TCATAGCATTTTCTGGACAAACCTCAGCCCTAACGGTGGTGGAGAACCC
AAAGGTTGGTATATATTGGTGCACCCTTATCTACATTTTGTGCACAGTA
GGAATCGATTGTTGATTTACTGAAGTTACCTTATACTTGTTTTTTGTTT
TTTTTGTTGTTGTTTTTTTGAGACAAGAGTTTTGCTCTTGTTGCCCAGG
CTGGAGTACAGTGGCGCAGTCTTGGCTCACTGCAACCTCCGCTTCCTGG
GTTCAAGTGATTCTCCTGCCTCAGCCTCCCAAGTAGCTGGGATTACAGG
CGCCCGCCACCACGCCTGGCTAATTTTTGCATTTTTAGTAGAGATGAGG
TTTCATTCACCATGTTGGCCAGGCTGGTCTTGAACTCCTGACCTCATGA
TCTGCCCACCTTGGCCTCCCAAAGTGCTGGGATTACAGGCATGAGCCAC
CGTGCCTGGCCTAAGTTACCTTATTCTTACCAGAGGTATCATAGTGGCA
TATTTTTCATAACAACGAGTATGATAAAAGTTGTAACTTTCAAAAGGAC
ACAAGTAGGAATGGAGAATGCTTTCATGGGGTTATGTCATAAGATTTTA
TGTTCACGCCAACATAGAATATCACATCAACTCCACCTTCTGGTTTCTG
GATGGTACTTCATATTAGGGAAAGACTGAGAGACTTCATTTTATGAACT
TTTTTTTTTTGAGACGGAGTCTTACAGTCGCCCAGGCTGGAGCGCAGTG
GTGCAATGTCGGCTCATTGCAACCTCCGCCTTTCAGGTTCATGCAATTC
TTGTGTCTCAGCCACCCAAGTAGCTGGGATTACAGGCATGTGGCATCAC
TCCCGGCTAATTTTTATATTTTTGGTAGAGACAGGGTTTCACCATGTTG
GCGGGGCTGGTCTCGAACTCCTGGCCTCAAGTGATCCACCCACCTTGGC
CTCCCAAAGTGCTGTGGTATGGATGTGAGCCACCGCGCCAGTCTAGAAA
AGATGAACTTTCTGTCTACTAGTTTCTAGGGCCTGGATTAGATAGTTAA
CCGTCTGTACCTTTTTTCATCTATGTGATACAGGGATAACAATGGTGTT
GTTTTGAGAATTAAATTATAGTGTCATTTGCTATTTTTCACTCTTTCCT
AACTGATAAAAAGTCAGTTGTTCCAGGAAATTAGGTTTTCTGGGTATTT
TTAAAAAGTGTTCAGGGAATGAGGAGAGTACAGGGCTAGATGGTCTTGT
TCGCTATCACTGTTTCCTTGACTTCCTGTGTAGCTTGTGGAGGCCCAGA
TGACTTTAATTATCTTCTTCACCCAGGGAGGGCATGAGGCGTAAATGTA
GTTCTTGTCTCTTGTTTCTGGGCTCCGGATATGCACCCTAAGTTTCTGT
GGTTTTATTGTTCATTTTTAATCAGTGTTATGGAAGGCAGCCTATCTTA
TGCACTTGAAATAAGCAATTTCATTTTAAGGTAATGATGAATGAGGAGA
GTAAGCTTGGGAAACTGGGCAACTTTTTCTTTATTTTTGTTTTTATTTA
TTATTATTTTGGAGATGGAGTTTAGCTCTTTCGCCCAGGCTACAGTGAA
GTGGCTTGATCTTGGCTCACTGCAGCCTCTGCCCATCCCGGGTTCAAGT
GATTCTCTGCCTCAGCCTCCCGAGTAGCTGGGATTATAGGTGCCTGCCA
CCATGCCCGGCTAATTTTTTTGTATTTTTAGTAGAGAGGAGTTTCTCCA
TGTTGGTCAGGCTGGTTTTGAACTCCTGACCTCAGGTGATCCACCTGTC
TCGGCCTCCCAAAGTACTAAGATTACAGGTGTGAGCCACCGTGCCTGGC
CAACTTATTTTTTTTCTGAGTTCAGTCTTCTAGACTATCGGTTAATACT
TTTGAAGTTTTGTCAGTTACCAGAATATCAGATATATTCATATGCAACC
AGTGGTTTTGGTATGCTGCCATTTTTGTTTAATCTGTACCACATTCCAT
CATTTGTGCTTATAAAGATGATTACTTTTAAACCCGTAGAATAAGAGAA
GTGAGATTTTGACCCAGTTGATTTAGTTGATGTGACTAATCTAGAATTA
TATTCTGTATTACTACAGAATAAAAGGATTACAGCTCTTAAAGTATACC
CTTAGTTCAGTCGTAGAGAAAACTGCCCATCAAGACAGGAAGAGCTGGG
CTGCCTTGGAGAAGAACAATGACTTTATTCCCAAATGGAGGCGTAGAAG
AAGGAGGGTAGAAGTCAAAGGAAAATTTTCTATTCAGGGAAAAGAAAAA
GCTAACAGAAAGTAGTATTTCTTTCCTAACAAAGATAGTACAGTGAGTG
GGGAAATCCCTCTAACACGATTCATTAAAAAATGTGAACTACGCAGCTG
GAGGCCATTATCCTAAGCGAATTAACCCAGGAACAGAAAACCAAATACC
ACATGTCTCACTTGCAAGTGGAAGCTAAACATGGAGTACTTATGGATAT
AAAAATGGCAGCAACAGACACTGAGGACAGAGCAGGGAGGGAGAAAGAA
GGGGACGGGGGGTGAAAACTGTTGTATACTAAGCTCGCTACCTGGGTGA
TGGCATCAATTGCACCCCAGACCTCAGCATCATATGATACCCGTGTAAC
AAACCTGTGCGTGTACCCTCTGAATTTAAAATAAAATTTGAACCGGGCA
CGGTGGCTCACGCCTGTATTCCCAGCACTTTGGGAGGCCAAGGCGGGTG
GATCACCTGAGGTTGGGAGTTTGAGACCAGCCTGACCAACATGGAGAAA
CCCTGTCTCTACTAAAAACACAAAATTAGCCGGGCGTGATGGCACGTGC
CTGTAATCGCAGCTGCTCGGGAGGCTAAGGCAGGAGAATTGCTTGAACC
TGGGAGGCAGAGGTTGCAGTGACCCGAGATCATGGCATTGCACTCCAGC
CTGGGCAACAAGAGTGAAACTCCGTCTCAAAAGGTAAAAAATGAAAAAA
AAAAAAAAAAAAAAAAAAAGTTGAAGTTATAAAAAACAAAAACGTGAAT
TAAAATTGTGAAACTTAGATCCAGGTGTCGCATTCTGATGTTGTCTAAT
TTCTTGGGCCCTATGACAAAAATATTTTAATACATGTAATATAACATTT
TACTGTAATTATTGAAATCTGTTCATTTGTGGGTGGTTTTGGATTTTTT
TTTTAATAGGGGAGTTGCTGGAAGCCATCAAACGTGACTTTGGTTCCTT
TGACAAGTTTAAGGAGAAGCTGACGGCTGCATCTGTTGGTGTCCAAGGC
TCAGGTTGGGGTTGGCTTGGTTTCAATAAGGAACGGGGACACTTACAAA
TTGCTGCTTGTCCAAATCAGGATCCACTGCAAGGAACAACAGGTTAGAT
TTAAAAATTGTGATTTCATTTGGGAGAGATGCTCTACTGTAAAGCATTC
AACTAGAAATAAGGAAAACTAACAGTGTTTTAAGAACATGTAATAATTT
GCAAATCTTAACAGATAACACCCAGAGTCTTGTGTAAGTAAAAATGTTT
TAGAAGTCCTGATATTTCATAAAATTAAGTACACGTAATTTTATAGGTA
CATTAACAAACATGTTTTATTTTTATTTATTTTTTTTGAGACAGATCTC
ACTGTTGCCCAGGCTGGAGTGCAGTGGTGCGATCTCGGTTCACTGCAAC
CTCCGCCTCCTGGGTTCAAGTGATTCTCCTGCCTCAGCCTCCCAAATAG
CTGGGATTACAGGCACATACCACCATGCCCAGCTAATTTTTGTGTTTTT
AGTACAGACAGGGTTTCACCATGTTGGCCAGGCTGGTCTTGAACTCCTG
ACCTCAGGTATCTGCCTGCCTCGGCCTCCTGAAGTGCTGGGATTACAGG
CATGAGCCACCATGCCCAGCCAAATGTTTTACTTATTAACCCTGCTTCA
TTCTACAGAGAATTAATATCAGCTTAGATAATTATATGTACTAAAATAA
AAAAAAAATTAGCTATAGAACAAGAGTGAGGAAAAGAAATACAAGCAGA
GACTGATTTCAGAGCTTGTACTGTTACCTCCTCTGCTGAGCATTTGAGG
TTAAACTGGGACTAGATCTCAGGTTTCCAGACCTTCTGCCTTTCTGGGG
CTCTTTGGTAGAGTATATGAAAATATTCGGCTTGGAAGTGCCAGGCTGG
GCTCACCAAATACTTTGCTGTTGAGCCTAATTGGATTTAAAAAATGTCA
CTGAAGAGTATGGGAAATAGAGCCATGAGTTGTCTGGCAGAGGGTTGGG
ACTTTGAACTTAAAGTGATACCTTCACATCAACAACAGTCATTCTCTGC
CCCTTCTTCTACCCCTGACATTGGAATACTCTTGATCTGTGGCTCACTT
CACAGGATAGACCCAGATTTTGGCAGAATCTGAACAGCTCTTTTGGGAC
AGTGACATGGCATAACAATTTGTAAAAGCCACAAAAAATTTGTAAGCAC
CGATCTGTAGAGTCAGGTTATTTGTAGAGCTGCATGACCAGAAAGGCTG
ATATTTGTAAGTGAGTCCCAGCTACACTCCTTCCTCTCCCTGTGGAAAG
CACATTAGAAGGAAAATGAGGGAATGTCATGGCCTTTTGTTTATAAAAA
TCATTAATAAATTTTGCTACTTGAAATTATTAATAATAAATTACTAGTG
ACCTCCCTCACAACTGATCAAGACATACTGGTTAAGAATTGTTTTTGTA
GAGAGAGCTATAATTTATTTTGAGTGTTTTTGTGTGTGCGTGATGGAGT
CTCACTCTGTTGCCCAGGCTGGAGTGCAGTGGCGCAATCTTGGCTCACT
ACAACTTCCGCCTCCCAGGTTCAAGCAATTCTCCTGCGTCAGCTTCCTG
AGTAGCTGGGATTACAGGCGCCTGCCACCTCGCCAGGCTAATTTTTTGT
ATTTTTAGTAGAGGCGGGGTTTCACCATGTTGGCCAGGCTGGTCTCGAA
CGCCTGACCTCAAGAGATCTGCCTGCCTCGGCCTCCCAAAGTGCTGGGA
TTACAGGCCTGAGCCACTGCGCCCAGACCAAAAGTTTTTTTAAATGGAT
GTTTTACAGATAGAACTAAATTCTTCATTTACCTCACTTAATTTCTGAA
AACACTGTGTCCTCCAACTGCTCTGTATTCCCAAAGATTAAAATTAGCC
TTGGGGAGCAGAGCTGGTTCCTCGTAAACCCCTGTGAGTGCCCAGACCC
TTTGGAGGAGGTTCTCAGTGGGCTTTTGCCTGCTCCCACAGATGTAACC
CACCCTTCCTGCTGCTCCTAAGACCACCATGAGGAAGGCTGGGAATTGG
CTCTTGAATTAATTTGTTGGTATTTTTAAGAATAGCTATTAAAGTACTG
TTGTTAATTTTCCCAAGTCTATTTTATTGTTAGTATTTGTCACATTTTC
TTTGTAAACAGTGAATTAAAAATATTTTATAGGAATATTCATTCTTATC
TAGATTTCCTACTTATATAAAAAAACAAGTGAATGAGATTGTTACAAAG
GGTAATTTTGTGTGAGTAGAATAATAAAAGTTGAAATTGAGAAGATGCA
ATGTTTTAGACTGAAACTGATGGTTGGTTTGTTTTCCCCTTCTTTCTAA
CAGGCCTTATTCCACTGCTGGGGATTGATGTGTGGGAGCACGCTTACTA
CCTTCAGTATAAAAATGTCAGGCCTGATTATCTAAAAGCTATTTGGAAT
GTAATCAACTGGGAGAATGTAACTGAAAGATACATGGCTTGCAAAAAGT
AAACCACGATCGTTATGCTGAGTATGTTAAGCTCTTTATGACTGTTTTT
GTAGTGGTATAGAGTACTGCAGAATACAGTAAGCTGCTCTATTGTAGCA
TTTCTTGATGTTGCTTAGTCACTTATTTCATAAACAACTTAATGTTCTG
AATAATTTCTTACTAAACATTTTGTTATTGGGCAAGTGATTGAAAATAG
TAAATGCTTTGTGTGATTGAATCTGATTGGACATTTTCTTCAGAGAGCT
AAATTACAATTGTCATTTATAAAACCATCAAAAATATTCCATCCATATA
CTTTGGGGACTTGTAGGGATGCCTTTCTAGTCCTATTCTATTGCAGTTA
TAGAAAATCTAGTCTTTTGCCCCAGTTACTTAAAAATAAAATATTAACA
CTTTCCCAAGGGAAACACTCGGCTTTCTATAGAAAATTGCACTTTTTGT
CGAGTAATCCTCTGCAGTGATACTTCTGGTAGATGTCACCCAGTGGTTT
TTGTTAGGTCAAATGTTCCTGTATAGTTTTTGCAAATAGAGCTGTATAC
TGTTTAAATGTAGCAGGTGAACTGAACTGGGGTTTGCTCACCTGCACAG
TAAAGGCAAACTTCAACAGCAAAACTGCAAAAAGGTGGTTTTTGCAGTA
GGAGAAAGGAGGATGTTTATTTGCAGGGCGCCAAGCAAGGAGAATTGGG
CAGCTCATGCTTGAGACCCAATCTCCATGATGACCTACAAGCTAGAGTA
TTTAAAGGCAGTGGTAAATTTCAGGAAAGCAGAAGTTAAAGGCAAAATT
GTAAATCAGTCGAGATCGGGTGCCTTCAGGGTGGTATGGCTGTATACCA
AAATTGTAAATCACTACATGAAGCTTATATATTGGTTTGGCCTGAAAGG
TGAAGTGGGGTAGGCAGGGGGCGGGCTTACAGGTTATGGTGGATTCAAA
GACTCCCTGATTTGTGATTGGTTAAGGAAGCAAAGCTTTGTCTAAAAAC
TTGGGGTCCGCAGAAAGGAACATTAAGGTCTGGCCAGGCCCCTCAGGAA
GAAACTGAGAGCAAAGAATGGAGGTCAGAGTTTAGTCCCTGGTGTTCCC
CCTTATCTGACGTCTGTGTGAATCCATTTGGTGGGGGTCTGGGTTTCTG
AAAAGTAGCTCAGGGGCACGTGTTAAGGATGTCTCTAGGTGACTCTAAC
TTCCCTGGCTATTGTTTGAAACTGTTATGACCTTCTTGCTTATCAGCTT
GCTGGTTTCCTTCTCGGGGCGAGCTGGGTGCCTGGAGTTTTCGGTGAAG
GAAACTCAAGATTCTCCTTTATTTCTGTGCTTGTGGGAATCCCCCTGGC
ACACCCCAAAGAGGGGTCCCTGCTCCGTCTCACAGGGATCTTTTTGTAT
ATTTGGCTTAGCATCATACATTTGCCATGTTGTTTCATCATCTGCCTAA
TTTACTGTTTTTGAATATTTCATTTGTTTCTAATTGTTACTACAGATAA
TGCTGGGGTGAGCAACTCTGTGTACATAGGTTTATCTCCTATTGGAATA
TTTTCTTTATATAGGCGTTTTTTTTTTTTCTTTTTTTTTGGAGACAGAG
TCTTGCTCTGTTGCCCAGGCTGGAGTGCAGTGGCGCGACCGGAGCTCAC
TGCAACCTCCACTTCCCGGGTTCAAGTGATTGTCCCACCTCAGCCTCCT
GAATAGCTGGGATTACAGGTGCATGCTACCATGCCTGGCTACTTTTTGT
ATTTTTAGCAGAGACAGGGTTTCACCATGTTGGCCAGGGTGGTCTCGAA
CTCCTGACCTCAAGTGATCCGTCTGGCTCAGCCTCCCAAAGTGCTGGGA
TTACAGGTGTGAGCCACTGCACCTGGCCTATATAGGCTTTTTTCTTAAA
CCTATTTAGTAATGTTTTCCCAAGTTTATTTTTTATTTTTAATTTTTTC
CCCAAGTTTATTTTTCTATTTTTTTTTCATGGAAAAATGGGGTAACTTA
GCAGTTTCAATATTGAAGACTGAAGTTTAAAAAAAATTTAAATTCAAGG
TACTTTTAAAATTCAGTTAGAAAAGTAGGCTTTAAAAATTATTAGAGAC
AAGAGTACCAAAGCGGTGTGTGTATGTGTGTGTGTGTATGCATGCTTGT
GGATTGGAAAAACTTTGGAGACTGATTACTTTTCATTATATATGTGTCA
CAGTGAAACAGCTTTTATGTGTCATGTAAGATTACTGCTTGCCTCTCTA
AGGAAGGTCGTGACTGTTTAAATAGACGGGCAAGGTGGAACCTTTTGAA
AGATGAGCTTTTGAATATAAGTTGTCTGCTAGATCATGGTTTGTATTGA
ACTAACAAGGTTTGCAGATCTGCTGACTTATATAAAGCTTTTTGATTCC
TACTAAGCTTTAAGATTTAAAAAATGTTCAATGTTGAAATTTCTGTGGG
GCTCTATTTTTGCTTTGGCTTTCTGGTGAGAGAGTGAGGAAGCATTCTT
TCCTTCACTAAGTTTGTCTTTCTTGTCTTCTGGATAGATTGATTTTAAG
AGACTAAGGGAATTTACAAACTAAAGATTTTAGTCATCTGGTGGAAAAG
GAGACTTTAAGATTGTTTAGGGCTGGGCGGGGTGACTCACATCTGTAAT
CCCAGCACTTTGGGAGGCCAAGGCAGGCAGAACACTTGAAGGAGTTCAA
GACCAGCGTGGCCAACGTGGTGAAACCCTGTCTCTACTAAAAATACAAA
AATTGTTTAGCTCTGTTTTTCATAATAGAAATAGAAAAGGTAAAATTGC
TTTTCTTCTGAAAAGAACAAGTATTGTTCATCCAAGAAGGGTTTTTGTG
ACTGAATCAGCAGTGCCTGCCCTAGTCATAGCTGTGCTTCAAAAACCTC
AGCATGATTAGTGTTGGAGCAAAACAAGGAAGCAAAGCAAATACTGTTT
TTGAAATTCTATCTGTTGCTTGAACTATTTTGTAATAATTAAACTTTGA
TGTTGAGAAATCACAACTTTATTGTACACTTCATTGCAACTTGAAATTC
ATGGTCTTAAAGTGAGATTTGAATTTCTATTGAGCGCCTTTAAAAAAGT
AATACCAAACCATAAAGTTAAAATCTATGTATATTGAGTCATATCTAAA
ACCACGTATAAACATAAATTGTATTTCCTGTTTTAATTCCAGGGGAAGT
ACTGTTTGGGAAAGCTATTATTAGGTAAATGTTTTACAAATTACTGTTT
CTCACTTTCAGTCATACCCTAATGATCCCAGCAAGATAATGTCCTGTCT
TCTAAGATGTGCATCAAGCCTGGTACATACTGAAAACCCTATAAGGTCC
TGGATAATTTTTGTTTGATTATTCATTGAAGAAACATTTATTTTCCAAT
TGTGTGAAGTTTTTGACTGTTAATAAAAGAATCTGTCAACCATCAAAGA
GGTCTGCATTATGCTTGCATGTCAAAAACTTTAAAAATCCTATAATCTT
CTGTCATTTTCACTGAGTTTCCATGGGAAAGGAATAGTAAATAATGGGT
AGTTGAAATATTACTCTTAAGACCAAGACCTGTATCTCCAGTCATATCT
GTAATAACATCATCTGATAACCTAAAAGCATAGTATTAGGGATATACGA
CAAAACCAAAGTGTTTTTGCTGTTGTCACATACCACTCAATACTTTTAC
ACCAGGTTGTCCAGTGGACACCAGCTGGATGTCCTATAATTCAATTCTG
ACACTGTCTACCTGGAATTGGAGTCAGATCCCACAGGTGGAGGGCTCAG
CCCACAAGACTGCCCCCACTCCAGATACCAATCGCAAGTCCCTATACTT
CTGACTGGCTATAAAGTGGGGTTCCCGTGACCCTTTCCTTGGTTTTGAT
TTATTTGCTAGAGCAGCTCCCAGAACACAGAGAAACACTCTACTTATGT
TTACCCATTTTTAATTTTATTACTTTATTAAAACTACATATTGGTTACA
ATACTAATTTATTGTAAAGGATATTTCATTTATTTATTTTTTTATTTAG
AGATGGAGTCTCACTGTGTCACCCAGGCTGGAGTGCGGTGGCACAGTCT
CAACTCACTGCAACCTCCACCTCCTGAGTTCAAGCGATTCTCATGCCTC
AGCCTCCCAAGTAGCTGGGATTACAAGTGTGCACCACCATGCCCAGCTA
ATTTTTGTATTTTTAGTAGAGATGGGGTTTCACTCTGTTGGCCAGGCTG
GTCTCAAACTCCTGACCTCAAGTCATCCACCCACCTCAGCCTCCCAAAG
TGCTGAGATTACAGGTGTGAGCCATTGCCTCTGGCTTGTAAAGGATATT
TCAAAGAATACAGATGAACAGCCAGATGGAAGAGATGCACAGGGCAAGG
CATGTGGGAAGGGGTATGGAGCTTTCATGCTCTCTCCACGTCATCAGCT
ACCCAGAAGCTCATCTGGACCCCGACCCTTTGGGGTTTTACGGAGGCTT
CATTATGTCAGCATTACCATTGGCAATTGGCGATTAACTCAACCTTCAG
CCTCTCTCCCCTTTCTGGAGGTTGGGGGTTGAGGCTGGGAGTCCAAACC
CTCCAATCTTGCCTTGGTCTTTCTGGTAAGCAGCCCCCCGTCCTGAAGC
TACCTAGAGGCTCCCAGCCACCAATCATTTCATTGGCAAACCAAAGATA
CTTCAAGGGGTTTAGGAGCCAAGAGACCAAGCAAGGATTGAGACAATAT
ATATATTTCTTATAAATAACATGATCACAAATAGTATTTTATATATTTC
TCATAATAGCTGGTTGTGGTTACTCACACCTATCATCCCAGCTACTAAG
AAGCTAAGGCAGGAGGATCACAGGCCCAGGAGTTTGCAGCTGTAGTGAG
CTATGATTGTGCTACTGCACTCCAGCCTGGGTGACAGTGTGAGACCCTG
TCTCTTAAAGTTCTTGTAACATCTTAGAACATTAACCAACCACAGATTA
TTGCCTTAACTCTGAAAAATAAAGTAATCATACTTTTACAAGATGTATT
TTACATAAAGTTCTGGTGATATTTTACTCTGGTCATTGGAATATAGGAG
CTTACCACCTGGTACCACTCTTCCTAAAGTGGTATACTATTTGGTATAG
TACTGGCATACTATTTGGAAAGGGATGTGAAGTTGGCATGACCTGTCTA
GGGTGAAGTCATGCTGTCTGCTATTCATTGTGGGCTTTACAGTAATGAG
AGTGCTTCTGTTGTCAAGATGCTTCTGGGACACGTTAAAAAAAATTTAA
GATTTCTCAGTTTGTGAAAAGGAAGGAGCACGAATGCTTTCAAGTTCCT
ATGACATGCTAGTCATCTTTCAGTGTGTCCCATTGTTGCACCCAGTAGT
CTCGATGCCCAGCGTCACGGTGATTCCAAGTTTGCCTCTCTGCCCACTA
TAATTTGTGCTCCATGAAGTCCCAGGACATGAGGTTCT
SOD2 amino acid sequence (NP_001019636):
(SEQ ID NO: 5)
MLSRAVCGTSRQLAPVLGYLGSRQKHSLPDLPYDYGALEPHINAQIMQL
HHSKHHAAYVNNLNVTEEKYQEALAKGDVTAQIALQPALKFNGGGHINH
SIFWTNLSPNGGGEPKGELLEAIKRDFGSFDKFKEKLTAASVGVQGSGW
GWLGFNKERGHLQIAACPNQDPLQGTTGLIPLLGIDVWEHAYYLQYKNV
RPDYLKAIWNVINWENVTERYMACKK
Mitochondrial localization sequence derived
from SOD2 amino acid sequence:
(SEQ ID NO: 6)
MLSRAVCGTSRQLAPVLGYLGSRQKHSLPD
Mitochondrial localization sequence derived
from SOD2 nucleic acid sequence:
(SEQ ID NO: 7)
ATGTTGAGCCGGGCAGTGTGCGGCACCAGCAGGCAGCTGGCTCCGGTT
TTGGGGTATCTGGGCTCCAGGCAGAAGCACAGCCTCCCCGAC
SOD2 3’UTR nucleotide sequence:
(SEQ ID NO: 8)
ACCACGATCGTTATGCTGAGTATGTTAAGCTCTTTATGACTGTTTTTGT
AGTGGTATAGAGTACTGCAGAATACAGTAAGCTGCTCTATTGTAGCATT
TCTTGATGTTGCTTAGTCACTTATTTCATAAACAACTTAATGTTCTGAA
TAATTTCTTACTAAACATTTTGTTATTGGGCAAGTGATTGAAAATAGTA
AATGCTTTGTGTGATTGA
Nucleotide sequence of “BE4” APOBEC1-Cas9(D10A)-
2XUGI-bGHpoly(A) (Addgene100802
(Komor et al., 2017)):
(SEQ ID NO: 9)
gccgccaccatgagctcagagactggcccagtggctgtggaccccacat
tgagacggcggatcgagccccatgagtttgaggtattcttcgatccgag
agagctccgcaaggagacctgcctgctttacgaaattaattgggggggc
cggcactccatttggcgacatacatcacagaacactaacaagcacgtcg
aagtcaacttcatcgagaagttcacgacagaaagatatttctgtccgaa
cacaaggtgcagcattacctggtttctcagctggagcccatgcggcgaa
tgtagtagggccatcactgaattcctgtcaaggtatccccacgtcactc
tgtttatttacatcgcaaggctgtaccaccacgctgacccccgcaatcg
acaaggcctgcgggatttgatctcttcaggtgtgactatccaaattatg
actgagcaggagtcaggatactgctggagaaactttgtgaattatagcc
cgagtaatgaagcccactggcctaggtatccccatctgtgggtacgact
gtacgttcttgaactgtactgcatcatactgggcctgcctccttgtctc
aacattctgagaaggaagcagccacagctgacattctttaccatcgctc
ttcagtcttgtcattaccagcgactgcccccacacattctctgggccac
cgggttgaaatctggtggttcttctggtggttctagcggcagcgagact
cccgggacctcagagtccgccacacccgaaagttctggtggttcttctg
gtggttctgataaaaagtattctattggtttagccatcggcactaattc
cgttggatgggctgtcataaccgatgaatacaaagtaccttcaaagaaa
tttaaggtgttggggaacacagaccgtcattcgattaaaaagaatctta
tcggtgccctcctattcgatagtggcgaaacggcagaggcgactcgcct
gaaacgaaccgctcggagaaggtatacacgtcgcaagaaccgaatatgt
tacttacaagaaatttttagcaatgagatggccaaagttgacgattctt
tctttcaccgtttggaagagtccttccttgtcgaagaggacaagaaaca
tgaacggcaccccatctttggaaacatagtagatgaggtggcatatcat
gaaaagtacccaacgatttatcacctcagaaaaaagctagttgactcaa
ctgataaagcggacctgaggttaatctacttggctcttgcccatatgat
aaagttccgtgggcactttctcattgagggtgatctaaatccggacaac
tcggatgtcgacaaactgttcatccagttagtacaaacctataatcagt
tgtttgaagagaaccctataaatgcaagtggcgtggatgcgaaggctat
tcttagcgcccgcctctctaaatcccgacggctagaaaacctgatcgca
caattacccggagagaagaaaaatgggttgttcggtaaccttatagcgc
tctcactaggcctgacaccaaattttaagtcgaacttcgacttagctga
agatgccaaattgcagcttagtaaggacacgtacgatgacgatctcgac
aatctactggcacaaattggagatcagtatgcggacttatttttggctg
ccaaaaaccttagcgatgcaatcctcctatctgacatactgagagttaa
tactgagattaccaaggcgccgttatccgcttcaatgatcaaaaggtac
gatgaacatcaccaagacttgacacttctcaaggccctagtccgtcagc
aactgcctgagaaatataaggaaatattctttgatcagtcgaaaaacgg
gtacgcaggttatattgacggcggagcgagtcaagaggaattctacaag
tttatcaaacccatattagagaagatggatgggacggaagagttgcttg
taaaactcaatcgcgaagatctactgcgaaagcagcggactttcgacaa
cggtagcattccacatcaaatccacttaggcgaattgcatgctatactt
agaaggcaggaggatttttatccgttcctcaaagacaatcgtgaaaaga
ttgagaaaatcctaacctttcgcataccttactatgtgggacccctggc
ccgagggaactctcggttcgcatggatgacaagaaagtccgaagaaacg
attactccatggaattttgaggaagttgtcgataaaggtgcgtcagctc
aatcgttcatcgagaggatgaccaactttgacaagaatttaccgaacga
aaaagtattgcctaagcacagtttactttacgagtatttcacagtgtac
aatgaactcacgaaagttaagtatgtcactgagggcatgcgtaaacccg
cctttctaagcggagaacagaagaaagcaatagtagatctgttattcaa
gaccaaccgcaaagtgacagttaagcaattgaaagaggactactttaag
aaaattgaatgcttcgattctgtcgagatctccggggtagaagatcgat
ttaatgcgtcacttggtacgtatcatgacctcctaaagataattaaaga
taaggacttcctggataacgaagagaatgaagatatcttagaagatata
gtgttgactcttaccctctttgaagatcgggaaatgattgaggaaagac
taaaaacatacgctcacctgttcgacgataaggttatgaaacagttaaa
gaggcgtcgctatacgggctggggacgattgtcgcggaaacttatcaac
gggataagagacaagcaaagtggtaaaactattctcgattttctaaaga
gcgacggcttcgccaataggaactttatgcagctgatccatgatgactc
tttaaccttcaaagaggatatacaaaaggcacaggtttccggacaaggg
gactcattgcacgaacatattgcgaatcttgctggttcgccagccatca
aaaagggcatactccagacagtcaaagtagtggatgagctagttaaggt
catgggacgtcacaaaccggaaaacattgtaatcgagatggcacgcgaa
aatcaaacgactcagaaggggcaaaaaaacagtcgagagcggatgaaga
gaatagaagagggtattaaagaactgggcagccagatcttaaaggagca
tcctgtggaaaatacccaattgcagaacgagaaactttacctctattac
ctacaaaatggaagggacatgtatgttgatcaggaactggacataaacc
gtttatctgattacgacgtcgatcacattgtaccccaatcctttttgaa
ggacgattcaatcgacaataaagtgcttacacgctcggataagaaccga
gggaaaagtgacaatgttccaagcgaggaagtcgtaaagaaaatgaaga
actattggcggcagctcctaaatgcgaaactgataacgcaaagaaagtt
cgataacttaactaaagctgagaggggtggcttgtctgaacttgacaag
gccggatttattaaacgtcagctcgtggaaacccgccaaatcacaaagc
atgttgcacagatactagattcccgaatgaatacgaaatacgacgagaa
cgataagctgattcgggaagtcaaagtaatcactttaaagtcaaaattg
gtgtcggacttcagaaaggattttcaattctataaagttagggagataa
ataactaccaccatgcgcacgacgcttatcttaatgccgtcgtagggac
cgcactcattaagaaatacccgaagctagaaagtgagtttgtgtatggt
gattacaaagtttatgacgtccgtaagatgatcgcgaaaagcgaacagg
agataggcaaggctacagccaaatacttcttttattctaacattatgaa
tttctttaagacggaaatcactctggcaaacggagagatacgcaaacga
cctttaattgaaaccaatggggagacaggtgaaatcgtatgggataagg
gccgggacttcgcgacggtgagaaaagttttgtccatgccccaagtcaa
catagtaaagaaaactgaggtgcagaccggagggttttcaaaggaatcg
attcttccaaaaaggaatagtgataagctcatcgctcgtaaaaaggact
gggacccgaaaaagtacggtggcttcgatagccctacagttgcctattc
tgtcctagtagtggcaaaagttgagaagggaaaatccaagaaactgaag
tcagtcaaagaattattggggataacgattatggagcgctcgtcttttg
aaaagaaccccatcgacttccttgaggcgaaaggttacaaggaagtaaa
aaaggatctcataattaaactaccaaagtatagtctgtttgagttagaa
aatggccgaaaacggatgttggctagcgccggagagcttcaaaagggga
acgaactcgcactaccgtctaaatacgtgaatttcctgtatttagcgtc
ccattacgagaagttgaaaggttcacctgaagataacgaacagaagcaa
ctttttgttgagcagcacaaacattatctcgacgaaatcatagagcaaa
tttcggaattcagtaagagagtcatcctagctgatgccaatctggacaa
agtattaagcgcatacaacaagcacagggataaacccatacgtgagcag
gcggaaaatattatccatttgtttactcttaccaacctcggcgctccag
ccgcattcaagtattttgacacaacgatagatcgcaaacgatacacttc
taccaaggaggtgctagacgcgacactgattcaccaatccatcacggga
ttatatgaaactcggatagatttgtcacagcttgggggtgactctggtg
gttctggaggatctggtggttctactaatctgtcagatattattgaaaa
ggagaccggtaagcaactggttatccaggaatccatcctcatgctccca
gaggaggtggaagaagtcattgggaacaagccggaaagcgatatactcg
tgcacaccgcctacgacgagagcaccgacgagaatgtcatgcttctgac
tagcgacgcccctgaatacaagccttgggctctggtcatacaggatagc
aacggtgagaacaagattaagatgctctctggtggttctggaggatctg
gtggttctactaatctgtcagatattattgaaaaggagaccggtaagca
actggttatccaggaatccatcctcatgctcccagaggaggtggaagaa
gtcattgggaacaagccggaaagcgatatactcgtgcacaccgcctacg
acgagagcaccgacgagaatgtcatgcttctgactagcgacgcccctga
atacaagccttgggctctggtcatacaggatagcaacggtgagaacaag
attaagatgctctctggtggttctcccaagaagaagaggaaagtctaac
cggtcatcatcaccatcaccattgagtttaaacccgctgatcagcctcg
actgtgccttctagttgccagccatctgttgtttgcccctcccccgtgc
cttccttgaccctggaaggtgccactcccactgtcctttcctaataaaa
tgaggaaattgcatcgcattgtctgagtaggtgtcattctattctgggg
ggtggggtggggcaggacagcaagggggaggattgggaagacaatagca
ggcatgctggggatgcggtgggctctatgg
Nucleotide sequence of COX8-APOBEC1-
Cas9(D10A)-2XUGI-bGHpoly(A):
(SEQ ID NO: 10)
GCCACCATGTCCGTCCTGACGCCGCTGCTGCTGCGGGGCTTGACAGGCT
CGGCCCGGCGGCTCCCAGTGCCGCGCGCCAAGATCCATTCGTTGGATCC
Gagctcagagactggcccagtggctgtggaccccacattgagacggcgg
atcgagccccatgagtttgaggtattcttcgatccgagagagctccgca
aggagacctgecctgetttacgaaattaattgggggggccggcactcca
tttggcgacatacatcacagaacactaacaagcacgtcgaagtcaactt
catcgagaagttcacgacagaaagatatttctgtccgaacacaaggtgc
agcattacctggtttctcagctggagcccatgcggcgaatgtagtaggg
ccatcactgaattcctgtcaaggtatccccacgtcactctgtttattta
catcgcaaggctgtaccaccacgctgacccccgcaatcgacaaggcctg
cgggatttgatctcttcaggtgtgactatccaaattatgactgagcagg
agtcaggatactgctggagaaactttgtgaattatagcccgagtaatga
agcccactggcctaggtatccccatctgtgggtacgactgtacgttctt
gaactgtactgcatcatactgggcctgcctccttgtctcaacattctga
gaaggaagcagccacagctgacattctttaccatcgctcttcagtcttg
tcattaccagcgactgcccccacacattctctgggccaccgggttgaaa
tctggtggttcttctggtggttctagcggcagcgagactcccgggacct
cagagtccgccacacccgaaagttctggtggttcttctggtggttctga
taaaaagtattctattggtttagccatcggcactaattccgttggatgg
gctgtcataaccgatgaatacaaagtaccttcaaagaaatttaaggtgt
tggggaacacagaccgtcattcgattaaaaagaatcttatcggtgccct
cctattcgatagtggcgaaacggcagaggcgactcgcctgaaacgaacc
gctcggagaaggtatacacgtcgcaagaaccgaatatgttacttacaag
aaatttttagcaatgagatggccaaagttgacgattctttctttcaccg
tttggaagagtccttccttgtcgaagaggacaagaaacatgaacggcac
cccatctttggaaacatagtagatgaggtggcatatcatgaaaagtacc
caacgatttatcacctcagaaaaaagctagttgactcaactgataaagc
ggacctgaggttaatctacttggctcttgcccatatgataaagttccgt
gggcactttctcattgagggtgatctaaatccggacaactcggatgtcg
acaaactgttcatccagttagtacaaacctataatcagttgtttgaaga
gaaccctataaatgcaagtggcgtggatgcgaaggctattcttagcgcc
cgcctctctaaatcccgacggctagaaaacctgatcgcacaattacccg
gagagaagaaaaatgggttgttcggtaaccttatagcgctctcactagg
cctgacaccaaattttaagtcgaacttcgacttagctgaagatgccaaa
ttgcagcttagtaaggacacgtacgatgacgatctcgacaatctactgg
cacaaattggagatcagtatgcggacttatttttggctgccaaaaacct
tagcgatgcaatcctcctatctgacatactgagagttaatactgagatt
accaaggcgccgttatccgcttcaatgatcaaaaggtacgatgaacatc
accaagacttgacacttctcaaggccctagtccgtcagcaactgcctga
gaaatataaggaaatattctttgatcagtcgaaaaacgggtacgcaggt
tatattgacggcggagcgagtcaagaggaattctacaagtttatcaaac
ccatattagagaagatggatgggacggaagagttgcttgtaaaactcaa
tcgcgaagatctactgcgaaagcagcggactttcgacaacggtagcatt
ccacatcaaatccacttaggcgaattgcatgctatacttagaaggcagg
aggatttttatccgttcctcaaagacaatcgtgaaaagattgagaaaat
cctaacctttcgcataccttactatgtgggacccctggcccgagggaac
tctcggttcgcatggatgacaagaaagtccgaagaaacgattactccat
ggaattttgaggaagttgtcgataaaggtgcgtcagctcaatcgttcat
cgagaggatgaccaactttgacaagaatttaccgaacgaaaaagtattg
cctaagcacagtttactttacgagtatttcacagtgtacaatgaactca
cgaaagttaagtatgtcactgagggcatgcgtaaacccgcctttctaag
cggagaacagaagaaagcaatagtagatctgttattcaagaccaaccgc
aaagtgacagttaagcaattgaaagaggactactttaagaaaattgaat
gcttcgattctgtcgagatctccggggtagaagatcgatttaatgcgtc
acttggtacgtatcatgacctcctaaagataattaaagataaggacttc
ctggataacgaagagaatgaagatatcttagaagatatagtgttgactc
ttaccctctttgaagatcgggaaatgattgaggaaagactaaaaacata
cgctcacctgttcgacgataaggttatgaaacagttaaagaggcgtcgc
tatacgggctggggacgattgtcgcggaaacttatcaacgggataagag
acaagcaaagtggtaaaactattctcgattttctaaagagcgacggctt
cgccaataggaactttatgcagctgatccatgatgactctttaaccttc
aaagaggatatacaaaaggcacaggtttccggacaaggggactcattgc
acgaacatattgcgaatcttgctggttcgccagccatcaaaaagggcat
actccagacagtcaaagtagtggatgagctagttaaggtcatgggacgt
cacaaaccggaaaacattgtaatcgagatggcacgcgaaaatcaaacga
ctcagaaggggcaaaaaaacagtcgagagcggatgaagagaatagaaga
gggtattaaagaactgggcagccagatcttaaaggagcatcctgtggaa
aatacccaattgcagaacgagaaactttacctctattacctacaaaatg
gaagggacatgtatgttgatcaggaactggacataaaccgtttatctga
ttacgacgtcgatcacattgtaccccaatcctttttgaaggacgattca
atcgacaataaagtgcttacacgctcggataagaaccgagggaaaagtg
acaatgttccaagcgaggaagtcgtaaagaaaatgaagaactattggcg
gcagctcctaaatgcgaaactgataacgcaaagaaagttcgataactta
actaaagctgagaggggtggcttgtctgaacttgacaaggccggattta
ttaaacgtcagctcgtggaaacccgccaaatcacaaagcatgttgcaca
gatactagattcccgaatgaatacgaaatacgacgagaacgataagctg
attcgggaagtcaaagtaatcactttaaagtcaaaattggtgtcggact
tcagaaaggattttcaattctataaagttagggagataaataactacca
ccatgcgcacgacgcttatcttaatgccgtcgtagggaccgcactcatt
aagaaatacccgaagctagaaagtgagtttgtgtatggtgattacaaag
tttatgacgtccgtaagatgatcgcgaaaagcgaacaggagataggcaa
ggctacagccaaatacttcttttattctaacattatgaatttctttaag
acggaaatcactctggcaaacggagagatacgcaaacgacctttaattg
aaaccaatggggagacaggtgaaatcgtatgggataagggccgggactt
cgcgacggtgagaaaagttttgtccatgccccaagtcaacatagtaaag
aaaactgaggtgcagaccggagggttttcaaaggaatcgattcttccaa
aaaggaatagtgataagctcatcgctcgtaaaaaggactgggacccgaa
aaagtacggtggcttcgatagccctacagttgcctattctgtcctagta
gtggcaaaagttgagaagggaaaatccaagaaactgaagtcagtcaaag
aattattggggataacgattatggagcgctcgtcttttgaaaagaaccc
catcgacttccttgaggcgaaaggttacaaggaagtaaaaaaggatctc
ataattaaactaccaaagtatagtctgtttgagttagaaaatggccgaa
aacggatgttggctagcgccggagagcttcaaaaggggaacgaactcgc
actaccgtctaaatacgtgaatttcctgtatttagcgtcccattacgag
aagttgaaaggttcacctgaagataacgaacagaagcaactttttgttg
agcagcacaaacattatctcgacgaaatcatagagcaaatttcggaatt
cagtaagagagtcatcctagctgatgccaatctggacaaagtattaagc
gcatacaacaagcacagggataaacccatacgtgagcaggcggaaaata
ttatccatttgtttactcttaccaacctcggcgctccagccgcattcaa
gtattttgacacaacgatagatcgcaaacgatacacttctaccaaggag
gtgctagacgcgacactgattcaccaatccatcacgggattatatgaaa
ctcggatagatttgtcacagcttgggggtgactctggtggttctggagg
atctggtggttctactaatctgtcagatattattgaaaaggagaccggG
aagcaactggttatccaggaatccatcctcatgctcccagaggaggtgg
aagaagtcattgggaacaagccggaaagcgatatactcgtgcacaccgc
ctacgacgagagcaccgacgagaatgtcatgcttctgactagcgacgcc
cctgaatacaagccttgggctctggtcatacaggatagcaacggtgaga
acaagattaagatgctctctggtggttctggaggatctggtggttctac
taatctgtcagatattattgaaaaggagaccggGaagcaactggttatc
caggaatccatcctcatgctcccagaggaggtggaagaagtcattggga
acaagccggaaagcgatatactcgtgcacaccgcctacgacgagagcac
cgacgagaatgtcatgcttctgactagcgacgcccctgaatacaagcct
tgggctctggtcatacaggatagcaacggtgagaacaagattaagatgc
tctctggtggttctggaggatctggtggttctGCTAGCGACTATAAGGA
CCACGACGGAGACTACAAGGATCATGATATTGATTACAAAGACGATGAC
GATAAGTAAgtttaaacccgctgatcagcctcgactgtgccttctagtt
gccagccatctgttgtttgcccctcccccgtgccttccttgaccctgga
aggtgccactcccactgtcctttcctaataaaatgaggaaattgcatcg
cattgtctgagtaggtgtcattctattctggggggtggggtggggcagg
acagcaagggggaggattgggaagacaatagcaggcatgctggggatgc
ggtgggctctatgg
Nucleotide sequence of COX8-APOBECl-Cas9
(D10A)-2XUGI-RevNES-bGHpoly(A):
(SEQ ID NO: 11)
GCCACCATGTCCGTCCTGACGCCGCTGCTGCTGCGGGGCTTGACAGGCT
CGGCCCGGCGGCTCCCAGTGCCGCGCGCCAAGATCCATTCGTTGGATCC
Gagctcagagactggcccagtggctgtggaccccacattgagacggcgg
atcgagccccatgagtttgaggtattcttcgatccgagagagctccgca
aggagacctgcctgctttacgaaattaattgggggggccggcactccat
ttggcgacatacatcacagaacactaacaagcacgtcgaagtcaacttc
atcgagaagttcacgacagaaagatatttctgtccgaacacaaggtgca
gcattacctggtttctcagctggagcccatgcggcgaatgtagtagggc
catcactgaattcctgtcaaggtatccccacgtcactctgtttatttac
atcgcaaggctgtaccaccacgctgacccccgcaatcgacaaggcctgc
gggatttgatctcttcaggtgtgactatccaaattatgactgagcagga
gtcaggatactgctggagaaactttgtgaattatagcccgagtaatgaa
gcccactggcctaggtatccccatctgtgggtacgactgtacgttcttg
aactgtactgcatcatactgggcctgcctccttgtctcaacattctgag
aaggaagcagccacagctgacattctttaccatcgctcttcagtcttgt
cattaccagcgactgcccccacacattctctgggccaccgggttgaaat
ctggtggttcttctggtggttctagcggcagcgagactcccgggacctc
agagtccgccacacccgaaagttctggtggttcttctggtggttctgat
aaaaagtattctattggtttagccatcggcactaattccgttggatggg
ctgtcataaccgatgaatacaaagtaccttcaaagaaatttaaggtgtt
ggggaacacagaccgtcattcgattaaaaagaatcttatcggtgccctc
ctattcgatagtggcgaaacggcagaggcgactcgcctgaaacgaaccg
ctcggagaaggtatacacgtcgcaagaaccgaatatgttacttacaaga
aatttttagcaatgagatggccaaagttgacgattctttctttcaccgt
ttggaagagtccttccttgtcgaagaggacaagaaacatgaacggcacc
ccatctttggaaacatagtagatgaggtggcatatcatgaaaagtaccc
aacgatttatcacctcagaaaaaagctagttgactcaactgataaagcg
gacctgaggttaatctacttggctcttgcccatatgataaagttccgtg
ggcactttctcattgagggtgatctaaatccggacaactcggatgtcga
caaactgttcatccagttagtacaaacctataatcagttgtttgaagag
aaccctataaatgcaagtggcgtggatgcgaaggctattcttagcgccc
gcctctctaaatcccgacggctagaaaacctgatcgcacaattacccgg
agagaagaaaaatgggttgttcggtaaccttatagcgctctcactaggc
ctgacaccaaattttaagtcgaacttcgacttagctgaagatgccaaat
tgcagcttagtaaggacacgtacgatgacgatctcgacaatctactggc
acaaattggagatcagtatgcggacttatttttggctgccaaaaacctt
agcgatgcaatcctcctatctgacatactgagagttaatactgagatta
ccaaggcgccgttatccgcttcaatgatcaaaaggtacgatgaacatca
ccaagacttgacacttctcaaggccctagtccgtcagcaactgcctgag
aaatataaggaaatattctttgatcagtcgaaaaacgggtacgcaggtt
atattgacggcggagcgagtcaagaggaattctacaagtttatcaaacc
catattagagaagatggatgggacggaagagttgcttgtaaaactcaat
cgcgaagatctactgcgaaagcagcggactttcgacaacggtagcattc
cacatcaaatccacttaggcgaattgcatgctatacttagaaggcagga
ggatttttatccgttcctcaaagacaatcgtgaaaagattgagaaaatc
ctaacctttcgcataccttactatgtgggacccctggcccgagggaact
ctcggttcgcatggatgacaagaaagtccgaagaaacgattactccatg
gaattttgaggaagttgtcgataaaggtgcgtcagctcaatcgttcatc
gagaggatgaccaactttgacaagaatttaccgaacgaaaaagtattgc
ctaagcacagtttactttacgagtatttcacagtgtacaatgaactcac
gaaagttaagtatgtcactgagggcatgcgtaaacccgcctttctaagc
ggagaacagaagaaagcaatagtagatctgttattcaagaccaaccgca
aagtgacagttaagcaattgaaagaggactactttaagaaaattgaatg
cttcgattctgtcgagatctccggggtagaagatcgatttaatgcgtca
cttggtacgtatcatgacctcctaaagataattaaagataaggacttcc
tggataacgaagagaatgaagatatcttagaagatatagtgttgactct
taccctctttgaagatcgggaaatgattgaggaaagactaaaaacatac
gctcacctgttcgacgataaggttatgaaacagttaaagaggcgtcgct
atacgggctggggacgattgtcgcggaaacttatcaacgggataagaga
caagcaaagtggtaaaactattctcgattttctaaagagcgacggcttc
gccaataggaactttatgcagctgatccatgatgactctttaaccttca
aagaggatatacaaaaggcacaggtttccggacaaggggactcattgca
cgaacatattgcgaatcttgctggttcgccagccatcaaaaagggcata
ctccagacagtcaaagtagtggatgagctagttaaggtcatgggacgtc
acaaaccggaaaacattgtaatcgagatggcacgcgaaaatcaaacgac
tcagaaggggcaaaaaaacagtcgagagcggatgaagagaatagaagag
ggtattaaagaactgggcagccagatcttaaaggagcatcctgtggaaa
atacccaattgcagaacgagaaactttacctctattacctacaaaatgg
aagggacatgtatgttgatcaggaactggacataaaccgtttatctgat
tacgacgtcgatcacattgtaccccaatcctttttgaaggacgattcaa
tcgacaataaagtgcttacacgctcggataagaaccgagggaaaagtga
caatgttccaagcgaggaagtcgtaaagaaaatgaagaactattggcgg
cagctcctaaatgcgaaactgataacgcaaagaaagttcgataacttaa
ctaaagctgagaggggtggcttgtctgaacttgacaaggccggatttat
taaacgtcagctcgtggaaacccgccaaatcacaaagcatgttgcacag
atactagattcccgaatgaatacgaaatacgacgagaacgataagctga
ttcgggaagtcaaagtaatcactttaaagtcaaaattggtgtcggactt
cagaaaggattttcaattctataaagttagggagataaataactaccac
catgcgcacgacgcttatcttaatgccgtcgtagggaccgcactcatta
agaaatacccgaagctagaaagtgagtttgtgtatggtgattacaaagt
ttatgacgtccgtaagatgatcgcgaaaagcgaacaggagataggcaag
gctacagccaaatacttcttttattctaacattatgaatttctttaaga
cggaaatcactctggcaaacggagagatacgcaaacgacctttaattga
aaccaatggggagacaggtgaaatcgtatgggataagggccgggacttc
gcgacggtgagaaaagttttgtccatgccccaagtcaacatagtaaaga
aaactgaggtgcagaccggagggttttcaaaggaatcgattcttccaaa
aaggaatagtgataagctcatcgctcgtaaaaaggactgggacccgaaa
aagtacggtggcttcgatagccctacagttgcctattctgtcctagtag
tggcaaaagttgagaagggaaaatccaagaaactgaagtcagtcaaaga
attattggggataacgattatggagcgctcgtcttttgaaaagaacccc
atcgacttccttgaggcgaaaggttacaaggaagtaaaaaaggatctca
taattaaactaccaaagtatagtctgtttgagttagaaaatggccgaaa
acggatgttggctagcgccggagagcttcaaaaggggaacgaactcgca
ctaccgtctaaatacgtgaatttcctgtatttagcgtcccattacgaga
agttgaaaggttcacctgaagataacgaacagaagcaactttttgttga
gcagcacaaacattatctcgacgaaatcatagagcaaatttcggaattc
agtaagagagtcatcctagctgatgccaatctggacaaagtattaagcg
catacaacaagcacagggataaacccatacgtgagcaggcggaaaatat
tatccatttgtttactcttaccaacctcggcgctccagccgcattcaag
tattttgacacaacgatagatcgcaaacgatacacttctaccaaggagg
tgctagacgcgacactgattcaccaatccatcacgggattatatgaaac
tcggatagatttgtcacagcttgggggtgactctggtggttctggagga
tctggtggttctactaatctgtcagatattattgaaaaggagaccggGa
agcaactggttatccaggaatccatcctcatgctcccagaggaggtgga
agaagtcattgggaacaagccggaaagcgatatactcgtgcacaccgcc
tacgacgagagcaccgacgagaatgtcatgcttctgactagcgacgccc
ctgaatacaagccttgggctctggtcatacaggatagcaacggtgagaa
caagattaagatgctctctggtggttctggaggatctggtggttctact
aatctgtcagatattattgaaaaggagaccggGaagcaactggttatcc
aggaatccatcctcatgctcccagaggaggtggaagaagtcattgggaa
caagccggaaagcgatatactcgtgcacaccgcctacgacgagagcacc
gacgagaatgtcatgcttctgactagcgacgcccctgaatacaagcctt
gggctctggtcatacaggatagcaacggtgagaacaagattaagatgct
ctctggtggttctggaggatctggtggttctCTTCAGCTACCACCGATT
GAGAGACTTACTCTTGCTAGCGACTATAAGGACCACGACGGAGACTACA
AGGATCATGATATTGATTACAAAGACGATGACGATAAGTAAgtttaaac
ccgctgatcagcctcgactgtgccttctagttgccagccatctgttgtt
tgcccctcccccgtgccttccttgaccctggaaggtgccactcccactg
tcctttcctaataaaatgaggaaattgcatcgcattgtctgagtaggtg
tcattctattctggggggtggggtggggcaggacagcaagggggaggat
tgggaagacaatagcaggcatgctggggatgcggtgggctctatgg.
Nucleotide sequence of BE4-SOD2MTS3 UTR:
(SEQ ID NO: 12)
atgttgagccgggcagtgtgcggcaccagcaggcagctggctccggttt
tggggtatctgggctccaggcagaagcacagcctccccgacGATCCGag
ctcagagactggcccagtggctgtggaccccacattgagacggcggatc
gagccccatgagtttgaggtattcttcgatccgagagagctccgcaagg
agacctgcctgctttacgaaattaattgggggggccggcactccatttg
gcgacatacatcacagaacactaacaagcacgtcgaagtcaacttcatc
gagaagttcacgacagaaagatatttctgtccgaacacaaggtgcagca
ttacctggtttctcagctggagcccatgcggcgaatgtagtagggccat
cactgaattcctgtcaaggtatccccacgtcactctgtttatttacatc
gcaaggctgtaccaccacgctgacccccgcaatcgacaaggcctgcggg
atttgatctcttcaggtgtgactatccaaattatgactgagcaggagtc
aggatactgctggagaaactttgtgaattatagcccgagtaatgaagcc
cactggcctaggtatccccatctgtgggtacgactgtacgttcttgaac
tgtactgcatcatactgggcctgcctccttgtctcaacattctgagaag
gaagcagccacagctgacattctttaccatcgctcttcagtcttgtcat
taccagcgactgcccccacacattctctgggccaccgggttgaaatctg
gtggttcttctggtggttctagcggcagcgagactcccgggacctcaga
gtccgccacacccgaaagttctggtggttcttctggtggttctgataaa
aagtattctattggtttagccatcggcactaattccgttggatgggctg
tcataaccgatgaatacaaagtaccttcaaagaaatttaaggtgttggg
gaacacagaccgtcattcgattaaaaagaatcttatcggtgccctccta
ttcgatagtggcgaaacggcagaggcgactcgcctgaaacgaaccgctc
ggagaaggtatacacgtcgcaagaaccgaatatgttacttacaagaaat
ttttagcaatgagatggccaaagttgacgattctttctttcaccgtttg
gaagagtccttccttgtcgaagaggacaagaaacatgaacggcacccca
tctttggaaacatagtagatgaggtggcatatcatgaaaagtacccaac
gatttatcacctcagaaaaaagctagttgactcaactgataaagcggac
ctgaggttaatctacttggctcttgcccatatgataaagttccgtgggc
actttctcattgagggtgatctaaatccggacaactcggatgtcgacaa
actgttcatccagttagtacaaacctataatcagttgtttgaagagaac
cctataaatgcaagtggcgtggatgcgaaggctattcttagcgcccgcc
tctctaaatcccgacggctagaaaacctgatcgcacaattacccggaga
gaagaaaaatgggttgttcggtaaccttatagcgctctcactaggcctg
acaccaaattttaagtcgaacttcgacttagctgaagatgccaaattgc
agcttagtaaggacacgtacgatgacgatctcgacaatctactggcaca
aattggagatcagtatgcggacttatttttggctgccaaaaaccttagc
gatgcaatcctcctatctgacatactgagagttaatactgagattacca
aggcgccgttatccgcttcaatgatcaaaaggtacgatgaacatcacca
agacttgacacttctcaaggccctagtccgtcagcaactgcctgagaaa
tataaggaaatattctttgatcagtcgaaaaacgggtacgcaggttata
ttgacggcggagcgagtcaagaggaattctacaagtttatcaaacccat
attagagaagatggatgggacggaagagttgcttgtaaaactcaatcgc
gaagatctactgcgaaagcagcggactttcgacaacggtagcattccac
atcaaatccacttaggcgaattgcatgctatacttagaaggcaggagga
tttttatccgttcctcaaagacaatcgtgaaaagattgagaaaatccta
acctttcgcataccttactatgtgggacccctggcccgagggaactctc
ggttcgcatggatgacaagaaagtccgaagaaacgattactccatggaa
ttttgaggaagttgtcgataaaggtgcgtcagctcaatcgttcatcgag
aggatgaccaactttgacaagaatttaccgaacgaaaaagtattgccta
agcacagtttactttacgagtatttcacagtgtacaatgaactcacgaa
agttaagtatgtcactgagggcatgcgtaaacccgcctttctaagcgga
gaacagaagaaagcaatagtagatctgttattcaagaccaaccgcaaag
tgacagttaagcaattgaaagaggactactttaagaaaattgaatgctt
cgattctgtcgagatctccggggtagaagatcgatttaatgcgtcactt
ggtacgtatcatgacctcctaaagataattaaagataaggacttcctgg
ataacgaagagaatgaagatatcttagaagatatagtgttgactcttac
cctctttgaagatcgggaaatgattgaggaaagactaaaaacatacgct
cacctgttcgacgataaggttatgaaacagttaaagaggcgtcgctata
cgggctggggacgattgtcgcggaaacttatcaacgggataagagacaa
gcaaagtggtaaaactattctcgattttctaaagagcgacggcttcgcc
aataggaactttatgcagctgatccatgatgactctttaaccttcaaag
aggatatacaaaaggcacaggtttccggacaaggggactcattgcacga
acatattgcgaatcttgctggttcgccagccatcaaaaagggcatactc
cagacagtcaaagtagtggatgagctagttaaggtcatgggacgtcaca
aaccggaaaacattgtaatcgagatggcacgcgaaaatcaaacgactca
gaaggggcaaaaaaacagtcgagagcggatgaagagaatagaagagggt
attaaagaactgggcagccagatcttaaaggagcatcctgtggaaaata
cccaattgcagaacgagaaactttacctctattacctacaaaatggaag
ggacatgtatgttgatcaggaactggacataaaccgtttatctgattac
gacgtcgatcacattgtaccccaatcctttttgaaggacgattcaatcg
acaataaagtgcttacacgctcggataagaaccgagggaaaagtgacaa
tgttccaagcgaggaagtcgtaaagaaaatgaagaactattggcggcag
ctcctaaatgcgaaactgataacgcaaagaaagttcgataacttaacta
aagctgagaggggtggcttgtctgaacttgacaaggccggatttattaa
acgtcagctcgtggaaacccgccaaatcacaaagcatgttgcacagata
ctagattcccgaatgaatacgaaatacgacgagaacgataagctgattc
gggaagtcaaagtaatcactttaaagtcaaaattggtgtcggacttcag
aaaggattttcaattctataaagttagggagataaataactaccaccat
gcgcacgacgcttatcttaatgccgtcgtagggaccgcactcattaaga
aatacccgaagctagaaagtgagtttgtgtatggtgattacaaagttta
tgacgtccgtaagatgatcgcgaaaagcgaacaggagataggcaaggct
acagccaaatacttcttttattctaacattatgaatttctttaagacgg
aaatcactctggcaaacggagagatacgcaaacgacctttaattgaaac
caatggggagacaggtgaaatcgtatgggataagggccgggacttcgcg
acggtgagaaaagttttgtccatgccccaagtcaacatagtaaagaaaa
ctgaggtgcagaccggagggttttcaaaggaatcgattcttccaaaaag
gaatagtgataagctcatcgctcgtaaaaaggactgggacccgaaaaag
tacggtggcttcgatagccctacagttgcctattctgtcctagtagtgg
caaaagttgagaagggaaaatccaagaaactgaagtcagtcaaagaatt
attggggataacgattatggagcgctcgtcttttgaaaagaaccccatc
gacttccttgaggcgaaaggttacaaggaagtaaaaaaggatctcataa
ttaaactaccaaagtatagtctgtttgagttagaaaatggccgaaaacg
gatgttggctagcgccggagagcttcaaaaggggaacgaactcgcacta
ccgtctaaatacgtgaatttcctgtatttagcgtcccattacgagaagt
tgaaaggttcacctgaagataacgaacagaagcaactttttgttgagca
gcacaaacattatctcgacgaaatcatagagcaaatttcggaattcagt
aagagagtcatcctagctgatgccaatctggacaaagtattaagcgcat
acaacaagcacagggataaacccatacgtgagcaggcggaaaatattat
ccatttgtttactcttaccaacctcggcgctccagccgcattcaagtat
tttgacacaacgatagatcgcaaacgatacacttctaccaaggaggtgc
tagacgcgacactgattcaccaatccatcacgggattatatgaaactcg
gatagatttgtcacagcttgggggtgactctggtggttctggaggatct
ggtggttctactaatctgtcagatattattgaaaaggagaccggGaagc
aactggttatccaggaatccatcctcatgctcccagaggaggtggaaga
agtcattgggaacaagccggaaagcgatatactcgtgcacaccgcctac
gacgagagcaccgacgagaatgtcatgcttctgactagcgacgcccctg
aatacaagccttgggctctggtcatacaggatagcaacggtgagaacaa
gattaagatgctcTCTGGTGGTTCTGgaggatctggtggttctactaat
ctgtcagatattattgaaaaggagaccggGaagcaactggttatccagg
aatccatcctcatgctcccagaggaggtggaagaagtcattgggaacaa
gccggaaagcgatatactcgtgcacaccgcctacgacgagagcaccgac
gagaatgtcatgcttctgactagcgacgcccctgaatacaagccttggg
ctctggtcatacaggatagcaacggtgagaacaagattaagatgctcga
ggatctggtggttctGCTAGCGACTATAAGGACCACGACGGAGACTACA
AGGATCATGATATTGATTACAAAGACGATGACGATAAGTAAgtttacca
cgatcgttatgctgagtatgttaagctctttatgactgtttttgtagtg
gtatagagtactgcagaatacagtaagctgctctattgtagcatttctt
gatgttgcttagtcacttatttcataaacaacttaatgttctgaataat
ttcttactaaacattttgttattgggcaagtgattgaaaatagtaaatg
ctttgtgtgattgaaaacccgctgatcagcctcgactgtgccttctagt
tgccagccatctgttgtttgcccctcccccgtgccttccttgaccctgg
aaggtgccactcccactgtcctttcctaataaaatgaggaaattgcatc
gcattgtctgagtaggtgtcattctattctggggggtggggtggggcag
gacagcaagggggaggattgggaagacaatagcaggcatgctggggatg
cggtgggctctatgg
Nucleotide sequence of BE4-mitoATP5-Flag-NES-
bGHpoly(A):
(SEQ ID NO: 13)
GCCACCATGTTGGGGTTTGTGGGTCGGGTGGCCGCTGCTCCGGCCTCCG
GGGCCTTGCGGAGACTCACCCCTTCAGCGTCGCTGCCCCCAGCTCAGCT
CTTACTGCGGGCCGCTCCGACGGCGGTCCATCCTGTCAGGGACTATGCG
GCGCAAGATTACAAGGACGACGATGACAAGGTGGATGAAATGACCAAAA
AGTTCGGCACGCTCACCATTCACGACACCGAAAAGGCCGCCGAATTCag
ctcagagactggcccagtggctgtggaccccacattgagacggcggatc
gagccccatgagtttgaggtattcttcgatccgagagagctccgcaagg
agacctgcctgctttacgaaattaattgggggggccggcactccatttg
gcgacatacatcacagaacactaacaagcacgtcgaagtcaacttcatc
gagaagttcacgacagaaagatatttctgtccgaacacaaggtgcagca
ttacctggtttctcagctggagcccatgcggcgaatgtagtagggccat
cactgaattcctgtcaaggtatccccacgtcactctgtttatttacatc
gcaaggctgtaccaccacgctgacccccgcaatcgacaaggcctgcggg
atttgatctcttcaggtgtgactatccaaattatgactgagcaggagtc
aggatactgctggagaaactttgtgaattatagcccgagtaatgaagcc
cactggcctaggtatccccatctgtgggtacgactgtacgttcttgaac
tgtactgcatcatactgggcctgcctccttgtctcaacattctgagaag
gaagcagccacagctgacattctttaccatcgctcttcagtcttgtcat
taccagcgactgcccccacacattctctgggccaccgggttgaaatctg
gtggttcttctggtggttctagcggcagcgagactcccgggacctcaga
gtccgccacacccgaaagttctggtggttcttctggtggttctgataaa
aagtattctattggtttagccatcggcactaattccgttggatgggctg
tcataaccgatgaatacaaagtaccttcaaagaaatttaaggtgttggg
gaacacagaccgtcattcgattaaaaagaatcttatcggtgccctccta
ttcgatagtggcgaaacggcagaggcgactcgcctgaaacgaaccgctc
ggagaaggtatacacgtcgcaagaaccgaatatgttacttacaagaaat
ttttagcaatgagatggccaaagttgacgattctttctttcaccgtttg
gaagagtccttccttgtcgaagaggacaagaaacatgaacggcacccca
tctttggaaacatagtagatgaggtggcatatcatgaaaagtacccaac
gatttatcacctcagaaaaaagctagttgactcaactgataaagcggac
ctgaggttaatctacttggctcttgcccatatgataaagttccgtgggc
actttctcattgagggtgatctaaatccggacaactcggatgtcgacaa
actgttcatccagttagtacaaacctataatcagttgtttgaagagaac
cctataaatgcaagtggcgtggatgcgaaggctattcttagcgcccgcc
tctctaaatcccgacggctagaaaacctgatcgcacaattacccggaga
gaagaaaaatgggttgttcggtaaccttatagcgctctcactaggcctg
acaccaaattttaagtcgaacttcgacttagctgaagatgccaaattgc
agcttagtaaggacacgtacgatgacgatctcgacaatctactggcaca
aattggagatcagtatgcggacttatttttggctgccaaaaaccttagc
gatgcaatcctcctatctgacatactgagagttaatactgagattacca
aggcgccgttatccgcttcaatgatcaaaaggtacgatgaacatcacca
agacttgacacttctcaaggccctagtccgtcagcaactgcctgagaaa
tataaggaaatattctttgatcagtcgaaaaacgggtacgcaggttata
ttgacggcggagcgagtcaagaggaattctacaagtttatcaaacccat
attagagaagatggatgggacggaagagttgcttgtaaaactcaatcgc
gaagatctactgcgaaagcagcggactttcgacaacggtagcattccac
atcaaatccacttaggcgaattgcatgctatacttagaaggcaggagga
tttttatccgttcctcaaagacaatcgtgaaaagattgagaaaatccta
acctttcgcataccttactatgtgggacccctggcccgagggaactctc
ggttcgcatggatgacaagaaagtccgaagaaacgattactccatggaa
ttttgaggaagttgtcgataaaggtgcgtcagctcaatcgttcatcgag
aggatgaccaactttgacaagaatttaccgaacgaaaaagtattgccta
agcacagtttactttacgagtatttcacagtgtacaatgaactcacgaa
agttaagtatgtcactgagggcatgcgtaaacccgcctttctaagcgga
gaacagaagaaagcaatagtagatctgttattcaagaccaaccgcaaag
tgacagttaagcaattgaaagaggactactttaagaaaattgaatgctt
cgattctgtcgagatctccggggtagaagatcgatttaatgcgtcactt
ggtacgtatcatgacctcctaaagataattaaagataaggacttcctgg
ataacgaagagaatgaagatatcttagaagatatagtgttgactcttac
cctctttgaagatcgggaaatgattgaggaaagactaaaaacatacgct
cacctgttcgacgataaggttatgaaacagttaaagaggcgtcgctata
cgggctggggacgattgtcgcggaaacttatcaacgggataagagacaa
gcaaagtggtaaaactattctcgattttctaaagagcgacggcttcgcc
aataggaactttatgcagctgatccatgatgactctttaaccttcaaag
aggatatacaaaaggcacaggtttccggacaaggggactcattgcacga
acatattgcgaatcttgctggttcgccagccatcaaaaagggcatactc
cagacagtcaaagtagtggatgagctagttaaggtcatgggacgtcaca
aaccggaaaacattgtaatcgagatggcacgcgaaaatcaaacgactca
gaaggggcaaaaaaacagtcgagagcggatgaagagaatagaagagggt
attaaagaactgggcagccagatcttaaaggagcatcctgtggaaaata
cccaattgcagaacgagaaactttacctctattacctacaaaatggaag
ggacatgtatgttgatcaggaactggacataaaccgtttatctgattac
gacgtcgatcacattgtaccccaatcctttttgaaggacgattcaatcg
acaataaagtgcttacacgctcggataagaaccgagggaaaagtgacaa
tgttccaagcgaggaagtcgtaaagaaaatgaagaactattggcggcag
ctcctaaatgcgaaactgataacgcaaagaaagttcgataacttaacta
aagctgagaggggtggcttgtctgaacttgacaaggccggatttattaa
acgtcagctcgtggaaacccgccaaatcacaaagcatgttgcacagata
ctagattcccgaatgaatacgaaatacgacgagaacgataagctgattc
gggaagtcaaagtaatcactttaaagtcaaaattggtgtcggacttcag
aaaggattttcaattctataaagttagggagataaataactaccaccat
gcgcacgacgcttatcttaatgccgtcgtagggaccgcactcattaaga
aatacccgaagctagaaagtgagtttgtgtatggtgattacaaagttta
tgacgtccgtaagatgatcgcgaaaagcgaacaggagataggcaaggct
acagccaaatacttcttttattctaacattatgaatttctttaagacgg
aaatcactctggcaaacggagagatacgcaaacgacctttaattgaaac
caatggggagacaggtgaaatcgtatgggataagggccgggacttcgcg
acggtgagaaaagttttgtccatgccccaagtcaacatagtaaagaaaa
ctgaggtgcagaccggagggttttcaaaggaatcgattcttccaaaaag
gaatagtgataagctcatcgctcgtaaaaaggactgggacccgaaaaag
tacggtggcttcgatagccctacagttgcctattctgtcctagtagtgg
caaaagttgagaagggaaaatccaagaaactgaagtcagtcaaagaatt
attggggataacgattatggagcgctcgtcttttgaaaagaaccccatc
gacttccttgaggcgaaaggttacaaggaagtaaaaaaggatctcataa
ttaaactaccaaagtatagtctgtttgagttagaaaatggccgaaaacg
gatgttggctagcgccggagagcttcaaaaggggaacgaactcgcacta
ccgtctaaatacgtgaatttcctgtatttagcgtcccattacgagaagt
tgaaaggttcacctgaagataacgaacagaagcaactttttgttgagca
gcacaaacattatctcgacgaaatcatagagcaaatttcggaattcagt
aagagagtcatcctagctgatgccaatctggacaaagtattaagcgcat
acaacaagcacagggataaacccatacgtgagcaggcggaaaatattat
ccatttgtttactcttaccaacctcggcgctccagccgcattcaagtat
tttgacacaacgatagatcgcaaacgatacacttctaccaaggaggtgc
tagacgcgacactgattcaccaatccatcacgggattatatgaaactcg
gatagatttgtcacagcttgggggtgactctggtggttctggaggatct
ggtggttctactaatctgtcagatattattgaaaaggagaccggGaagc
aactggttatccaggaatccatcctcatgctcccagaggaggtggaaga
agtcattgggaacaagccggaaagcgatatactcgtgcacaccgcctac
gacgagagcaccgacgagaatgtcatgcttctgactagcgacgcccctg
aatacaagccttgggctctggtcatacaggatagcaacggtgagaacaa
gattaagatgctctctggtggttctggaggatctggtggttctactaat
ctgtcagatattattgaaaaggagaccggtcatcatcaccatcaccatt
gagtttaaacccgctgatcagcctcgactgtgccttctagttgccagcc
atctgttgtttgcccctcccccgtgccttccttgaccctggaaggtgcc
actcccactgtcctttcctaataaaatgaggaaattgcatcgcattgtc
tgagtaggtgtcattctattctggggggtggggtggggcaggacagcaa
gggggaggattgggaagacaatagcaggcatgctggggatgcggtgggc
tctatgg
HIV-1 REVNES nucleotide sequence:
(SEQ ID NO: 14)
CTTCAGCTACCACCGATTGAGAGACTTACTCTT
HIV-1 REV NES amino acid sequence:
(SEQ ID NO: 15)
LQLPPIERLTL
ATP5F1P MTS nucleotide sequence:
(SEQ ID NO: 16)
ATGTTGGGGTTTGTGGGTCGGGTGGCCGCTGCTCCGGCCTCCGGGGCCT
TGCGGAGACTCACCCCTTCAGCGTCGCTGCCCCCAGCTCAGCTCTTACT
GCGGGCCGCTCCGACGGCGGTCCATCCTGTCAGGGACTATGCGGCGCAA
ATP5F1P MTS amino acid sequence:
(SEQ ID NO: 17)
MLGFVGRVAAAPASGALRRLTPSASLPPAQLLLRAAPTAVHPVRDYAAQ
NS2 protein of Minute Virus of
Mice NES nucleotide sequence:
GTGGATGAAATGACCAAAAAGTTCGGCACGCTCACCATTCACGACACCG
AAAAG
(SEQ ID NO: 18)
NS2 protein of Minute Virus of
Mice NES amino acid sequence:
(SEQ ID NO: 19)
VDEMTKKFGTLTIHDTEK

What is claimed is:

1. A composition comprising a delivery vehicle and a protein, said protein comprising a mitochondrial localization amino acid sequence covalently attached to a base editor fusion protein comprising a nucleotide base-modifying enzyme and an RNA-guided DNA endonuclease enzyme with modified endonuclease activity, wherein said protein is bound to said delivery vehicle.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. A composition comprising a delivery vehicle complexed with a nucleic acid, said nucleic acid encoding a mitochondrial localization amino acid sequence covalently attached to a base editor fusion protein comprising a nucleotide base-modifying enzyme and an RNA-guided DNA endonuclease enzyme with modified endonuclease activity.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. A protein comprising a mitochondrial localization amino acid sequence covalently attached to a base editor fusion protein, said base editor fusion protein comprising a nucleotide base-modifying enzyme and an RNA-guided DNA endonuclease enzyme with modified endonuclease activity.

26. (canceled)

27. (canceled)

28. (canceled)

29. (canceled)

30. (canceled)

31. (canceled)

32. (canceled)

33. (canceled)

34. (canceled)

35. A nucleic acid encoding said protein of claim 25.

36. (canceled)

37. A vector comprising the nucleic acid of claim 35.

38. (canceled)

39. (canceled)

40. A pharmaceutical composition comprising the composition claim 1 and a pharmaceutically acceptable excipient.

41. (canceled)

42. A method of altering expression of at least one mitochondrial nucleic acid sequence, the method comprising introducing into an eukaryotic cell the composition of claim 1 and a single-guide RNA (sgRNA).

43. (canceled)

44. (canceled)

45. (canceled)

46. A method of treating a mitochondrial disorder in a subject in need thereof, the method comprising administering to said subject an effective amount of the composition of claim 1 and single-guide RNA (sgRNA).

47. (canceled)

48. A kit, comprising the composition of claim 1 and instructions for use.

49. (canceled)

50. A pharmaceutical composition comprising the protein of claim 25 and a pharmaceutically acceptable excipient.

51. (canceled)

52. A pharmaceutical composition comprising the nucleic acid claim 35 and a pharmaceutically acceptable excipient.

53. (canceled)

54. A pharmaceutical composition comprising the vector of claim 37 and a pharmaceutically acceptable excipient.

55. (canceled)

56. A method of altering expression of at least one mitochondrial nucleic acid sequence, the method comprising introducing into an eukaryotic cell the protein of claim 25 and a single-guide RNA (sgRNA).

57. (canceled)

58. (canceled)

59. (canceled)

60. A method of altering expression of at least one mitochondrial nucleic acid sequence, the method comprising introducing into an eukaryotic cell the nucleic acid of claim 35 and a single-guide RNA (sgRNA).

61. (canceled)

62. (canceled)

63. (canceled)

64. A method of altering expression of at least one mitochondrial nucleic acid sequence, the method comprising introducing into an eukaryotic cell the vector of claim 37 and a single-guide RNA (sgRNA).

65. (canceled)

66. (canceled)

67. (canceled)

68. A method of treating a mitochondrial disorder in a subject in need thereof, the method comprising administering to said subject an effective amount of the protein of claim 25 and single-guide RNA (sgRNA).

69. (canceled)

70. A method of treating a mitochondrial disorder in a subject in need thereof, the method comprising administering to said subject an effective amount of the nucleic acid of claim 35 and single-guide RNA (sgRNA).

71. (canceled)

72. A method of treating a mitochondrial disorder in a subject in need thereof, the method comprising administering to said subject an effective amount of the vector of claim 37 and single-guide RNA (sgRNA).

73. (canceled)

74. A method of treating a mitochondrial disorder in a subject in need thereof, the method comprising administering to said subject an effective amount of the pharmaceutical composition of claim 40 and single-guide RNA (sgRNA).

75. (canceled)

76. A kit, comprising the protein of claim 25 and instructions for use.

77. (canceled)

78. A kit, comprising the nucleic acid of claim 35 and instructions for use.

79. (canceled)

80. A kit, comprising the vector of claim 37 and instructions for use.

81. (canceled)

82. A kit, comprising the pharmaceutical composition of claim 40 and instructions for use.

83. (canceled)


SEQUENCE LISTING (19 sequences)

SEQ ID NO: 1 - protein, 29 aa, Homo sapiens
MSVLTPLLLRGLTGSARRLPVPRAKIHSL

SEQ ID NO: 2 - DNA, 87 bp, Homo sapiens
atgtccgtcctgacgccgctgctgctgcggggcttgacaggctcggcccggcggctcccagtgccgcgcgccaagatccattcgttg

SEQ ID NO: 3 - DNA, 472 bp, Homo sapiens
ggctacggctgaccgttttttgtggtgtactccgtgccatcatgtccgtcctgacgccgctgctgctgcggggcttgacaggctcggcccggcggctcccagtgccgcgcgccaagatccattcgttgccgccggaggggaagcttgggatcatggaattggccgttgggcttacctcctgcttcgtgaccttcctcctgccagcgggctggatcctgtcacacctggagacctacaggaggccagagtgaaggggtccgttctgtccctcacactgtgacctgaccagccccaccggcccatcctggtcatgttactgcatttgtggccggcctcccctggatcatgtcattcaattccagtcacctcttctgcaatcatgacctcttgatgtctccatggtgacctccttgggggtcactgaccctgcttggtggggtcccccttgtaacaataaatctatttaaacttt

SEQ ID NO: 4 - DNA, 21206 bp, Homo sapiens
tcaagtgttttttttgttttgttttttgtttgtttgtttgtttgtttttgacactgagtcccactctgtcgcccaggctggagtgcagtggcgagatctcagctcactgcagctgccgcctcccgggttcaagcgattcttctgcctcagcctcccaagtagctgggactacaggtgcgtgccaccacgcctagctaatttttgtatttttagtaaagacagagtttcaccatattggccaggctggtctccaattcctgacctcatgatccacctgcctcagcctcccaaagtcctgggattataggcgtgagccactgggccctgccaatttttgtatttttagtagagatggggtttcaccatgttggccaggctgatctcgatttcccgacctcagatgatccgcccacctcggcctcccaaagtgttgggattacaggcgtgagccatggtgctgggcatatacgcagatttctgactgtgaagggaccagcacccctgacactgcatcattcaggggtcaactgtacattcatatcccccttactacctactagctctatgtgtgaccttggtcaagtcactgtctttgcttacagcaggaataaagcatttgacttagtagctagcaacatggaggatctgctcaatgatgccgactccctttgccctttatggacgtaatcaactcatgattacctgcgacacatgcccactttatccctttgtttaacagaacacttttgttctgaaccaagctcctcctttcctttccagtagttaatgtgttctttgaggatgcaatctaatcgtaattttgcccgaagtggggaaaaaaattaagctcacattgaaaccttaatatagctctataatcattttctcttttagcctagatcacttcattttggcaatttaagtactacttttggtcctattaacaataccactcttgcaaaggacaggttacaaccaattctcagattcctgcctggataagatttgtgaccccaaattttaaaaaagcagaaaaaaaccccacaaaccacaggcaggaagaaagaaaaacaagagaatgtagctaaggtaattccattaatgatcagtttatgtcaaagcatgtggaaataatgccttataggaggatatttagcaacaacgatttttttaaactcattttctcctgtaggtacagaattgcaagttttttctgacttcaaatgtgtatagctgactgcgtgtgtgtgtgtgtgtgtgtgtgtgtgtgtgtgtgtgtgtgtgtgtcagggtctccttctcttgcccagactggagtaaggtgacataatcatagctcattgcagccatgaactcttgggctcaagtgatcctcctgccttcagcctcctggcgtagctaggactgcaggcacatgctaccacacccagctactgttttttggtagagacagagtcttgctatgttgcctaagctggtctcaaactcctggcctcaagcaatcttcccacctcagcctctcaaagagctgggattacaagcatgaaccactgcacctggccccatacctgactacatttctcagcttcctttgtggctaggctgtgctaggtagccttgtacctaaattttgaccagtgagcagcaagtggaatttgttggatgaatctaccacaaaggatccttacaatggagatagtggggccaggcatggtggctcatgcctgtaatcccagcactttgggaggctgaggcaggcagatcacttcagtctaggagttcgagaccagcctggccaacatggtgaaaccccatctccactaaaaatacaaaaattagccaggcatggtggcacgcacctgtaatcccagctactcaggaggctaaggcaggagaatcacttgaactcaggaggtggaggttgcagtgagccgagatcgcaccactgcactccagcccagcaacaaagcaagactcttgattcggaaagaaaaaataaaaaaaggttgggggagacagtgggagcccagacttttgtccttccccttgcctagaagggagatgaggttgctggtgctgtggaagctattatggaccatgaggcagctttgaagacagaaagcctgcatccttgatgactcaatggaatttcatcccaacccaggactgactgccccagatctttgttacatgagggaatacactcttctgtgtttaagaaactgtggttggatctgttactggaatatgaatgcagttaataactggctcaaaatgacttcatctcaacccacattggttgcttcttccaaacaatatataggtctgtactgtggtgggtctcaggatggctgtgatgtagccttaggaagtttatctatgggaaatccatattcatggtgtcctgatgttgcagaggacatcctgagctggctggagtaacttgggacacaggtcaatcgactgtaatctaacttctgaggccattcagtaccctctacagtggccacctaaaaaaaaggcagccaggtgtggtggctcaagcctatatagatcccagaactttggaaggctgaggtgggaggatcacttgagccaaggagtttgaggccagcctgggcaacatgaagaaactctgtttctacaaaaaataaaaaaaattagccaggcatggtggtatgcacctgtagttccagcttcttgggaggctgaggtggaagaatgacatgagcccagcaagtcgctgcaatcagccgtgatcacgccgctgcactccagcctgggcgacaaaaagaaaaagaaaacggagcctgttcactgggtgtggtagacaaggtaaacttttctttacctcccatatcccacaaccttggatgtgctcacagtcatggtagtgttttgtaatgatgtagctgatgacaggtgtgatgttggagattcttctacctgactgctgctatcagtcctaccagcccccaacgtttggtgcttgttctaaagggcatgtcctaggagtcgctttaaactctcaaagtatcactctctatacaaacaagaagtgcaagtaagtagcctgagctcagcctcccaataggaatatttcattatcactagatcaagtctttccattacaatgactgatctgtctctgaatcctgtggattcatccttcaaaatgcccttttcttccagttctcatagctagtgccctaaaagtgacctgcagtacctcctgctgagacgaatgtaccagcttcctaactagcctgcactcccttcatccccccaagtcagtgccagaccaccttgcctgaaaaaccactttcagtgtgtctcacctcagcagaaatgtttctcagcttccaattaacaatcacatcaaacccctgctcttgtctgcgttttaagggtatctataggccgggcgccgtggctcctacctgtaatcccagcactttggaaggccgaggcgggcagatcacttgaggtcaggcgttcgagaccatcctgaccaacatagtgaaaccccgtctctaccaaaaatacaaaaaaaaaaaaaatagtggggcgtggaggtgcacgcctgtaattccagctactcgggaggctgaggcaggagaatcgcttgaacccgggaggcagaggttccagtgagccgacatcgcgacacagtactccagcctgagcgacagagcgaggctgtgtctcaaaaataaataaataataaattaaaaaaataagagtatctataacctggtcccagcctgaatttcctttttcaccccaacacgtagccctagttacattcttctgacgtctgtaaacaagcccagcccttcctgttgtgaagccaagttcaggtggttcctcttcgcctgactgttttcccattccacttaccggaagcctagtcatccttcggagggctgtacgggggttgcaagaagcaacggaaacggttcagcacctgctaccttccatcatattcttttcaataaaggggcaactcccgccaatggcagtgtagatttcctaacctctacacatggaagattcacaccattcaggattgttgtttaactgttgagagagcacttgatacttaacagcttactaggctacaagacagcgcaggaaagaatcctctgttgtccttttatgttatcctgaacagttggttcacagagttactgtaaacacacaaaacatgactgccagggcttagtagtgaggaaggtgggaactagtcctgactcagttaactgtgcccaggagaagctgcttaacctcaaaggatttcactattactagaatcaataataccaaccctaggggtaaaaataaagataaatgtgtgcaaatcctgcctgcagtctcgggcacgtcgtgggtgtccaagaactgttcttaggcagccggtggggacaaagtctgtgtgcctcctgtcctggaataggtcccaaggtcggcttacttgcaaagcaagggtacggcgcaagagtactgaatacgggttggaagggcgctggctctaccctcagctcataggccggctgggcggcgctgaccagcagctaggccccgtcttccctaggaacggccacgggggccctgggagggtatgaatgtctttttgcagtgaggcctctggaccccgcggccccccggcagcgcaaccaaaactcaggggcaggcgccgcagccgcctagtgcagccagatccccgccggcaccctcaggggcggagccggaggcagggccttcgggccgtaccaactccacgggggcaggggccgcctcccttcggccgcgcgccactcaagtacggcagacaggcagcgaggttgccgaggccgaggctagcctgcagcctcctttctcccgtgccctgggcgcggggtgtacggcaagcgcgggcgggcgggacaggcacgcagggcacccccggggttgggcgcggcgggcgcggggcggggcccgcggggggggggggcggggcggcggtgcccttgcggcgcagctggggtcgcggccctgctccccgcgctttcttaaggcccgcgggcggcgcaggagcggcactcgtggctgtggtggcttcggcagcggcttcagcagatcggcggcatcagcggtagcaccagcactagcagcatgttgagccgggcagtgtgcgggtgagaagaaaggggacccggtcacggccccaagggcgaaggggctcgcggcgggcagggcctccgcggcaatggcgacagtggccgcaccgggcctggcgggaccggggcacctgcaggcggttctcccgggagtgcccggcgcggcggctggagcggggatccgcagggaggggacgcggggactcgggggacgccgcgcgctgccgttcctcggcagcccagcctgcgtagacggtcccgcggcgctgactgaccgggctgtgctttctcgtcttcagcaccagcaggcagctggctccggttttggggtatctgggctccaggcagaagcacagcctccccgacctgccctacgactacggcgccctggaacctcacatcaacgcgcagatcatgcagctgcaccacagcaagcaccacgcggcctacgtgaacaacctgaacgtcaccgaggagaagtaccaggaggcgttggccaagggtaggttccaggctgagcggcgggaggcagtccccggcagaggcgaccccagggagccaggccccatacggacgggcctctccgtggaggagaactcgcttcgtatttgtaccggttccgagttttccaggcacgatagtctctcttttaaacacatggtctacctcattgtagaaggagtgcctcgatgggtttgaacacacttctgtcatctcagggaacttggggtcctgcgaaggagcttgccttactgttgtgagccacattccgttacacatattgccagcactggtgaattgtagggcctgaaaagaaagctctactgtgtcactcgttttttttgcaaattgaaattgttcttgttgtataatgtgctttggggaaatgtttggtctctcaggtaggtgtgccagccgtttgcaggagggctgagagcgcctgtccactggtggccagacatcatcgggtccgcaggtgtctctgagtgtcagggtcacctcctgatagaagtgggagtggtgtcttactgccaggtcacactgaaggtgggagacaggaggacactactccgtgctaggaaccatggtccttgtcatcttcctgagagcaaacggggtcgggactccagcctaggacttggagactccctgcatccccagctaccctgcaggtgaaacttcactgagcccacttgacttcaggtgggagaggaggaggcacaccgttgtggtgtgcgttaccaaaaaagcagtaacctaactgtggaacagttcaggatctttagacttgatacttttcttgatttcccttaggggtcaaagttcacaaggaaaatagccctttagtgggaaactgaaacaggcttgtttttatttactatttactcatatttgtaatgcaattaaaatatatgttgttaactttttttttttttctcaagagctcggttgataaaaccaggggtatgtggactttttgagtctgtgccttttgggggtgtgtatgggaatgttatgttttaacgttttttacaaaatagtattgtctttttaatttattcgttagtggtttgcacaaggaagataatcgatagtcatgttttttagactctctgtattgcttggtaagctacgtagtaaaaaatgtttacttttccttaaatgttttgaatttcggggttatgaaatttgttgagtaatttttagacagtcacatcttgttgactggaggcatctagtggaaaaatgcagtatttcagcctgattgtgtttgaagtaaatgattaaaagaggaggaagttaccacattctggaagatttacttgagacagacgaaccttgaattacgggaaaaggccccgtgatttaggaaataacaaatttgggaaacatgtaatggggagagactggggaataccccagttgtgaaagtacttcctgtaaggcaacatctgacaccaggaacctttctcttcagtattttaaaaacaacttaatttcagtcctttacttgtggaatcagagccttacttatgtaatacaacccactggaaaaaagctttttattgtattgtactatattgtttataagtgattgagtacctgcagagctttcttttacttaaacatattttaaaaattattaaaaagattttcatgtttgaaaacttggggaaaaaagataaaacaatgtcataatctcatcacatttaactgactaatatgaatatttttcaacaaaaagcatttgacttttattattatttgaggcaaaaactaaactgaaaccaaataaaatgttgaatttcagaaaagtgttcttaaaagcagatattaagccctcaatttttattaattttaaaatgagtaagcaaactgtaagatgttaagattattaaattatctctcattaagcatgtatctttcccattttaaaaattcagttactattgatttattaaaaaacaatagaaaagttacatctatttttagagtgactctcaagaaaatttaataagggaaacacagatctataaaaaactgtttgtaaacctaacaaaggaaaaaaaaagttggtgtctttaaaaaaccctttgagattagctgtgtattcagatatcagttggttctctcctttctgaatcgctaaacaactggagcttgtttcagaaagcagcattgtctacgtatgttccttttaaaaattttttttttaattctgtctaaaaaaaacagacggggtcacactatgttgcccaggctgctcttgaattcctgggctcccctatccacgcacctcggccttccagagtgctgggattacaggtgtgagccaccgtgcctagtcagacatacttttttttttttttttttttttgacggaatctcagtctgtcccgcaggttggagtgcagtgatgtgatctcggctcactacaacctccgtctcctgggttcaagcaattctcctgcctcagcctcctgagtagctgggattacaggcgtacgccaccacgcctggctactttttgtgggttttttagtagagacagggtttttccatgtcgtccaggctggtctggaactcctgacctcaagtgatccgcccatctcagcctcccaaagtgctgggattacaggcgtgagccacctcacctggccttgtatgtatgtttttttctttctctttctttttttttttctgtgacggagtattgccctgtcgcccaggctggagtgcagtggcgtgttcttggttcactgcaacctctgcctcctgggttcaagccattctcctgcctcatgctcctgagtagctgggattataggcacgcgccaccatacctggctaatttctgtatttttagtgaagacagggtttcaccatgttggccaggccggtcttgaaatcctgacctgaagagagctgcccgcctcggcttcccaaagtgctgagattacaggcgtgagccaccgcacccggcccagacatatgttcttaacgtacactttcagaacagcagttgagaactttttccaaaatgtaagctccttgagagcaaagctctcagtgggtctctgtgtcctttactttgcttctcttggtatcttgtggataatatgcacaaaatatttattaaacaagaggatttccatttttttctaatattaatagttcatttagtctgtagcttcttgaaggcatagaaaagactattttattttgcctacttatttggcaaggtacccatgcagtatgccaggagttacagggcctattgagaaggagtaaagggtctgggaggggtggcagggagttgctaattcatgtatggtggctagagaaggcttcactgatggggttgatgtttcagtggagacttgaaggaggaacaggaacgcagcatgagactgtcttggggaagagtgttcagcagaggcagcagtgctcagctagccttgaggggaacatccaagttggtcatgtgagtgcaaaaaggccagtgaggctgcagctgcgtgagcacgtgctcaatgacggggtcaagtgcagcgcctcttggtagtaagagtagctgagtgtaaacacctagggttcgctccagtgagaaggggagcctttggagggggcaaggcccacccccaccccccaaaactgaggggatagtggcaccagtgcagtgagaaatactggtcctctgtcccaatgaggtggcagtagagttagggagaagggattaggttctgcttgtgttttatttttttcttttatttttctctgtgtgcttttatatagattggatgtattttaaaggtagagctggctttgctaatggatatgctggtggattggatgtggggtgaagggagaggagtcaaaaatgacatcaggttcccaaggatgtcctgagggacagagctggcatttactgagacgtggaggacaataggaggagtaagtttggggtagaacatcaggagttcaattttgagtatgttgagtttcttgttaatgtgacttggagatatttcccaatgaatcgtcgtctgtcttgaaagtgagagttgaaggttttctgtacagtctgtatgttgagccatacattttagcattctattattttgattgtcctctttggtgtttcatttcctaaaatctcttctatctaagagatccctgagcctagcagccaggagtgaattttctagcttttccacctctgctaggtccttcatcagttgttcagcatgtctgggacaaataattttggggcgggggggtgctgaattttaggacaaacggatgttatagataagctggtcccattatctaatagcttacaaagaaaaaaatataatgtatacagtggttgaaaaagtaggagttacaaaaaaatgtgtttgcattttaacttttcaggagatgttacagcccagatagctcttcagcctgcactgaagttcaatggtggtggtcatatcaatcatagcattttctggacaaacctcagccctaacggtggtggagaacccaaaggttggtatatattggtgcacccttatctacattttgtgcacagtaggaatcgattgttgatttactgaagttaccttatacttgttttttgttttttttgttgttgtttttttgagacaagagttttgctcttgttgcccaggctggagtacagtggcgcagtcttggctcactgcaacctccgcttcctgggttcaagtgattctcctgcctcagcctcccaagtagctgggattacaggcgcccgccaccacgcctggctaatttttgcatttttagtagagatgaggtttcattcaccatgttggccaggctggtcttgaactcctgacctcatgatctgcccaccttggcctcccaaagtgctgggattacaggcatgagccaccgtgcctggcctaagttaccttattcttaccagaggtatcatagtggcatatttttcataacaacgagtatgataaaagttgtaactttcaaaaggacacaagtaggaatggagaatgctttcatggggttatgtcataagattttatgttcacgccaacatagaatatcacatcaactccaccttctggtttctggatggtacttcatattagggaaagactgagagacttcattttatgaactttttttttttgagacggagtcttacagtcgcccaggctggagcgcagtggtgcaatgtcggctcattgcaacctccgcctttcaggttcatgcaattcttgtgtctcagccacccaagtagctgggattacaggcatgtggcatcactcccggctaatttttatatttttggtagagacagggtttcaccatgttggcggggctggtctcgaactcctggcctcaagtgatccacccaccttggcctcccaaagtgctgtggtatggatgtgagccaccgcgccagtctagaaaagatgaactttctgtctactagtttctagggcctggattagatagttaaccgtctgtaccttttttcatctatgtgatacagggataacaatggtgttgttttgagaattaaattatagtgtcatttgctatttttcactctttcctaactgataaaaagtcagttgttccaggaaattaggttttctgggtatttttaaaaagtgttcagggaatgaggagagtacagggctagatggtcttgttcgctatcactgtttccttgacttcctgtgtagcttgtggaggcccagatgactttaattatcttcttcacccagggagggcatgaggcgtaaatgtagttcttgtctcttgtttctgggctccggatatgcaccctaagtttctgtggttttattgttcatttttaatcagtgttatggaaggcagcctatcttatgcacttgaaataagcaatttcattttaaggtaatgatgaatgaggagagtaagcttgggaaactgggcaactttttctttatttttgtttttatttattattattttggagatggagtttagctctttcgcccaggctacagtgaagtggcttgatcttggctcactgcagcctctgcccatcccgggttcaagtgattctctgcctcagcctcccgagtagctgggattataggtgcctgccaccatgcccggctaatttttttgtatttttagtagagaggagtttctccatgttggtcaggctggttttgaactcctgacctcaggtgatccacctgtctcggcctcccaaagtactaagattacaggtgtgagccaccgtgcctggccaacttattttttttctgagttcagtcttctagactatcggttaatacttttgaagttttgtcagttaccagaatatcagatatattcatatgcaaccagtggttttggtatgctgccatttttgtttaatctgtaccacattccatcatttgtgcttataaagatgattacttttaaacccgtagaataagagaagtgagattttgacccagttgatttagttgatgtgactaatctagaattatattctgtattactacagaataaaaggattacagctcttaaagtatacccttagttcagtcgtagagaaaactgcccatcaagacaggaagagctgggctgccttggagaagaacaatgactttattcccaaatggaggcgtagaagaaggagggtagaagtcaaaggaaaattttctattcagggaaaagaaaaagctaacagaaagtagtatttctttcctaacaaagatagtacagtgagtggggaaatccctctaacacgattcattaaaaaatgtgaactacgcagctggaggccattatcctaagcgaattaacccaggaacagaaaaccaaataccacatgtctcacttgcaagtggaagctaaacatggagtacttatggatataaaaatggcagcaacagacactgaggacagagcagggagggagaaagaaggggacggggggtgaaaactgttgtatactaagctcgctacctgggtgatggcatcaattgcaccccagacctcagcatcatatgatacccgtgtaacaaacctgtgcgtgtaccctctgaatttaaaataaaatttgaaccgggcacggtggctcacgcctgtattcccagcactttgggaggccaaggcgggtggatcacctgaggttgggagtttgagaccagcctgaccaacatggagaaaccctgtctctactaaaaacacaaaattagccgggcgtgatggcacgtgcctgtaatcgcagctgctcgggaggctaaggcaggagaattgcttgaacctgggaggcagaggttgcagtgacccgagatcatggcattgcactccagcctgggcaacaagagtgaaactccgtctcaaaaggtaaaaaatgaaaaaaaaaaaaaaaaaaaaaaaaagttgaagttataaaaaacaaaaacgtgaattaaaattgtgaaacttagatccaggtgtcgcattctgatgttgtctaatttcttgggccctatgacaaaaatattttaatacatgtaatataacattttactgtaattattgaaatctgttcatttgtgggtggttttggattttttttttaataggggagttgctggaagccatcaaacgtgactttggttcctttgacaagtttaaggagaagctgacggctgcatctgttggtgtccaaggctcaggttggggttggcttggtttcaataaggaacggggacacttacaaattgctgcttgtccaaatcaggatccactgcaaggaacaacaggttagatttaaaaattgtgatttcatttgggagagatgctctactgtaaagcattcaactagaaataaggaaaactaacagtgttttaagaacatgtaataatttgcaaatcttaacagataacacccagagtcttgtgtaagtaaaaatgttttagaagtcctgatatttcataaaattaagtacacgtaattttataggtacattaacaaacatgttttatttttatttattttttttgagacagatctcactgttgcccaggctggagtgcagtggtgcgatctcggttcactgcaacctccgcctcctgggttcaagtgattctcctgcctcagcctcccaaatagctgggattacaggcacataccaccatgcccagctaatttttgtgtttttagtacagacagggtttcaccatgttggccaggctggtcttgaactcctgacctcaggtatctgcctgcctcggcctcctgaagtgctgggattacaggcatgagccaccatgcccagccaaatgttttacttattaaccctgcttcattctacagagaattaatatcagcttagataattatatgtactaaaataaaaaaaaaattagctatagaacaagagtgaggaaaagaaatacaagcagagactgatttcagagcttgtactgttacctcctctgctgagcatttgaggttaaactgggactagatctcaggtttccagaccttctgcctttctggggctctttggtagagtatatgaaaatattcggcttggaagtgccaggctgggctcaccaaatactttgctgttgagcctaattggatttaaaaaatgtcactgaagagtatgggaaatagagccatgagttgtctggcagagggttgggactttgaacttaaagtgataccttcacatcaacaacagtcattctctgccccttcttctacccctgacattggaatactcttgatctgtggctcacttcacaggatagacccagattttggcagaatctgaacagctcttttgggacagtgacatggcataacaatttgtaaaagccacaaaaaatttgtaagcaccgatctgtagagtcaggttatttgtagagctgcatgaccagaaaggctgatatttgtaagtgagtcccagctacactccttcctctccctgtggaaagcacattagaaggaaaatgagggaatgtcatggccttttgtttataaaaatcattaataaattttgctacttgaaattattaataataaattactagtgacctccctcacaactgatcaagacatactggttaagaattgtttttgtagagagagctataatttattttgagtgtttttgtgtgtgcgtgatggagtctcactctgttgcccaggctggagtgcagtggcgcaatcttggctcactacaacttccgcctcccaggttcaagcaattctcctgcgtcagcttcctgagtagctgggattacaggcgcctgccacctcgccaggctaattttttgtatttttagtagaggcggggtttcaccatgttggccaggctggtctcgaacgcctgacctcaagagatctgcctgcctcggcctcccaaagtgctgggattacaggcctgagccactgcgcccagaccaaaagtttttttaaatggatgttttacagatagaactaaattcttcatttacctcacttaatttctgaaaacactgtgtcctccaactgctctgtattcccaaagattaaaattagccttggggagcagagctggttcctcgtaaacccctgtgagtgcccagaccctttggaggaggttctcagtgggcttttgcctgctcccacagatgtaacccacccttcctgctgctcctaagaccaccatgaggaaggctgggaattggctcttgaattaatttgttggtatttttaagaatagctattaaagtactgttgttaattttcccaagtctattttattgttagtatttgtcacattttctttgtaaacagtgaattaaaaatattttataggaatattcattcttatctagatttcctacttatataaaaaaacaagtgaatgagattgttacaaagggtaattttgtgtgagtagaataataaaagttgaaattgagaagatgcaatgttttagactgaaactgatggttggtttgttttccccttctttctaacaggccttattccactgctggggattgatgtgtgggagcacgcttactaccttcagtataaaaatgtcaggcctgattatctaaaagctatttggaatgtaatcaactgggagaatgtaactgaaagatacatggcttgcaaaaagtaaaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaatctgattggacattttcttcagagagctaaattacaattgtcatttataaaaccatcaaaaatattccatccatatactttggggacttgtagggatgcctttctagtcctattctattgcagttatagaaaatctagtcttttgccccagttacttaaaaataaaatattaacactttcccaagggaaacactcggctttctatagaaaattgcactttttgtcgagtaatcctctgcagtgatacttctggtagatgtcacccagtggtttttgttaggtcaaatgttcctgtatagtttttgcaaatagagctgtatactgtttaaatgtagcaggtgaactgaactggggtttgctcacctgcacagtaaaggcaaacttcaacagcaaaactgcaaaaaggtggtttttgcagtaggagaaaggaggatgtttatttgcagggcgccaagcaaggagaattgggcagctcatgcttgagacccaatctccatgatgacctacaagctagagtatttaaaggcagtggtaaatttcaggaaagcagaagttaaaggcaaaattgtaaatcagtcgagatcgggtgccttcagggtggtatggctgtataccaaaattgtaaatcactacatgaagcttatatattggtttggcctgaaaggtgaagtggggtaggcagggggcgggcttacaggttatggtggattcaaagactccctgatttgtgattggttaaggaagcaaagctttgtctaaaaacttggggtccgcagaaaggaacattaaggtctggccaggcccctcaggaagaaactgagagcaaagaatggaggtcagagtttagtccctggtgttcccccttatctgacgtctgtgtgaatccatttggtgggggtctgggtttctgaaaagtagctcaggggcacgtgttaaggatgtctctaggtgactctaacttccctggctattgtttgaaactgttatgaccttcttgcttatcagcttgctggtttccttctcggggcgagctgggtgcctggagttttcggtgaaggaaactcaagattctcctttatttctgtgcttgtgggaatccccctggcacaccccaaagaggggtccctgctccgtctcacagggatctttttgtatatttggcttagcatcatacatttgccatgttgtttcatcatctgcctaatttactgtttttgaatatttcatttgtttctaattgttactacagataatgctggggtgagcaactctgtgtacataggtttatctcctattggaatattttctttatataggcgttttttttttttctttttttttggagacagagtcttgctctgttgcccaggctggagtgcagtggcgcgaccggagctcactgcaacctccacttcccgggttcaagtgattgtcccacctcagcctcctgaatagctgggattacaggtgcatgctaccatgcctggctactttttgtatttttagcagagacagggtttcaccatgttggccagggtggtctcgaactcctgacctcaagtgatccgtctggctcagcctcccaaagtgctgggattacaggtgtgagccactgcacctggcctatataggcttttttcttaaacctatttagtaatgttttcccaagtttattttttatttttaattttttccccaagtttatttttctatttttttttcatggaaaaatggggtaacttagcagtttcaatattgaagactgaagtttaaaaaaaatttaaattcaaggtacttttaaaattcagttagaaaagtaggctttaaaaattattagagacaagagtaccaaagcggtgtgtgtatgtgtgtgtgtgtatgcatgcttgtggattggaaaaactttggagactgattacttttcattatatatgtgtcacagtgaaacagcttttatgtgtcatgtaagattactgcttgcctctctaaggaaggtcgtgactgtttaaatagacgggcaaggtggaaccttttgaaagatgagcttttgaatataagttgtctgctagatcatggtttgtattgaactaacaaggtttgcagatctgctgacttatataaagctttttgattcctactaagctttaagatttaaaaaatgttcaatgttgaaatttctgtggggctctatttttgctttggctttctggtgagagagtgaggaagcattctttccttcactaagtttgtctttcttgtcttctggatagattgattttaagagactaagggaatttacaaactaaagattttagtcatctggtggaaaaggagactttaagattgtttagggctgggcggggtgactcacatctgtaatcccagcactttgggaggccaaggcaggcagaacacttgaaggagttcaagaccagcgtggccaacgtggtgaaaccctgtctctactaaaaatacaaaaattgtttagctctgtttttcataatagaaatagaaaaggtaaaattgcttttcttctgaaaagaacaagtattgttcatccaagaagggtttttgtgactgaatcagcagtgcctgccctagtcatagctgtgcttcaaaaacctcagcatgattagtgttggagcaaaacaaggaagcaaagcaaatactgtttttgaaattctatctgttgcttgaactattttgtaataattaaactttgatgttgagaaatcacaactttattgtacacttcattgcaacttgaaattcatggtcttaaagtgagatttgaatttctattgagcgcctttaaaaaagtaataccaaaccataaagttaaaatctatgtatattgagtcatatctaaaaccacgtataaacataaattgtatttcctgttttaattccaggggaagtactgtttgggaaagctattattaggtaaatgttttacaaattactgtttctcactttcagtcataccctaatgatcccagcaagataatgtcctgtcttctaagatgtgcatcaagcctggtacatactgaaaaccctataaggtcctggataatttttgtttgattattcattgaagaaacatttattttccaattgtgtgaagtttttgactgttaataaaagaatctgtcaaccatcaaagaggtctgcattatgcttgcatgtcaaaaactttaaaaatcctataatcttctgtcattttcactgagtttccatgggaaaggaatagtaaataatgggtagttgaaatattactcttaagaccaagacctgtatctccagtcatatctgtaataacatcatctgataacctaaaagcatagtattagggatatacgacaaaaccaaagtgtttttgctgttgtcacataccactcaatacttttacaccaggttgtccagtggacaccagctggatgtcctataattcaattctgacactgtctacctggaattggagtcagatcccacaggtggagggctcagcccacaagactgcccccactccagataccaatcgcaagtccctatacttctgactggctataaagtggggttcccgtgaccctttccttggttttgatttatttgctagagcagctcccagaacacagagaaacactctacttatgtttacccatttttaattttattactttattaaaactacatattggttacaatactaatttattgtaaaggatatttcatttatttatttttttatttagagatggagtctcactgtgtcacccaggctggagtgcggtggcacagtctcaactcactgcaacctccacctcctgagttcaagcgattctcatgcctcagcctcccaagtagctgggattacaagtgtgcaccaccatgcccagctaatttttgtatttttagtagagatggggtttcactctgttggccaggctggtctcaaactcctgacctcaagtcatccacccacctcagcctcccaaagtgctgagattacaggtgtgagccattgcctctggcttgtaaaggatatttcaaagaatacagatgaacagccagatggaagagatgcacagggcaaggcatgtgggaaggggtatggagctttcatgctctctccacgtcatcagctacccagaagctcatctggaccccgaccctttggggttttacggaggcttcattatgtcagcattaccattggcaattggcgattaactcaaccttcagcctctctcccctttctggaggttgggggttgaggctgggagtccaaaccctccaatcttgccttggtctttctggtaagcagccccccgtcctgaagctacctagaggctcccagccaccaatcatttcattggcaaaccaaagatacttcaaggggtttaggagccaagagaccaagcaaggattgagacaatatatatatttcttataaataacatgatcacaaatagtattttatatatttctcataatagctggttgtggttactcacacctatcatcccagctactaagaagctaaggcaggaggatcacaggcccaggagtttgcagctgtagtgagctatgattgtgctactgcactccagcctgggtgacagtgtgagaccctgtctcttaaagttcttgtaacatcttagaacattaaccaaccacagattattgccttaactctgaaaaataaagtaatcatacttttacaagatgtattttacataaagttctggtgatattttactctggtcattggaatataggagcttaccacctggtaccactcttcctaaagtggtatactatttggtatagtactggcatactatttggaaagggatgtgaagttggcatgacctgtctagggtgaagtcatgctgtctgctattcattgtgggctttacagtaatgagagtgcttctgttgtcaagatgcttctgggacacgttaaaaaaaatttaagatttctcagtttgtgaaaaggaaggagcacgaatgctttcaagttcctatgacatgctagtcatctttcagtgtgtcccattgttgcacccagtagtctcgatgcccagcgtcacggtgattccaagtttgcctctctgcccactataatttgtgctccatgaagtcccaggacatgaggttct

SEQ ID NO: 5 - protein, 222 aa, Homo sapiens
MLSRAVCGTSRQLAPVLGYLGSRQKHSLPDLPYDYGALEPHINAQIMQLHHSKHHAAYVNNLNVTEEKYQEALAKGDVTAQIALQPALKFNGGGHINHSIFWTNLSPNGGGEPKGELLEAIKRDFGSFDKFKEKLTAASVGVQGSGWGWLGFNKERGHLQIAACPNQDPLQGTTGLIPLLGIDVWEHAYYLQYKNVRPDYLKAIWNVINWENVTERYMACKK

SEQ ID NO: 6 - protein, 30 aa, Homo sapiens
MLSRAVCGTSRQLAPVLGYLGSRQKHSLPD

SEQ ID NO: 7 - DNA, 90 bp, Homo sapiens
atgttgagccgggcagtgtgcggcaccagcaggcagctggctccggttttggggtatctgggctccaggcagaagcacagcctccccgac

SEQ ID NO: 8 - DNA, 214 bp, Homo sapiens
accacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattga

SEQ ID NO: 9 - DNA, 5763 bp, Artificial Sequence
  Synthetic polynucleotide
gccgccaccatgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctggaggatctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctggaggatctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 10 - DNA, 5893 bp, Artificial Sequence
  Synthetic polynucleotide
gccaccatgtccgtcctgacgccgctgctgctgcggggcttgacaggctcggcccggcggctcccagtgccgcgcgccaagatccattcgttggatccgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctggaggatctggtggttctactaatctgtcagatattattgaaaaggagaccgggaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctggaggatctggtggttctactaatctgtcagatattattgaaaaggagaccgggaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctggaggatctggtggttctgctagcgactataaggaccacgacggagactacaaggatcatgatattgattacaaagacgatgacgataagtaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 11 - DNA, 5926 bp, Artificial Sequence
  Synthetic polynucleotide
gccaccatgtccgtcctgacgccgctgctgctgcggggcttgacaggctcggcccggcggctcccagtgccgcgcgccaagatccattcgttggatccgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctggaggatctggtggttctactaatctgtcagatattattgaaaaggagaccgggaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctggaggatctggtggttctactaatctgtcagatattattgaaaaggagaccgggaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctggaggatctggtggttctcttcagctaccaccgattgagagacttactcttgctagcgactataaggaccacgacggagactacaaggatcatgatattgattacaaagacgatgacgataagtaagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 12 - DNA, 6091 bp, Artificial Sequence
  Synthetic polynucleotide
atgttgagccgggcagtgtgcggcaccagcaggcagctggctccggttttggggtatctgggctccaggcagaagcacagcctccccgacgatccgagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctggaggatctggtggttctactaatctgtcagatattattgaaaaggagaccgggaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctggaggatctggtggttctactaatctgtcagatattattgaaaaggagaccgggaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctcgaggatctggtggttctgctagcgactataaggaccacgacggagactacaaggatcatgatattgattacaaagacgatgacgataagtaagtttaccacgatcgttatgctgagtatgttaagctctttatgactgtttttgtagtggtatagagtactgcagaatacagtaagctgctctattgtagcatttcttgatgttgcttagtcacttatttcataaacaacttaatgttctgaataatttcttactaaacattttgttattgggcaagtgattgaaaatagtaaatgctttgtgtgattgaaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 13 - DNA, 5740 bp, Artificial Sequence
  Synthetic polynucleotide
gccaccatgttggggtttgtgggtcgggtggccgctgctccggcctccggggccttgcggagactcaccccttcagcgtcgctgcccccagctcagctcttactgcgggccgctccgacggcggtccatcctgtcagggactatgcggcgcaagattacaaggacgacgatgacaaggtggatgaaatgaccaaaaagttcggcacgctcaccattcacgacaccgaaaaggccgccgaattcagctcagagactggcccagtggctgtggaccccacattgagacggcggatcgagccccatgagtttgaggtattcttcgatccgagagagctccgcaaggagacctgcctgctttacgaaattaattgggggggccggcactccatttggcgacatacatcacagaacactaacaagcacgtcgaagtcaacttcatcgagaagttcacgacagaaagatatttctgtccgaacacaaggtgcagcattacctggtttctcagctggagcccatgcggcgaatgtagtagggccatcactgaattcctgtcaaggtatccccacgtcactctgtttatttacatcgcaaggctgtaccaccacgctgacccccgcaatcgacaaggcctgcgggatttgatctcttcaggtgtgactatccaaattatgactgagcaggagtcaggatactgctggagaaactttgtgaattatagcccgagtaatgaagcccactggcctaggtatccccatctgtgggtacgactgtacgttcttgaactgtactgcatcatactgggcctgcctccttgtctcaacattctgagaaggaagcagccacagctgacattctttaccatcgctcttcagtcttgtcattaccagcgactgcccccacacattctctgggccaccgggttgaaatctggtggttcttctggtggttctagcggcagcgagactcccgggacctcagagtccgccacacccgaaagttctggtggttcttctggtggttctgataaaaagtattctattggtttagccatcggcactaattccgttggatgggctgtcataaccgatgaatacaaagtaccttcaaagaaatttaaggtgttggggaacacagaccgtcattcgattaaaaagaatcttatcggtgccctcctattcgatagtggcgaaacggcagaggcgactcgcctgaaacgaaccgctcggagaaggtatacacgtcgcaagaaccgaatatgttacttacaagaaatttttagcaatgagatggccaaagttgacgattctttctttcaccgtttggaagagtccttccttgtcgaagaggacaagaaacatgaacggcaccccatctttggaaacatagtagatgaggtggcatatcatgaaaagtacccaacgatttatcacctcagaaaaaagctagttgactcaactgataaagcggacctgaggttaatctacttggctcttgcccatatgataaagttccgtgggcactttctcattgagggtgatctaaatccggacaactcggatgtcgacaaactgttcatccagttagtacaaacctataatcagttgtttgaagagaaccctataaatgcaagtggcgtggatgcgaaggctattcttagcgcccgcctctctaaatcccgacggctagaaaacctgatcgcacaattacccggagagaagaaaaatgggttgttcggtaaccttatagcgctctcactaggcctgacaccaaattttaagtcgaacttcgacttagctgaagatgccaaattgcagcttagtaaggacacgtacgatgacgatctcgacaatctactggcacaaattggagatcagtatgcggacttatttttggctgccaaaaaccttagcgatgcaatcctcctatctgacatactgagagttaatactgagattaccaaggcgccgttatccgcttcaatgatcaaaaggtacgatgaacatcaccaagacttgacacttctcaaggccctagtccgtcagcaactgcctgagaaatataaggaaatattctttgatcagtcgaaaaacgggtacgcaggttatattgacggcggagcgagtcaagaggaattctacaagtttatcaaacccatattagagaagatggatgggacggaagagttgcttgtaaaactcaatcgcgaagatctactgcgaaagcagcggactttcgacaacggtagcattccacatcaaatccacttaggcgaattgcatgctatacttagaaggcaggaggatttttatccgttcctcaaagacaatcgtgaaaagattgagaaaatcctaacctttcgcataccttactatgtgggacccctggcccgagggaactctcggttcgcatggatgacaagaaagtccgaagaaacgattactccatggaattttgaggaagttgtcgataaaggtgcgtcagctcaatcgttcatcgagaggatgaccaactttgacaagaatttaccgaacgaaaaagtattgcctaagcacagtttactttacgagtatttcacagtgtacaatgaactcacgaaagttaagtatgtcactgagggcatgcgtaaacccgcctttctaagcggagaacagaagaaagcaatagtagatctgttattcaagaccaaccgcaaagtgacagttaagcaattgaaagaggactactttaagaaaattgaatgcttcgattctgtcgagatctccggggtagaagatcgatttaatgcgtcacttggtacgtatcatgacctcctaaagataattaaagataaggacttcctggataacgaagagaatgaagatatcttagaagatatagtgttgactcttaccctctttgaagatcgggaaatgattgaggaaagactaaaaacatacgctcacctgttcgacgataaggttatgaaacagttaaagaggcgtcgctatacgggctggggacgattgtcgcggaaacttatcaacgggataagagacaagcaaagtggtaaaactattctcgattttctaaagagcgacggcttcgccaataggaactttatgcagctgatccatgatgactctttaaccttcaaagaggatatacaaaaggcacaggtttccggacaaggggactcattgcacgaacatattgcgaatcttgctggttcgccagccatcaaaaagggcatactccagacagtcaaagtagtggatgagctagttaaggtcatgggacgtcacaaaccggaaaacattgtaatcgagatggcacgcgaaaatcaaacgactcagaaggggcaaaaaaacagtcgagagcggatgaagagaatagaagagggtattaaagaactgggcagccagatcttaaaggagcatcctgtggaaaatacccaattgcagaacgagaaactttacctctattacctacaaaatggaagggacatgtatgttgatcaggaactggacataaaccgtttatctgattacgacgtcgatcacattgtaccccaatcctttttgaaggacgattcaatcgacaataaagtgcttacacgctcggataagaaccgagggaaaagtgacaatgttccaagcgaggaagtcgtaaagaaaatgaagaactattggcggcagctcctaaatgcgaaactgataacgcaaagaaagttcgataacttaactaaagctgagaggggtggcttgtctgaacttgacaaggccggatttattaaacgtcagctcgtggaaacccgccaaatcacaaagcatgttgcacagatactagattcccgaatgaatacgaaatacgacgagaacgataagctgattcgggaagtcaaagtaatcactttaaagtcaaaattggtgtcggacttcagaaaggattttcaattctataaagttagggagataaataactaccaccatgcgcacgacgcttatcttaatgccgtcgtagggaccgcactcattaagaaatacccgaagctagaaagtgagtttgtgtatggtgattacaaagtttatgacgtccgtaagatgatcgcgaaaagcgaacaggagataggcaaggctacagccaaatacttcttttattctaacattatgaatttctttaagacggaaatcactctggcaaacggagagatacgcaaacgacctttaattgaaaccaatggggagacaggtgaaatcgtatgggataagggccgggacttcgcgacggtgagaaaagttttgtccatgccccaagtcaacatagtaaagaaaactgaggtgcagaccggagggttttcaaaggaatcgattcttccaaaaaggaatagtgataagctcatcgctcgtaaaaaggactgggacccgaaaaagtacggtggcttcgatagccctacagttgcctattctgtcctagtagtggcaaaagttgagaagggaaaatccaagaaactgaagtcagtcaaagaattattggggataacgattatggagcgctcgtcttttgaaaagaaccccatcgacttccttgaggcgaaaggttacaaggaagtaaaaaaggatctcataattaaactaccaaagtatagtctgtttgagttagaaaatggccgaaaacggatgttggctagcgccggagagcttcaaaaggggaacgaactcgcactaccgtctaaatacgtgaatttcctgtatttagcgtcccattacgagaagttgaaaggttcacctgaagataacgaacagaagcaactttttgttgagcagcacaaacattatctcgacgaaatcatagagcaaatttcggaattcagtaagagagtcatcctagctgatgccaatctggacaaagtattaagcgcatacaacaagcacagggataaacccatacgtgagcaggcggaaaatattatccatttgtttactcttaccaacctcggcgctccagccgcattcaagtattttgacacaacgatagatcgcaaacgatacacttctaccaaggaggtgctagacgcgacactgattcaccaatccatcacgggattatatgaaactcggatagatttgtcacagcttgggggtgactctggtggttctggaggatctggtggttctactaatctgtcagatattattgaaaaggagaccgggaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctggaggatctggtggttctactaatctgtcagatattattgaaaaggagaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 14 - DNA, 33 bp, Human immunodeficiency virus type 1
cttcagctaccaccgattgagagacttactctt

SEQ ID NO: 15 - protein, 11 aa, Human immunodeficiency virus type 1
LQLPPIERLTL

SEQ ID NO: 16 - DNA, 147 bp, Homo sapiens
atgttggggtttgtgggtcgggtggccgctgctccggcctccggggccttgcggagactcaccccttcagcgtcgctgcccccagctcagctcttactgcgggccgctccgacggcggtccatcctgtcagggactatgcggcgcaa

SEQ ID NO: 17 - protein, 49 aa, Homo sapiens
MLGFVGRVAAAPASGALRRLTPSASLPPAQLLLRAAPTAVHPVRDYAAQ

SEQ ID NO: 18 - DNA, 54 bp, Mice minute virus
gtggatgaaatgaccaaaaagttcggcacgctcaccattcacgacaccgaaaag

SEQ ID NO: 19 - protein, 18 aa, Mice minute virus
VDEMTKKFGTLTIHDTEK